(19) United States
(12) Patent Application Publication
(10) Pub. No.: US 2020/0123146 A1
(43) Pub. Date: Apr. 23, 2020

(54) HETEROCYCLIC INTEGRIN AGONISTS

(71) Applicant: GB006, Inc., San Diego, CA (US)

(72) Inventor: Antonio J. Barbosa, Ave Maria, FL (US)

(21) Appl. No.: 16/475,004

(22) PCT Filed: Dec. 28, 2017
(86) PCT No.: PCT/US2017/068825

§ 371 Date: Jun. 28, 2019

Related U.S. Application Data
(60) Provisional application No. 62/440,195, filed on Dec. 29, 2016.
(60) Provisional application No. 62/440,206, filed on Dec. 29, 2016.

(51) Int. Cl. C07D 417/14; C07D 263/46; C07D 413/06; A61P 35/00; C07D 277/36

(52) U.S. Cl. CPC C07D 417/14; C07D 263/46; C07D 277/36; A61P 35/00; C07D 413/06

(57) ABSTRACT

The present invention provides polycyclic oxothioxoimidazolidines, dioxoimidazolines, oxothioxooxazolidines, dioxooxazolidines, and related compounds, which are useful as integrin agonists. Methods for the treatment of integrin-mediated diseases such as cancer are also described.

### CROSS-REFERENCES TO RELATED APPLICATIONS

The present application claims priority to U.S. Provisional Pat. Appl. No. 62/440,195, filed Dec. 29, 2016, and U.S. Provisional Pat. Appl. No. 62/440,206, filed Dec. 29, 2016, which applications are incorporated herein by reference in their entirety.

### BACKGROUND OF THE INVENTION

Leukocyte (i.e., white blood cell) activation, migration and recruitment are essential for the immune response to injury and infection, as well as in various inflammatory and autoimmune disorders. The β2 integrins, a sub-family of α/β heterodimeric integrin receptors including highly expressed integrin CD11b/CD18, are leukocyte-specific receptors that modulate leukocyte functions including cell adhesion, migration, recruitment and activation. CD11b/CD18 recognizes the complement fragment iC3b, fibrinogen, and ICAM-1 as ligands, among various others. CD11b/CD18 has been implicated in many inflammatory and autoimmune diseases, such as ischemia-reperfusion injury (including acute renal failure and atherosclerosis), lupus, psoriasis, dermatitis, inflammatory bowel disease, Crohn's disease, rheumatoid arthritis, multiple sclerosis, lupus nephritis, focal segmental glomerulosclerosis, diabetic nephropathy, renal injury, tissue damage, glaucoma, uveitis, ophthalmic conditions, allograft rejection (such as nephropathy), transplantation, graft versus host disease, fibrosis, stroke, pain (including chronic pain), neointimal thickening in response to vascular injury, and the resolution of inflammatory processes.

Leukocytic β2 integrins also contribute to processes including tumor growth, tumor re-growth, tumor metastases, leukocyte infiltration into tumors, modulation of inflammation, modulation of anti-tumor responses, leukocyte polarization, modification of tumor microenvironment, production of reactive oxygen species, and modulation of a number of pro- and anti-inflammatory genes and proteins in inflammatory cells. Blocking of β2 integrins, including CD11b/CD18, and their ligands has been shown to decrease the severity of inflammatory response in vivo in certain experimental models. However, such blocking agents have had little success in treating inflammatory/autoimmune diseases in humans.

More recently, new anti-inflammatory compositions and methods have been developed using compounds that activate integrins and reduce recruitment of inflammatory immune cells into tissues by, for example, increasing integrin CD11b/CD18-dependent cell adhesion to immobilized ligands. Leukadherins are a group of such small molecule agonists targeting integrin CD11b/CD18 (Maiguel, et al. 2011. Sci. Signal. 4:1-14; Park, et al. 2007. J. Biomol. Screen. 12:406-417; Faridi, et al. 2009. Bioorg. Med. Chem. Lett. 19:6902-6906.). Leukadherins also reduce leukocyte activation and pro-inflammatory signaling pathways. Among them, leukadherin 1 (“LA1;” (Z)-4-(5-((3-benzyl-4-oxo-2-thioxothiazolidin-5-ylidene)methyl)furan-2-yl)benzoic acid) has demonstrated particular anti-inflammatory efficacy. LA1 has been shown to reduce recruitment of leukocytes during acute peritonitis in mice, reduce neointimal thickening upon vascular injury in rats, reduce hyperoxia-dependent lung injury, reduce liver fibrosis, reduce renal ischemia/reperfusion injury in mice, increase allograft survival, and enhance liver repair (Jagarapu, et al. 2015. Am J Respir Cell Mol Biol. 53: 793-801; Joshi, et al. 2016. Blood. 127: 2751-2762; Khan, et al. 2014. Front. Med. Vol. 1, Art. 45; Kopec, et al. 2016. J Hepatology, “Fibri(nogen) drives repair after acetaminophen-induced liver injury via leukocyte αMβ2 integrin-dependent upregulation of MMP12,” in press). LA1 also reduced tumor growth and re-growth. LA1 and uses thereof have been described in U.S. Pat. Nos. 9,023,876 and 9,328,105, as well as in International Pat. Appl. Nos. PCT/US2011/034753, PCT/US2013/037548, and PCT/US2016/037067, which patents and applications are incorporated herein by reference in their entirety. Improved integrin agonists are needed to further leverage the utility that this family of therapeutics has exhibited in the studies outlined above. Improved agonist activity, dissolution profiles, pharmacokinetic profiles, and/or stability profiles provided by the new compounds and formulations thereof will enhance efficacy and enable advantageous dosage forms. The present invention meets this and other needs.

### BRIEF SUMMARY OF THE INVENTION

In one embodiment, the invention provides a compound according to Formula I:

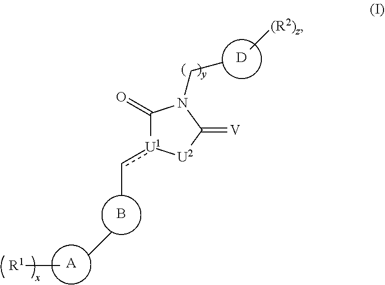

or a pharmaceutically acceptable salt thereof, wherein:
ring A is selected from phenyl and C₆ heteroaryl;
ring B is selected from phenylene and C₅₋₆ heteroarylene, each of which is optionally substituted with one or two C₁₋₄ alkyl;
ring D is selected from cyclohexyl and phenyl;
V is selected from O and S;
U¹ is selected from C, CH, and N;
U² is selected from O, C(R³)(R⁴), and NR^{a};
each R¹ is independently selected from halogen, (CH₂)_{w}COOR^{1a}, and C₅₋₆ heteroaryl, wherein C₅₋₆ heteroaryl is optionally substituted with (CH₂)_{w}COOR^{1a};
each R^{1a} is independently selected from H and C₁₋₆ alkyl;
each subscript w is 0, 1, or 2;
each R² is independently selected from halogen, C₁₋₆ alkyl, C₁₋₆ alkoxy, and R^{2a};
each R^{2a} is independently selected from C₂₋₆ alkyl and C₂₋₆ alkoxy;
each of R³ and R⁴ is independently selected from H and C₁₋₄ alkyl;
R^{a} is selected from H and C₁₋₄ alkyl;
subscript x is 0, 1, 2, 3, 4 or 5;
subscript y is 1, 2, or 0;
subscript z is 0, 1, 2, 3, 4, or 5; and
the dashed line represents a single bond or a double bond.

In another embodiment, the invention provides a compound according to Formula XI:

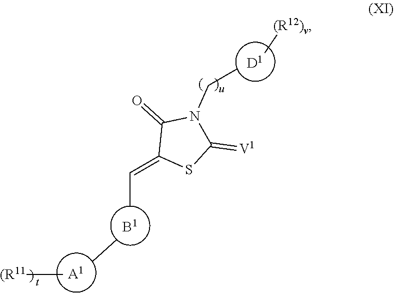

or a pharmaceutically acceptable salt thereof, wherein:
ring A¹ is C₆ heteroaryl;
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen and (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R^{12a};

each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4, or 5.

In another embodiment, the invention provides a compound according to Formula XIII:

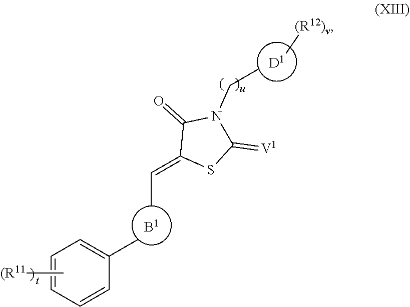

or a pharmaceutically acceptable salt thereof, wherein:
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen, (CH₂)ₛCOOR^{11a}, and C₅₋₆ heteroaryl, wherein C₅₋₆ heteroaryl is optionally substituted with (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R₁₂ₐ;

each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4, or 5.

In another embodiment, the invention provides pharmaceutically formulations containing the compounds described herein in combination with one or more pharmaceutically acceptable excipients.

In another embodiment, the invention provides methods for treating a β2 integrin-mediated conditions comprising administering to a patient in need thereof a compound as described herein or a pharmaceutically acceptable salt thereof In some embodiments, the integrin-mediated condition is selected from acute inflammation, chronic inflammation, chronic kidney disease, neointimal thickening associated with vascular injury, tissue injury, peritonitis, diabetic nephropathy, an autoimmune disease, cancer, glaucoma, graft versus host disease, macular degeneration, and uveitis.

### DETAILED DESCRIPTION OF THE INVENTION

### I. DEFINITIONS

“Alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, alkylamino, amido, acyl, nitro, cyano, and alkoxy.

“Alkoxy,” by itself or as part of another substituent, refers to a moiety having the formula —OR, wherein R is an alkyl group as defined herein. Examples of alkoxy groups include, but are not limited to, methoxy, ethoxy, and isopropyloxy.

“Cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. “Substituted cycloalkyl” groups can be substituted with one or more groups selected from halo, hydroxy, amino, alkylamino, amido, acyl, nitro, cyano, and alkoxy. The term “lower cycloalkyl” refers to a cycloalkyl radical having from three to seven carbons including, for example, cyclopropyl, cyclobutyl, cyclopentyl, cyclohexyl, and cycloheptyl.

“Aryl,” by itself or as part of another substituent, refers to an aromatic ring system having any suitable number of ring atoms and any suitable number of rings. Aryl groups can include any suitable number of ring atoms, such as 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. “Substituted aryl” groups can be substituted with one or more groups selected from halo, hydroxy, amino, alkylamino, amido, acyl, nitro, cyano, and alkoxy.

“Arylene,” by itself or as part of another substituent, refers to divalent aryl radical. Examples of arylene groups include, but are not limited to, o-phenylene, m-phenylene, p-phenylene, and naphthalene-2,3-diyl.

“Heteroaryl,” by itself or as part of another substituent, refers to a monocyclic or fused bicyclic or tricyclic aromatic ring assembly containing the number of carbon atoms indicated (e.g., 5 to 16 carbon ring atoms), where from 1 to 5 of the carbon ring atoms are replaced by a heteroatom such as N, O or S. Additional atoms can also be useful, including, but not limited to, B, Al, Si and P. The heteroatoms can be oxidized to form moieties such as, but not limited to, —S(O)— and —S(O)₂—. Heteroaryl groups can include any number of carbon ring atoms, such as 3 to 6, 4 to 6, 5 to 6, 3 to 8, 4 to 8, 5 to 8, 6 to 8, 3 to 9, 3 to 10, 3 to 11, or 3 to 12 carbon ring members. Any suitable number of carbon ring atoms can be replaced with heteroatoms, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 carbon ring members (i.e., C₅₋₈ heteroaryl) where 1 to 4 carbon ring atoms are replaced with heteroatoms; or from 5 to 8 carbon ring members where 1 to 3 carbon ring atoms are replaced with heteroatoms; or from 5 to 6 carbon ring members (i.e., C₅₋₆ heteroaryl) where 1 to 4 carbon ring atoms are replaced with heteroatoms; or from 5 to 6 ring members where 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

Some heteroaryl groups include those having from 5 to 10 carbon ring members where 1 to 3 carbon ring atoms are replaced with heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 carbon ring members where 1 to 3 carbon ring atoms are replaced with heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 carbon ring members where 1 to 3 carbon ring atoms are replaced with heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 carbon ring members where 1 to 2 carbon ring atoms are replaced with heteroatoms, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 carbon ring members wherein carbon ring atoms are replaced with only nitrogen atoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 carbon ring members wherein carbon ring atoms are replaced with only oxygen atoms, such as furan and benzofuran. Some other heteroaryl groups include from 5 to 10 carbon ring members wherein carbon ring atoms are replaced with only sulfur atoms, such as thiophene and benzothiophene. Still other heteroaryl groups include from 5 to 10 carbon ring members wherein carbon ring atoms are replaced with at least two types of heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroarylene,” by itself or as part of another substituent, refers to divalent heteroaryl radical. Examples of heteroarylene groups include, but are not limited to, pyridin-2,6-diyl, furan-2, 5 -diyl, and thiophen-2, 5 -diyl.

“Halo” and “halogen,” by themselves or as part of another substituent, refer to a fluorine, chlorine, bromine, or iodine atom

“Salt” refers to acid or base salts of the compounds of the invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

“Pharmaceutically acceptable” is art-recognized and, as used herein to refer to a composition, excipient, adjuvant, or other material and/or dosage form, refers to a substance which, within the scope of sound medical judgment, is suitable for use in contact with the tissues of human beings and animals without excessive toxicity, irritation, allergic response, or other problems or complications, commensurate with a reasonable benefit/risk ratio. Examples of pharmaceutically acceptable bases include, but are not limited to ammonia, L-arginine, calcium hydroxide, choline hydroxide, meglumine, lysine, magnesium hydroxide, potassium hydroxide, sodium hydroxide. It is understood that the pharmaceutically acceptable salts are non-toxic.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the present invention include, but are not limited to, binders, fillers, disintegrants, lubricants, glidants, coatings, sweeteners, flavors and colors.

“Integrin” refers to a non-covalently linked α/β-heterodimeric cell surface receptor that mediates cell adhesion, migration and signaling. Integrins are expressed in a wide range of organisms, including C. elegans, Drosophila sp., amphibians, reptiles, birds, and mammals, including humans. A number of α subunits, designated, for example, αV, α5 and the like, and a number of β subunits, designated, for example, β1, β2, β3, β5 and the like, have been identified, and various combinations of these subunits are represented in the integrin superfamily, including α5β1, αVβ3 and αVβ5. The superfamily of integrins can be subdivided into families, for example, as αV-containing integrins, including αVβ3 and αVβ5, or the ⊕1-containing integrins, including α5β1 and αVβ1.

“β2 integrin” refers to an integrin having a β2-subunit (also referred to as CD18). β2 integrins have distinct a-subunits selected from CD11a, CD11b, CD11c and CD11d. β2 integrins, including highly expressed integrin CD11b/CD18 (also known as Mac-1, CR3 and αMβ2), modulate cellular functions, including cell adhesion, migration, recruitment and activation.

“β2-mediated,” as used herein to refer to diseases and/or conditions in a patient, means that the disease or condition results (in whole or in part) from a chemical or physical process involving a β2 integrin. β2-mediated diseases and conditions include inflammatory, autoimmune, and neurodegenerative diseases. Examples of β2-mediated diseases and conditions include, but are not limited to, ischemia-reperfusion injury (including acute renal failure and atherosclerosis), lupus, inflammatory bowel disease, Crohn's disease, rheumatoid arthritis, multiple sclerosis, lupus nephritis, focal segmental glomerulosclerosis, renal injury, glaucoma, ophthalmic conditions, allograft rejection (such as nephropathy), transplantation, graft versus host disease, neurological disorders, Alzheimer's disease, Parkinson's disease, traumatic brain injury, dermatitis, tissue damage, stroke, neointimal thickening in response to vascular injury, anti-GBM nephritis, pain (including chronic pain), and cancers, including primary tumors and metastatic tumors, such as breast cancer, melanoma, prostate cancer, ovarian cancer, renal cancer, lung cancer, pancreatic cancer, glioblastoma, and others.

“Cancer” refers to an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. In general, a cancer will be associated with the presence of one or more tumors, i.e., abnormal cell masses. The term “tumor” is meant to include all types of cancerous growths or oncogenic processes, metastatic tissues or malignantly transformed cells, tissues, or organs, irrespective of histopathologic type or stage of invasiveness.

Examples of cancer include malignancies of various organ systems, such as lung cancers, breast cancers, thyroid cancers, lymphoid cancers, gastrointestinal cancers, and genito-urinary tract cancers. Cancer can also refer to adenocarcinomas, which include malignancies such as colon cancers, renal-cell carcinoma, prostate cancer and/or testicular tumors, non-small cell carcinoma of the lung, cancer of the small intestine, and cancer of the esophagus. Carcinomas are malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures. A “sarcoma” refers to a malignant tumor of mesenchymal derivation.

“Melanoma” refers to a tumor arising from a melanocyte. Melanomas occur most commonly in the skin and are frequently observed to metastasize widely.

The term “treating,” as used herein, unless otherwise indicated, means reversing, alleviating, inhibiting the progress of, or preventing the disorder or condition to which such term applies, or one or more symptoms of such disorder or condition. The term “treatment,” as used herein, refers to the act of treating, as “treating” is defined immediately above.

A “therapeutically effective amount” is the amount of an integrin agonist needed to provide a desired level of drug in the tissues, bloodstream, or other physical compartment of a patient, the desired level giving rise to an anticipated physiological response or biological effect when the integrin agonist is administered by the chosen route of administration. The precise amount will depend upon numerous factors including, for example, the particular integrin agonist; the specific pharmaceutical formulation or delivery device employed; the severity of the disease state; and patient adherence to a treatment regimen. Therapeutically effective amounts of integrin agonists can be readily determined by one skilled in the art based upon the information provided herein.

“About” and “around,” as used herein to modify a numerical value, indicate a defined range around that value. If “X” were the value, “about X” or “around X” would generally indicate a value from 0.95X to 1.05X including, for example, from 0.98X to 1.02X or from 0.99X to 1.01X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.” When the quantity “X” only includes whole-integer values (e.g., “X carbons”), “about X” or “around X” indicates from (X−1) to (X+1). In such cases, “about X” or “around X” specifically indicates at least the values X, X−1, and X+1.

### II. INTEGRIN AGONISTS

The present invention provides novel integrin agonists and improved methods for treating β2 integrin-mediated conditions. Compounds provided herein increase the adhesion and functional properties of β2 integrin-expressing cells, including leukocytes such as neutrophils, macrophages, and other myeloid cells, as well as microglia. Such cells can be present in the circulation or resident in tissues or tumors. Reducing recruitment of inflammatory immune cells into tissues by increasing cell adhesion allows for the treatment of acute inflammation, chronic inflammation, cancer, neurological conditions, and other diseases. Additionally, integrin activation affects various cellular functions, such as phagocytosis and signaling pathways. Certain oxazolidine compounds and picolinate-containing thiazolidine compounds are particularly advantageous for activating integrins and increasing integrin-mediated cell adhesion, as described in more detail below.

In one embodiment, the invention provides a compound according to Formula I:

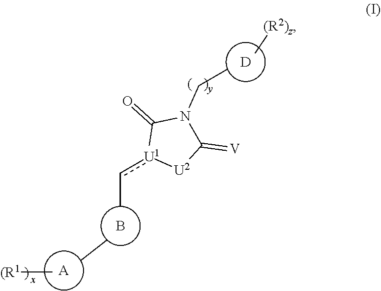

or a pharmaceutically acceptable salt thereof, wherein:
ring A is selected from phenyl and C₆ heteroaryl;
ring B is selected from phenylene and C₅₋₆ heteroarylene, each of which is optionally substituted with one or two C₁₋₄ alkyl;
ring D is selected from cyclohexyl and phenyl;
V is selected from O and S;
U¹ is selected from C, CH, and N;
U² is selected from O, C(R³)(R⁴), and NR^{a};
each R¹ is independently selected from halogen, (CH₂)_{w}COOR^{1a}, and C₅₋₆ heteroaryl, wherein C₅₋₆ heteroaryl is optionally substituted with (CH₂)_{w}COOR^{1a};
each R^{1a} is independently selected from H and C₁₋₆ alkyl;
each subscript w is 0, 1, or 2;
each R² is independently selected from halogen, C₁₋₆ alkyl, C₁₋₆ alkoxy, and R^{2a};
each R^{2a} is independently selected from C₂₋₆ alkyl and C₂₋₆ alkoxy;
each of R³ and R⁴ is independently selected from H and C₁₋₄ alkyl;
R^{a} is selected from H and C₁₋₄ alkyl;
subscript x is 0, 1, 2, 3, 4, or 5;
subscript y is 0, 1, or 2;
subscript z is 0, 1, 2, 3, 4, or 5; and
the dashed line represents a single bond or a double bond;
provided that when V is S, U¹ is C, U² is NH, the dashed line represents a double bond, ring B is furan-2,5-diyl, and (R′)ₓ-(ring A)- is 4-carboxyphenyl,
(R²)_{z}-(ring D)-(CH₂)_{y}— is selected from 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-benzyl, and (R^{2a})_{z}-cyclohexylmethyl, wherein subscript z is 1, 2, or 3;
provided that when V is S, U¹ is C, U² is NH, the dashed line represents a double bond, ring B is furan-2,5-diyl, and (R¹)ₓ-(ring A)- is 4-(COOEt)phenyl,
(R²)_{z}-(ring D)-(CH₂)_{y}— is selected from 4-chlorophenyl, 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-benzyl, and (R^{2a})_{z}-cyclohexylmethyl, wherein subscript z is 1, 2, or 3;
provided that when V is O, U¹ is C, U² is NH, the dashed line represents a double bond, ring B is furan-2,5-diyl, and (R¹)ₓ-(ring A)- is 4-carboxyphenyl,

R²)_{z}-(ring D)-(CH₂)_{y}— is selected from 4-fluorophenyl, 4-methoxyphenyl, 4-methylphenyl, unsubstituted phenethyl, unsubstituted cyclohexymethyl, 3-chlorobenzyl, -2-chlorobenzyl, 3,4-dichlorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{2a})_{z}-phenyl, (R^{2a})_{z}-benzyl, and (R^{2a})_{z}-cyclohexylmethyl, wherein subscript z is 1, 2, or 3; and
provided that when V is O, U¹ is C, U² is S, the dashed line represents a double bond, ring B is furan-2,5-diyl, and (R¹)ₓ-(ring A)- is 4-(COOMe)phenyl or 4-(COOEt)phenyl, R²)_{z}-(ring D)-(CH₂)_{y}— is selected from 4-chlorophenyl, 4-fluorophenyl, 4-methoxyphenyl, 4-methylphenyl, unsubstituted phenethyl, unsubstituted cyclohexymethyl, 4-chlorobenzyl, 3-chlorobenzyl, 2-chlorobenzyl, 3,4-dichlorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-phenyl, (R^{2a})_{z}-benzyl, and (R^{2a})_{z}-cyclohexylmethyl, wherein subscript z is 1, 2, or 3.

In some embodiments, the invention provides compound of Formula I wherein ring B is selected from meta-phenylene; thiophene-2,5-diyl; pyridine-2,6-diyl; pyrimidine-2,4-diyl; and furan-2,5-diyl.

In some embodiments, the invention provides compound of Formula I wherein ring B is selected from meta-phenylene; thiophene-2,5-diyl; pyridine-2,6-diyl; pyrimidine-2,4-diyl; and furan-2,5-diyl and ring D is phenyl. In some embodiments, the invention provides compound of Formula I wherein ring B is selected from meta-phenylene; thiophene-2,5-diyl; pyridine-2,6-diyl; pyrimidine-2,4-diyl; and furan-2,5-diyl, ring D is phenyl, and subscript y is 0. In some embodiments, the invention provides compound of Formula I wherein ring B is selected from meta-phenylene; thiophene-2,5-diyl; pyridine-2,6-diyl; pyrimidine-2,4-diyl; and furan-2,5-diyl, ring D is phenyl, and subscript y is 1. In some embodiments, the invention provides compound of Formula I wherein ring B is selected from meta-phenylene; thiophene-2,5-diyl; pyridine-2,6-diyl; pyrimidine-2,4-diyl; and furan-2,5-diyl, ring D is phenyl, and subscript y is 2.

In some embodiments, the invention provides compound of Formula I wherein ring B is selected from meta-phenylene; thiophene-2,5-diyl; pyridine-2,6-diyl; pyrimidine-2,4-diyl; and furan-2,5-diyl and ring D is cyclohexyl. In some embodiments, the invention provides compound of Formula I wherein ring B is selected from meta-phenylene; thiophene-2,5-diyl; pyridine-2,6-diyl; pyrimidine-2,4-diyl; and furan-2,5-diyl, ring D is cyclohexyl, and subscript y is 0. In some embodiments, the invention provides compound of Formula I wherein ring B is selected from meta-phenylene; thiophene-2,5-diyl; pyridine-2,6-diyl; pyrimidine-2,4-diyl; and furan-2,5-diyl, ring D is cyclohexyl, and subscript y is 1. In some embodiments, the invention provides compound of Formula I wherein ring B is selected from meta-phenylene; thiophene-2,5-diyl; pyridine-2,6-diyl; pyrimidine-2,4-diyl; and furan-2,5-diyl, ring D is cyclohexyl, and subscript y is 2.

In some embodiments, the invention provides compounds of Formula I wherein V is S, U¹ is C, U² is NH, the dashed line represents a double bond, ring B is furan-2,5-diyl, (R¹)ₓ-(ring A)- is 4-carboxyphenyl, and (R²)_{z}-(ring D)-(CH₂)_{y}— is selected from 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, and 2,3-dimethylbenzyl.

In some embodiments, the invention provides compounds of Formula I wherein V is S, U¹ is C, U² is NH, the dashed line represents a double bond, ring B is furan-2,5-diyl, (R¹)ₓ(ring A)- is 4-carboxyphenyl, and (R²)_{z}-(ring D)-(CH₂)_{y}— is selected from (R^{2a})_{z}-benzyl, and (R^{2a})_{z}-cyclohexylmethyl, wherein subscript z is 1, 2, or 3. In some embodiments, z is 1 and R^{2a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 2 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 3 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula I wherein V is S, U¹ is C, U² is NH, the dashed line represents a double bond, ring B is furan-2,5-diyl, (R¹)ₓ(ring A)- is 4-(COOEt)phenyl, and (R²)_{z}-(ring D)-(CH₂)_{y}— is selected from 4-chlorophenyl, 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, and 2,3-dimethylbenzyl.

In some embodiments, the invention provides compounds of Formula I wherein V is S, U¹ is C, U² is NH, the dashed line represents a double bond, ring B is furan-2,5-diyl, (R¹)ₓ(ring A)- is 4-(COOEt)phenyl, and (R²)_{z}-(ring D)-(CH₂)_{y}— is selected from (R^{2a})_{z}-benzyl, and (R^{2a})_{z}-cyclohexylmethyl, wherein subscript z is 1, 2, or 3. In some embodiments, z is 1 and R^{2a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 2 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 3 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula I wherein V is O, U¹ is C, U² is NH, the dashed line represents a double bond, ring B is furan-2,5-diyl, (R¹)ₓ(ring A)- is 4-carboxyphenyl, and R²)_{z}-(ring D)-(CH₂)_{y}— is selected from 4-fluorophenyl, 4-methoxyphenyl, 4-methylphenyl, unsubstituted phenethyl, unsubstituted cyclohexymethyl, 3-chlorobenzyl, =2-chlorobenzyl, 3,4-dichlorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, and 2,3-dimethylbenzyl.

In some embodiments, the invention provides compounds of Formula I wherein V is O, U¹ is C, U² is NH, the dashed line represents a double bond, ring B is furan-2,5-diyl, (R¹)ₓ(ring A)- is 4-carboxyphenyl, and R²)_{z}-(ring D)-(CH₂)_{y}— is selected from (R^{2a})_{z}-phenyl, (R^{2a})_{z}-benzyl, and (R^{2a})_{z}-cyclohexylmethyl, wherein subscript z is 1, 2, or 3. In some embodiments, z is 1 and R^{2a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 2 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 3 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula I wherein V is O, U¹ is C, U² is S, the dashed line represents a double bond, ring B is furan-2,5-diyl, (R¹)ₓ-(ring A)- is 4-(COOMe)phenyl or 4-(COOEt)phenyl, and R²)_{z}-(ring D)-(CH₂)_{y}— is selected from 4-chlorophenyl, 4-fluorophenyl, 4-methoxyphenyl, 4-methylphenyl, unsubstituted phenethyl, unsubstituted cyclohexymethyl, 4-chlorobenzyl, 3-chlorobenzyl, 2-chlorobenzyl, 3,4-dichlorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, and 2,3-dimethylbenzyl.

In some embodiments, the invention provides compounds of Formula I wherein V is O, U¹ is C, U² is S, the dashed line represents a double bond, ring B is furan-2,5-diyl, (R¹)ₓ-(ring A)- is 4-(COOMe)phenyl or 4-(COOEt)phenyl, and R²)_{z}-(ring D)-(CH₂)_{y}— is selected from (R^{2a})_{z}-phenyl, (R^{2a})_{z}-benzyl, and (R^{2a})_{z}-cyclohexylmethyl, wherein subscript z is 1, 2, or 3. In some embodiments, z is 1 and R^{2a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 2 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, z is 3 and each R^{2a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides a compound having a structure according to Formula II

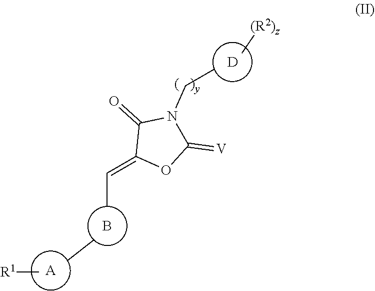

or a pharmaceutically acceptable salt thereof, wherein
R¹ is COOR^{1a}, wherein R^{1a} is selected from H and C₁₋₆ alkyl; and
each R² is independently selected from halogen, C₁₋₆ alkyl, and C₁₋₆ alkoxy.

In some embodiments, ring B in the compound of Formula II is selected from meta-phenylene; thiophene-2,5-diyl; pyridine-2,6-diyl; pyrimidine-2,4-diyl; and furan-2,5-diyl. In some embodiments, ring A in the compound of Formula II is selected from phenyl, pyridin-2-yl, and pyridin-3-yl.

In some embodiments, the invention provides a compound of Formula II wherein ring A is phenyl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1. In some such embodiments, ring D is phenyl substituted with one fluoro, one chloro, or one bromo, and subscript y is 1. In some such embodiments, ring D is phenyl substituted with one fluoro, and subscript y is 1.

In some embodiments, the invention provides a compound of Formula II wherein ring A is pyridin-2-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1. In some such embodiments, ring D is phenyl substituted with one fluoro, one chloro, or one bromo, and subscript y is 1. In some such embodiments, ring D is phenyl substituted with one fluoro, and subscript y is 1.

In some embodiments, the invention provides a compound of Formula II wherein ring A is pyridin-3-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1. In some such embodiments, ring D is phenyl substituted with one fluoro, one chloro, or one bromo, and subscript y is 1. In some such embodiments, ring D is phenyl substituted with one fluoro, and subscript y is 1.

In some embodiments, R^{1a} is H in compounds of Formula II. In some embodiments, R^{1a} is C₁₋₆ alkyl in compounds of Formula II. In some such embodiments, R^{1a} is selected from methyl, ethyl, n-propyl, isopropyl, n-butyl, sec-butyl, and tent-butyl. In some embodiments, R^{1a} is selected from COOH and COOMe.

In some embodiments, the compound is selected from

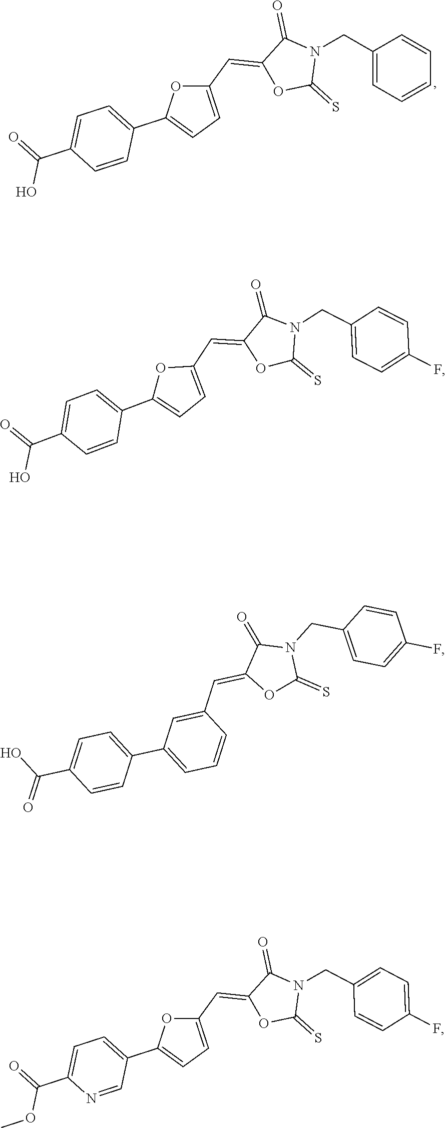 [SMILES: O=C(O)c1ccc(-c2ccc(/C=C3\OC(=S)N(Cc4ccccc4)C3=O)o2)cc1 ; O=C(O)c1ccc(-c2ccc(/C=C3\OC(=S)N(Cc4ccc(F)cc4)C3=O)o2)cc1 ; O=C(O)c1ccc(-c2cccc(/C=C3\OC(=S)N(Cc4ccc(F)cc4)C3=O)c2)cc1 ; COC(=O)c1ccc(-c2ccc(/C=C3\OC(=S)N(Cc4ccc(F)cc4)C3=O)o2)cn1]

and pharmaceutically acceptable salts thereof.

In some embodiments, the invention provides a compound having a structure according to Formula III

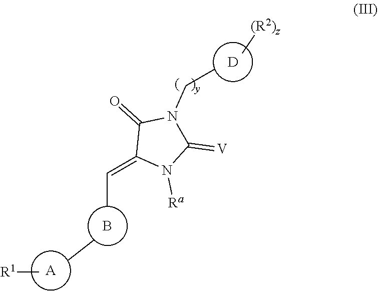

or a pharmaceutically acceptable salt thereof, wherein
R¹ is COOR^{1a}, wherein R^{1a} is selected from H and C₁₋₆ alkyl; and
each R² is independently selected from halogen, C₁₋₆ alkyl, and C₁₋₆ alkoxy.

In some embodiments, R^{a} is H. In some embodiments, R^{a} is selected from methyl, ethyl, n-propyl, n-butyl, sec-butyl, and tent-butyl.

In some embodiments, ring B in the compound of Formula III is selected from meta-phenylene; thiophene-2,5-diyl; pyridine-2,6-diyl; pyrimidine-2,4-diyl; and furan-2,5-diyl. In some embodiments, ring A in the compound of Formula III is selected from phenyl, pyridin-2-yl, and pyridin-3-yl.

In some embodiments, the invention provides a compound of Formula III wherein ring A is phenyl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula III wherein ring A is pyridin-2-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula III wherein ring A is pyridin-3-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, R^{1a} is H in compounds of Formula III. In some embodiments, R^{1a} is C₁₋₆ alkyl in compounds of Formula III. In some such embodiments, R^{1a} is selected from methyl, ethyl, n-propyl, isopropyl, n-butyl, sec-butyl, and tent-butyl. In some embodiments, R^{1a} is selected from COOH and COOMe.

In some embodiments, the invention provides a compound having a structure according to Formula IIIa

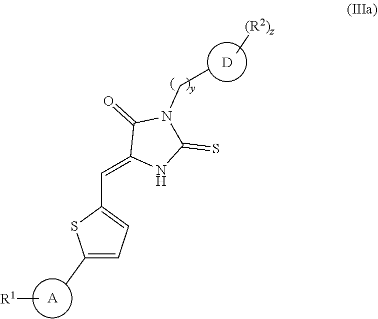

or a pharmaceutically acceptable salt thereof, wherein
R¹ is COOR^{1a}, wherein R^{1a} is selected from H and C₁₋₆ alkyl; and
each R² is independently selected from halogen, C₁₋₆ alkyl, and C₁₋₆ alkoxy.

In some embodiments, ring B in the compound of Formula IIIa is selected from meta-phenylene; thiophene-2,5-diyl; pyridine-2,6-diyl; pyrimidine-2,4-diyl; and furan-2,5-diyl. In some embodiments, ring A in the compound of Formula IIIa is selected from phenyl, pyridin-2-yl, and pyridin-3-yl.

In some embodiments, the invention provides a compound of Formula IIIa wherein ring A is phenyl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula IIIa wherein ring A is pyridin-2-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula IIIa wherein ring A is pyridin-3-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, R^{1a} is H in compounds of Formula IIIa. In some embodiments, R^{1a} is C₁₋₆ alkyl in compounds of Formula IIIa. In some such embodiments, R^{1a} is selected from methyl, ethyl, n-propyl, isopropyl, n-butyl, sec-butyl, and tent-butyl. In some embodiments, R^{1a} is selected from COOH and COOMe.

In some embodiments, the invention provides a compound having a structure according to Formula IIIb:

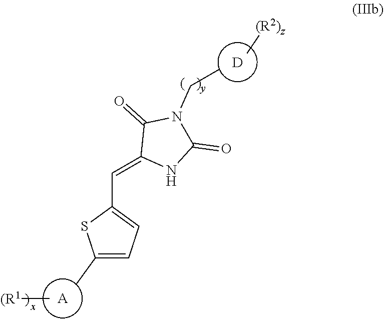

or a pharmaceutically acceptable salt thereof, wherein
R¹ is COOR^{1a}, wherein R^{1a} is selected from H and C₁₋₆ alkyl; and
each R² is independently selected from halogen, C₁₋₆ alkyl, and C₁₋₆ alkoxy.

In some embodiments, ring B in the compound of Formula IIIb is selected from meta-phenylene; thiophene-2,5-diyl; pyridine-2,6-diyl; pyrimidine-2,4-diyl; and furan-2,5-diyl. In some embodiments, ring A in the compound of Formula IIIb is selected from phenyl, pyridin-2-yl, and pyridin-3-yl.

In some embodiments, the invention provides a compound of Formula IIIb wherein ring A is phenyl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula IIIb wherein ring A is pyridin-2-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula IIIb wherein ring A is pyridin-3-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, R^{1a} is H in compounds of Formula IIIb. In some embodiments, R^{1a} is C₁₋₆ alkyl in compounds of Formula IIIb. In some such embodiments, R^{1a} is selected from methyl, ethyl, n-propyl, isopropyl, n-butyl, sec-butyl, and tent-butyl. In some embodiments, R^{1a} is selected from COOH and COOMe.

In some embodiments, the compound is selected from

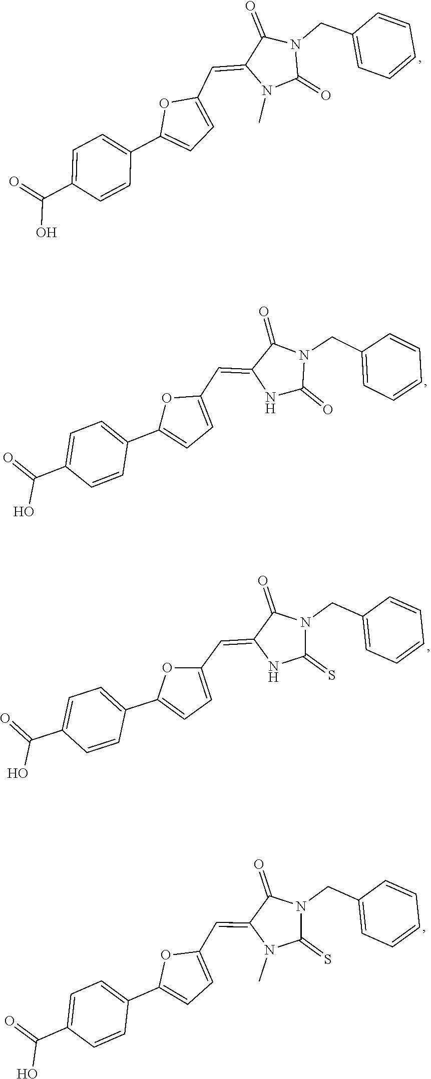 [SMILES: CN1C(=O)N(Cc2ccccc2)C(=O)/C1=C/c1ccc(-c2ccc(C(=O)O)cc2)o1 ; O=C(O)c1ccc(-c2ccc(/C=C3\NC(=O)N(Cc4ccccc4)C3=O)o2)cc1 ; O=C(O)c1ccc(-c2ccc(/C=C3\NC(=S)N(Cc4ccccc4)C3=O)o2)cc1 ; CN1C(=S)N(Cc2ccccc2)C(=O)/C1=C/c1ccc(-c2ccc(C(=O)O)cc2)o1]

and pharmaceutically acceptable salts thereof.

In some embodiments, the invention provides a compound having a structure according to Formula IV

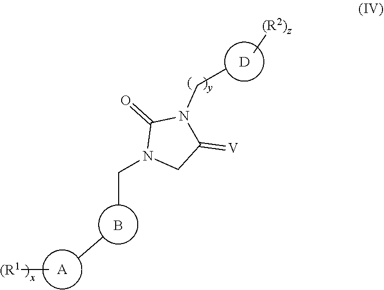

or a pharmaceutically acceptable salt thereof, wherein
R¹ is COOR^{1a}, wherein R^{1a} is selected from H and C₁₋₆ alkyl; and each R² is independently selected from halogen, C₁₋₆ alkyl, and C₁₋₆ alkoxy.

In some embodiments, ring B in the compound of Formula IV is selected from meta-phenylene; thiophene-2,5-diyl; pyridine-2,6-diyl; pyrimidine-2,4-diyl; and furan-2,5-diyl. In some embodiments, ring A in the compound of Formula IV is selected from phenyl, pyridin-2-yl, and pyridin-3-yl.

In some embodiments, the invention provides a compound of Formula IV wherein ring A is phenyl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula IV wherein ring A is pyridin-2-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, the invention provides a compound of Formula IV wherein ring A is pyridin-3-yl and ring B is selected from meta-phenylene; thiophene-2,5-diyl; pyridine-2,6-diyl; pyrimidine-2,4-diyl; and furan-2,5-diyl. In some such embodiments, ring D is cyclohexyl. In some such embodiments, ring D is phenyl. In some such embodiments, ring D is phenyl and subscript y is 1.

In some embodiments, R^{1a} is H in compounds of Formula IV. In some embodiments, R^{1a} is C₁₋₆ alkyl in compounds of Formula IV. In some such embodiments, R^{1a} is selected from methyl, ethyl, n-propyl, isopropyl, n-butyl, sec-butyl, and tent-butyl. In some embodiments, R^{1a} is selected from COOH and COOMe.

In some embodiments, the compound is selected from

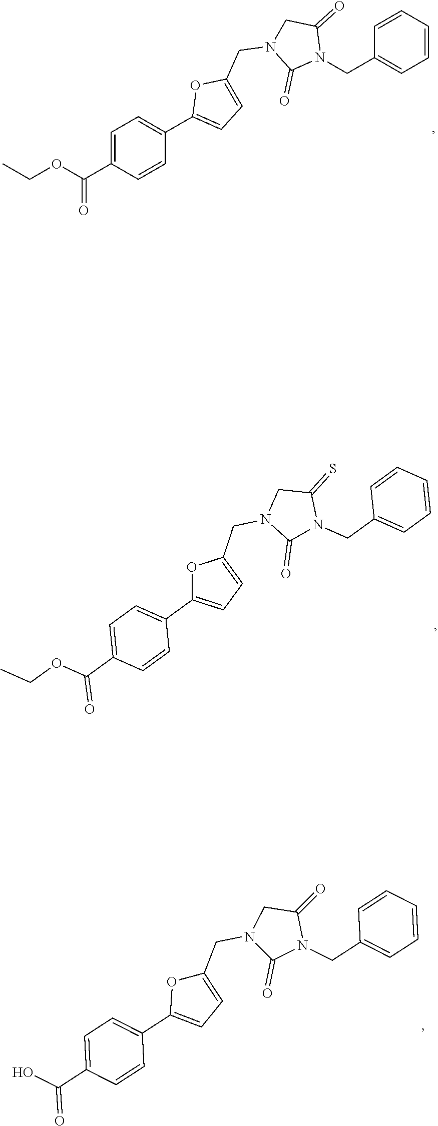 [SMILES: CCOC(=O)c1ccc(-c2ccc(CN3CC(=O)N(Cc4ccccc4)C3=O)o2)cc1 ; CCOC(=O)c1ccc(-c2ccc(CN3CC(=S)N(Cc4ccccc4)C3=O)o2)cc1 ; O=C(O)c1ccc(-c2ccc(CN3CC(=O)N(Cc4ccccc4)C3=O)o2)cc1]

and pharmaceutically acceptable salts thereof.

In some embodiments, the invention provides a compound according to Formula I:

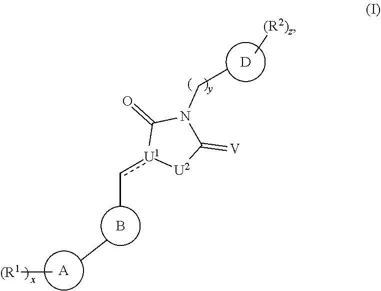

or a pharmaceutically acceptable salt thereof, wherein:
ring A is selected from phenyl and C₆ heteroaryl,
ring B is selected from phenylene and C₅₋₆ heteroarylene, each of which is optionally substituted with one or two C₁₋₄ alkyl;
ring D is selected from cyclohexyl and phenyl;
V is selected from O and S;
U¹ is selected from C, CH, and N;
U² is selected from O, C(R³)(R⁴), and NW;
each R¹ is independently selected from halogen, (CH₂)_{w}COOR^{1a}, and C₅₋₆ heteroaryl, wherein C₅₋₆ heteroaryl is optionally substituted with (CH₂)_{w}COOR^{1a},
provided that at least one R¹ is other than halogen;
each R^{1a} is independently selected from H and C₁₋₆ alkyl;
each subscript w is 0, 1, or 2;
each R² is independently selected from halogen, C₁₋₆ alkyl, C₁₋₆ alkoxy, and R^{2a};
each R^{2a} is independently selected from C₂₋₆ alkyl and C₂₋₆ alkoxy
each of R³ and R⁴ is independently selected from H and C₁₋₄ alkyl;
R^{a} is selected from H and C₁₋₄ alkyl;
subscript x is 0, 1, 2, 3, 4, or 5;
subscript y is 0, 1, or 2;
subscript z is 0, 1, 2, 3, 4, or 5; and
the dashed line represents a single bond or a double bond;
provided that when V is S, U¹ is C, U² is NH, the dashed line represents a double bond, ring B is furan-2,5-diyl, and (R¹)ₓ-(ring A)- is 4-carboxyphenyl,
(R²)_{z}-(ring D)-(CH₂)_{y}— is other than unsubstituted cyclohexyl, unsubstituted phenyl, mono-chlorophenyl, 3-chloro-4-methylphenyl, 2-fluorophenyl, 2-methylphenyl, 2-ethylphenyl, 3-ethylphenyl, isopropylphenyl, 2,3-dimethylphenyl, 2,4-dimethylphenyl, 3,4-dimethylphenyl, 3,5-dimethylphenyl, 3-methoxyphenyl, 4-bromobenzyl, or 2-chlorobenzyl;
provided that when V is S, U¹ is C, U² is NH, the dashed line represents a double bond, ring B is furan-2,5-diyl, and (R¹)ₓ-(ring A)- is 4-(COOEt)phenyl,
(R²)_{z}-(ring D)-(CH₂)_{y}— is other than unsubstituted cyclohexyl, unsubstituted phenyl, 2-methylphenyl, 3-methylphenyl, 2-ethylphenyl, 2-methoxyphenyl, 2-ethoxyphenyl, or 3,4-diethoxyphenethyl;
provided that when V is O, U^{1l} is C, U² is NH, the dashed line represents a double bond, ring B is furan-2,5-diyl, and (R¹)ₓ-(ring A)- is 4-carboxyphenyl,
(R²)_{z}-(ring D)-(CH₂)_{y}— is other than 4-chlorophenyl, 3-chlorobenzyl, 4-chlorobenzyl, 2-fluorobenzyl, 4-fluorobenzyl, or 4-methylbenzyl;
provided that when V is O, U¹ is C, U² is S, the dashed line represents a double bond, ring B is furan-2,5-diyl, and (R¹)ₓ-(ring A)- is 4-(COOMe)phenyl,
(R²)_{z}-(ring D)-(CH₂)_{y}— is other than unsubstituted phenyl, 3-chlorophenyl, or 4-fluorobenzyl; and
provided that when V is O, U¹ is C, U² is NH, the dashed line represents a double bond, ring B is furan-2,5-diyl, and (R¹)ₓ-(ring A)- is 4-(COOEt)phenyl, (R²)_{z}-(ring D)-(CH₂)_{y}— is other than unsubstituted phenyl, 3-chlorophenyl, 2-fluorobenzyl, or 4-fluorobenzyl.

In another embodiment, the invention provides a compound according to Formula XI:

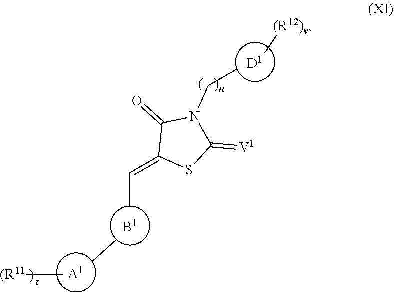

or a pharmaceutically acceptable salt thereof, wherein:
ring A¹ is C₆ heteroaryl;
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen and (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R^{12a};
each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4, or 5.

In some embodiments, the invention provides a compound having a structure according to Formula XIIa

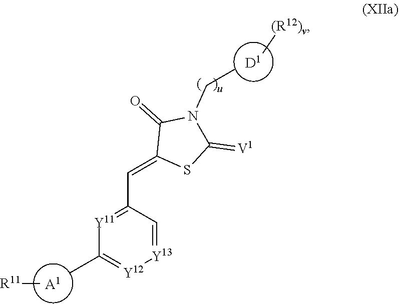

or a pharmaceutically acceptable salt thereof, wherein
R¹¹ is COOR^{11a};
R^{11a} is selected from the group consisting of H and C₁₋₆ alkyl;
each R¹² is independently selected from the group consisting of halogen, C₁₋₆ alkyl, and C₁₋₆ alkoxy; and
Y¹¹, Y¹², and Y¹³ are independently selected from the group consisting of CH and N.

In some embodiments, the invention provides compounds of Formula XIIa wherein Y¹¹, Y¹², and Y¹³ are CH. In some embodiments, Y¹¹ is N, and Y¹² and Y¹³ are CH. In some embodiments, Y¹¹ and Y¹² are N, and Y¹³ is CH.

In some embodiments, R^{11a} is H in compounds of Formula XIIa. In some embodiments, R^{11a} is C₁₋₆ alkyl in compounds of Formula XIIa. In some such embodiments, R^{11a} is selected from methyl, ethyl, n-propyl, isopropyl, n-butyl, sec-butyl, and tent-butyl.

In some embodiments, the invention provides compounds of Formula XIIa wherein R^{11a} is selected from COOH and COOMe. In some embodiments the moiety (R¹¹)-(ring A¹) in the compound of Formula XIIa is selected from 6-carboxy-pyridin-3-yl, 6-methoxycarbonyl-pyridin-3-yl, 5-carboxy-pyridin-2-yl, and 5-methoxycarbonyl-pyridin-2-yl.

In some embodiments, the invention provides compounds of Formula XIIa, wherein subscript u is 0 and subscript v is 0, 1, 2, or 3. In some embodiments, the invention provides compounds of Formula XIIa, wherein subscript u is 1 and subscript v is 0, 1, 2, or 3. In some embodiments, the invention provides compounds of Formula XIIa, wherein subscript u is 2 and subscript v is 0, 1, 2, or 3. In some embodiments, the invention provides compounds of Formula XIIa, wherein subscript u is 0, 1, or 2 and subscript v is 0. In some embodiments, the invention provides compounds of Formula XIIa, wherein subscript u is 0, 1, or 2 and subscript v is 1. In some embodiments, the invention provides compounds of Formula XIIa, wherein subscript u is 0, 1, or 2 and subscript v is 2. In some embodiments, the invention provides compounds of Formula XIIa, wherein subscript u is 0, 1, or 2 and subscript v is 3.

In some embodiments, the invention provides a compound having a structure according to Formula XIIb:

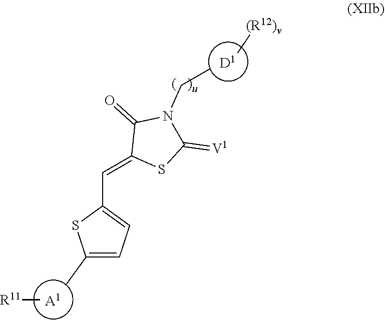

or a pharmaceutically acceptable salt thereof, wherein
R¹¹ is COOR^{11a};
R^{11a} is selected from H and C₁₋₆ alkyl; and
each R¹² is independently selected from halogen, C₁₋₆ alkyl, and C₁₋₆ alkoxy.

In some embodiments, the invention provides compounds of Formula XIIb wherein ring A¹ is selected from pyridin-2-yl and pyridin-3-yl.

In some embodiments, R^{11a} is H in compounds of Formula XIIb. In some embodiments, R^{11a} is C₁₋₆ alkyl in compounds of Formula XIIb. In some such embodiments, R^{11a} is selected from methyl, ethyl, n-propyl, isopropyl, n-butyl, sec-butyl, and tent-butyl.

In some embodiments, the invention provides compounds of Formula XIIb wherein R^{11a} is selected from COOH and COOMe. In some embodiments the moiety (R¹¹)-(ring A¹) in the compound of Formula XIIb is selected from 6-carboxy-pyridin-3-yl, 6-methoxycarbonyl-pyridin-3-yl, 5-carboxy-pyridin-2-yl, and 5-methoxycarbonyl-pyridin-2-yl.

In some embodiments, the invention provides compounds of Formula XIIb, wherein subscript u is 0 and subscript v is 0, 1, 2, or 3. In some embodiments, the invention provides compounds of Formula XIIb, wherein subscript u is 1 and subscript v is 0, 1, 2, or 3. In some embodiments, the invention provides compounds of Formula XIIb, wherein subscript u is 2 and subscript v is 0, 1, 2, or 3. In some embodiments, the invention provides compounds of Formula XIIb, wherein subscript u is 0, 1, or 2 and subscript v is 0. In some embodiments, the invention provides compounds of Formula XIIb, wherein subscript u is 0, 1, or 2 and subscript v is 1. In some embodiments, the invention provides compounds of Formula XIIb, wherein subscript u is 0, 1, or 2 and subscript v is 2. In some embodiments, the invention provides compounds of Formula XIIb, wherein subscript u is 0, 1, or 2 and subscript v is 3.

In some embodiments, the invention provides a compound having a structure according to Formula XIIc:

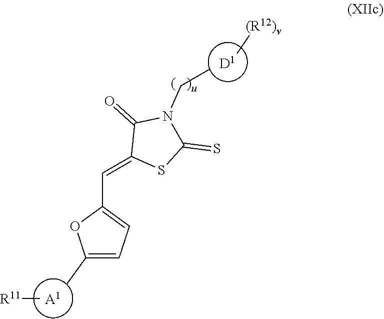

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

In some embodiments, ring A¹ is selected from pyridin-2-yl and pyridin-3-yl.

In some embodiments, R^{11a} is H in compounds of Formula XIIc. In some embodiments, R^{11a} is C₁₋₆ alkyl in compounds of Formula XIIc. In some such embodiments, R^{11a} is selected from methyl, ethyl, n-propyl, isopropyl, n-butyl, sec-butyl, and tent-butyl.

In some embodiments, the invention provides compounds of Formula XIIc wherein R^{11a} is selected from COOH and COOMe. In some embodiments, the moiety (R¹¹)-(ring A¹) in the compound of Formula XIIc is selected from 6-carboxy-pyridin-3-yl, 6-methoxycarbonyl-pyridin-3-yl, 5-carboxy-pyridin-2-yl, and 5-methoxycarbonyl-pyridin-2-yl.

In some embodiments, the invention provides compounds of Formula XIIc, wherein subscript u is 0 and subscript v is 0, 1, 2, or 3. In some embodiments, the invention provides compounds of Formula XIIc, wherein subscript u is 1 and subscript v is 0, 1, 2, or 3. In some embodiments, the invention provides compounds of Formula XIIc, wherein subscript u is 2 and subscript v is 0, 1, 2, or 3. In some embodiments, the invention provides compounds of Formula XIIc, wherein subscript u is 0, 1, or 2 and subscript v is 0. In some embodiments, the invention provides compounds of Formula XIIc, wherein subscript u is 0, 1, or 2 and subscript v is 1. In some embodiments, the invention provides compounds of Formula XIIc, wherein subscript u is 0, 1, or 2 and subscript v is 2. In some embodiments, the invention provides compounds of Formula XIIc, wherein subscript u is 0, 1, or 2 and subscript v is 3.

In some embodiments, the compound is selected from:

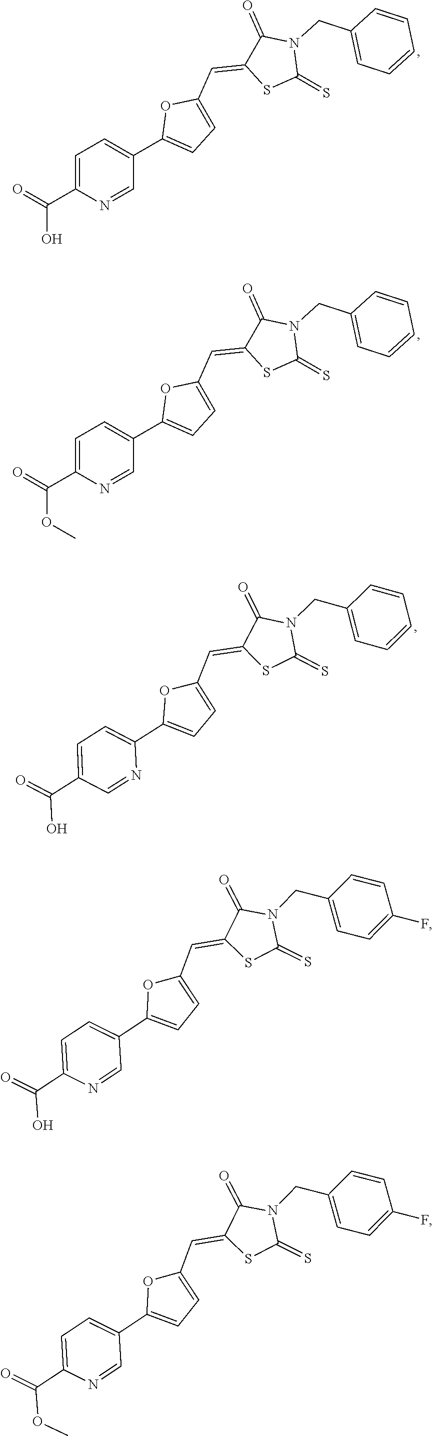 [SMILES: O=C(O)c1ccc(-c2ccc(/C=C3\SC(=S)N(Cc4ccccc4)C3=O)o2)cn1 ; COC(=O)c1ccc(-c2ccc(/C=C3\SC(=S)N(Cc4ccccc4)C3=O)o2)cn1 ; O=C(O)c1ccc(-c2ccc(/C=C3\SC(=S)N(Cc4ccccc4)C3=O)o2)nc1 ; O=C(O)c1ccc(-c2ccc(/C=C3\SC(=S)N(Cc4ccc(F)cc4)C3=O)o2)cn1 ; COC(=O)c1ccc(-c2ccc(/C=C3\SC(=S)N(Cc4ccc(F)cc4)C3=O)o2)cn1]

and pharmaceutically acceptable salts thereof.

In some embodiments, the invention provides a compound having a structure according to Formula XIId:

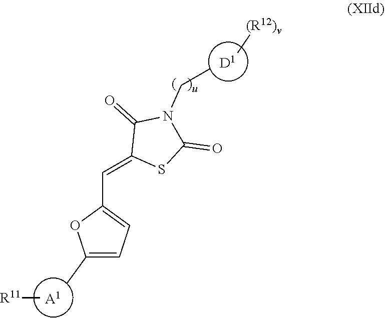

or a pharmaceutically acceptable salt thereof, wherein
R¹¹ is COOR^{11a};
R^{11a} is selected from H and C₁₋₆ alkyl; and
each R¹² is independently selected from halogen, C₁₋₆ alkyl, and C₁₋₆ alkoxy.

In some embodiments, ring A¹ is selected from pyridin-2-yl and pyridin-3-yl.

In some embodiments, R^{11a} is H in compounds of Formula XIId. In some embodiments, R^{11a} is C₁₋₆ alkyl in compounds of Formula XIId. In some such embodiments, R^{11a} is selected from methyl, ethyl, n-propyl, isopropyl, n-butyl, sec-butyl, and tent-butyl.

In some embodiments, the invention provides compounds of Formula XIId wherein R^{11a} is selected from COOH and COOMe. In some embodiments, the moiety (R¹¹)-(ring A¹) in the compound of Formula XIId is selected from 6-carboxy-pyridin-3-yl, 6-methoxycarbonyl-pyridin-3-yl, 5-carboxy-pyridin-2-yl, and 5-methoxycarbonyl-pyridin-2-yl.

In some embodiments, the invention provides compounds of Formula XIId, wherein subscript u is 0 and subscript v is 0, 1, 2, or 3. In some embodiments, the invention provides compounds of Formula XIId, wherein subscript u is 1 and subscript v is 0, 1, 2, or 3. In some embodiments, the invention provides compounds of Formula XIId, wherein subscript u is 2 and subscript v is 0, 1, 2, or 3. In some embodiments, the invention provides compounds of Formula XIId, wherein subscript u is 0, 1, or 2 and subscript v is 0. In some embodiments, the invention provides compounds of Formula XIId, wherein subscript u is 0, 1, or 2 and subscript v is 1. In some embodiments, the invention provides compounds of Formula XIId, wherein subscript u is 0, 1, or 2 and subscript v is 2. In some embodiments, the invention provides compounds of Formula XIId, wherein subscript u is 0, 1, or 2 and subscript v is 3.

In another embodiment, the invention provides a compound according to Formula XIII:

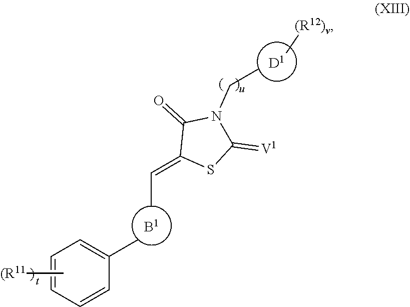

or a pharmaceutically acceptable salt thereof, wherein:
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen, (CH₂)ₛCOOR^{11a}, and C₅₋₆ heteroaryl, wherein C₅₋₆ heteroaryl is optionally substituted with (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R^{12a};
each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4, or 5;
provided that when V¹ is O, ring B¹ is phen-1,5-diyl, R¹¹ is 4-carboxy, and subscript t is 1,
(R¹²)ᵥ-(ring D¹)-(CH₂)ᵤ— is other than 4-methoxybenzyl;
provided that when V¹ is S, ring B¹ is thiophen-2,5-diyl, R¹¹ is 4-(COOMe), and subscript t is 1,
(R¹²)ᵥ-(ring D¹)-(CH₂)ᵤ— is other than 3,4,5-trimethoxybenzyl;
provided that when V¹ is S, ring B¹ is furan-2,5-diyl, R¹¹ is 4-carboxy, and subscript t is 1,
(R¹²)ᵥ-(ring D¹)-(CH₂)ᵤ— is selected from the group consisting of unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{12a})ᵥ-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOMe), and subscript t is 1,
(R¹²)ᵥ-(ring D¹)-(CH₂)ᵤ-is selected from the group consisting of 4-chlorophenyl, 4-fluorophenyl, 4-methylphenyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{12a})_{z}-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOEt), and subscript t is 1,
(R¹²)ᵥ-(ring D¹)-(CH₂)ᵤ— is selected from the group consisting of unsubstituted cyclohexymethyl, 4-chlorobenzyl, 3-chlorobenzyl, 3,4-dichlorobenzyl, 4-isopropylbenzyl, 3-methoxybenzyl, 2-methoxybenzyl, 2-methylbenzyl, 2,4-dimethylbenzyl, 3,4-dimethylbenzyl, 2,3-dimethylbenzyl, (R^{12a})ᵥ-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOnBu), and subscript t is 1,
(R¹²)ᵥ-(ring D¹)-(CH₂)ᵤ— is selected from the group consisting of unsubstituted phenethyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3 -methoxybenzyl, 2-methoxybenzyl, 4-methylbenzyl, 3 -methylbenzyl, 2-methylbenzyl, 2,4-dimethylbenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-carboxy, and subscript t is 1,
(R¹²)ᵥ-(ring D¹)-(CH₂)ᵤ— is selected from the group consisting of 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-(COOMe), and subscript t is 1,
(R¹²)ᵥ-(ring D¹¹)-(CH₂)ᵤ— is selected from the group consisting of 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-(COOEt), and subscript t is 1,
(R¹²)ᵥ-(ring D¹)-(CH₂)ᵤ— is selected from the group consisting of 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, is 4-(COOnBu), and subscript t is 1,
(R¹²)ᵤ-(ring D¹)-(CH₂)ᵤ— is selected from the group consisting of 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3.

In some embodiments, the invention provides compound of Formula XIII wherein ring B¹ is selected from phen-1,5-diyl, thiophen-2,5-diyl, and furan-2,5-diyl.

In some embodiments, the invention provides compounds of Formula XIII wherein V¹ is O, ring B¹ is phen-1,5-diyl, R¹¹ is 4-carboxy, subscript t is 1, and each R¹² is independently selected from halogen, C₁₋₆ alkyl, and C₂₋₆ alkoxy.

In some embodiments, the invention provides compounds of Formula XIII wherein V¹ is S, ring B¹ is thiophen-2,5-diyl, R¹¹ is 4-(COOMe), subscript t is 1, and each R¹² is independently selected from halogen, C₁₋₆ alkyl, and C₂₋₆ alkoxy.

In some embodiments, the invention provides compounds of Formula XIII wherein V¹ is S, ring B¹ is furan-2,5-diyl, R¹¹ is 4-carboxy, subscript t is 1, and (R¹²)ᵥ-(ring D¹)-(CH₂)ᵤ— is selected from unsubstituted cyclohexymethyl, 4-chlorobenzyl, 3-chlorobenzyl, 2-chlorobenzyl, 3 ,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, and 2,3 -dimethylbenzyl.

In some embodiments, the invention provides compounds of Formula XIII wherein V¹ is S, ring B¹ is furan-2,5-diyl, R¹¹ is 4-carboxy, subscript t is 1, and (R¹²)ᵥ-(ring D¹)-(CH₂)ᵤ— is selected from (R^{2a})_{z}-phenyl, (R^{2a})_{z}-benzyl, and (R^{2a})_{z}-cyclohexylmethyl, wherein subscript z is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is S, ring B¹ is furan-2,5-diyl, R¹¹ is 4-(COOMe), subscript t is 1, and (R¹²)ᵥ-(ring D¹)-(CH₂)ᵤ— is selected from 4-chlorophenyl, 4-fluorophenyl, 4-methylphenyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, and 2,3-dimethylbenzyl.

In some embodiments, the invention provides compounds of Formula XIII wherein V¹ is S, ring B¹ is furan-2,5-diyl, R¹¹ is 4-(COOMe), subscript t is 1, and (R¹²)ᵥ-(ring D¹)-(CH₂)ᵤ— is selected from (R^{2a})_{z}-phenyl, (R^{2a})_{z}-benzyl, and (R^{2a})_{z}-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is S, ring B¹ is furan-2,5-diyl, R¹¹ is 4-(COOEt), subscript t is 1, and (R¹²)ᵥ-(ring D¹)-(CH₂)ᵤ— is selected from unsubstituted cyclohexymethyl, 4-chlorobenzyl, 3-chlorobenzyl, 3,4-dichlorobenzyl, 4-isopropylbenzyl, 3 -methoxybenzyl, 2-methoxybenzyl, 2-methylbenzyl, 2,4-dimethylbenzyl, 3,4-dimethylbenzyl, and 2,3-dimethylbenzyl.

In some embodiments, the invention provides compounds of Formula XIII wherein V¹ is S, ring B¹ is furan-2,5-diyl, R¹¹ is 4-(COOEt), subscript t is 1, and (R¹²)ᵥ-(ring D¹)-(CH₂)ᵤ— is selected from (R^{2a})_{z}-phenyl, (R^{2a})_{z}-benzyl, and (R^{2a})_{z}-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is S, ring B¹ is furan-2,5-diyl, R¹¹ is 4-(COOnBu), and subscript t is 1, and (R¹²)ᵥ-(ring D¹)-(CH₂)ᵤ— is selected from unsubstituted phenethyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, and 2,3-dimethylbenzyl.

In some embodiments, the invention provides compounds of Formula XIII wherein V¹ is S, ring B¹ is furan-2,5-diyl, R¹¹ is 4-(COOnBu), subscript t is 1, and (R¹²)ᵥ-(ring D¹)-(CH₂)ᵤ— is selected from (R^{12a})ᵥ-phenyl, (R^{12a})ᵥ-benzyl, and (R^{12a})ᵥ-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is O, ring B¹ is furan-2,5-diyl, R¹¹ is 4-carboxy, and subscript t is 1, and (R¹²)ᵥ-(ring D¹)-(CH₂)ᵤ— is selected from 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, and 2,3-dimethylbenzyl.

In some embodiments, the invention provides compounds of Formula XIII wherein V¹ is O, ring B¹ is furan-2,5-diyl, R¹¹ is 4-carboxy, and subscript t is 1, and (R¹²)ᵥ-(ring D¹)-(CH₂)ᵤ— is selected from (R^{12a})ᵥ-phenyl, (R^{12a})ᵥ-benzyl, and (R^{12a})ᵥ-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is O, ring B¹ is furan-2,5-diyl, R¹¹ is 4-(COOMe), and subscript t is 1, and (R¹²)ᵥ-(ring D¹)-(CH₂)ᵤ— is selected from 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, and 2,3-dimethylbenzyl.

In some embodiments, the invention provides compounds of Formula XIII wherein V¹ is O, ring B¹ is furan-2,5-diyl, R¹¹ is 4-(COOMe), and subscript t is 1, (R¹²)ᵥ-(ring D¹)-(CH₂)ᵤ— is selected from 2,3-dimethylbenzyl, (R^{12a})ᵥ-phenyl, (R^{12a})ᵥ-benzyl, and (R^{12a})^{v}-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is O, ring B¹ is furan-2,5-diyl, R¹¹ is 4-(COOEt), and subscript t is 1, and (R¹²)ᵥ-(ring D¹)-(CH₂)ᵤ— is selected from 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 3,4-dimethylbenzyl, and 2,3-dimethylbenzyl.

In some embodiments, the invention provides compounds of Formula XIII wherein V¹ is O, ring B¹ is furan-2,5-diyl, R¹¹ is 4-(COOEt), and subscript t is 1, and (R¹²)ᵥ-(ring D¹)-(CH₂)ᵤ— is selected from (R^{12a})ᵥ-phenyl, (R^{12a})ᵥ-benzyl, and (R¹²a)ᵥ-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides compounds of Formula XIII wherein V¹ is O, ring B¹ is furan-2,5-diyl, R¹¹ is 4-(COOnBu), and subscript t is 1, and (R¹²)ᵥ-(ring D¹)-(CH₂)ᵤ— is selected from 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, and 2,3-dimethylbenzyl.

In some embodiments, the invention provides compounds of Formula XIII wherein V¹ is O, ring B¹ is furan-2,5-diyl, R¹¹ is 4-(COOnBu), and subscript t is 1, and (R¹²)ᵥ-(ring D¹)-(CH₂)ᵤ— is selected from (R^{12a})ᵥ-phenyl, (R^{12a})ᵥ-benzyl, and (R^{12a})ᵥ-cyclohexylmethyl, wherein subscript v is 1, 2, or 3. In some embodiments, subscript v is 1 and R^{12a} is ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 2 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy. In some embodiments, subscript v is 3 and each R^{12a} is independently ethyl, n-propyl, isopropyl, n-butyl, sec-butyl, tent-butyl, ethoxy, n-propoxy, isopropoxy, n-butoxy, sec-butoxy, or tert-butoxy.

In some embodiments, the invention provides a compound having a structure according to Formula XIVa:

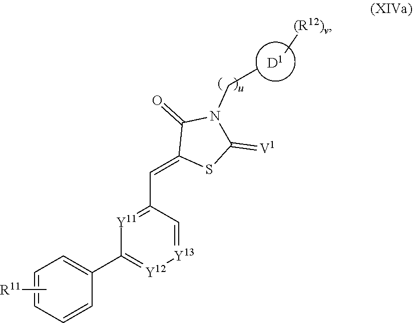

or a pharmaceutically acceptable salt thereof, wherein
R¹¹ is COOR^{11a};
R^{11a} is selected from the group consisting of H and C₁₋₆ alkyl;
each R¹² is independently selected from the group consisting of halogen, C₁₋₆ alkyl, and C₁₋₆ alkoxy; and
Y¹¹, Y¹² and Y¹³ are independently selected from the group consisting of CH and N.

In some embodiments, Y¹¹, Y¹², and Y¹³ are CH in compounds of Formula XIVa. In some embodiments, Y¹¹ is N, and Y¹² and Y¹³ are CH. In some embodiments, Y¹¹ and Y¹² are N, and Y¹³ is CH.

In some embodiments, R^{11a} is H in compounds of Formula XIVa. In some embodiments, R^{11a} is C₁₋₆ alkyl in compounds of Formula XIVa. In some such embodiments, R^{11a} is selected from methyl, ethyl, n-propyl, isopropyl, n-butyl, sec-butyl, and tent-butyl. In some embodiments, R^{11a} is selected from COOH and COOMe.

In some embodiments, the invention provides compounds of Formula XIVa, wherein subscript u is 0 and subscript v is 0, 1, 2, or 3. In some embodiments, the invention provides compounds of Formula XIVa, wherein subscript u is 1 and subscript v is 0, 1, 2, or 3. In some embodiments, the invention provides compounds of Formula XIVa, wherein subscript y is u and subscript v is 0, 1, 2, or 3. In some embodiments, the invention provides compounds of Formula XIVa, wherein subscript u is 0, 1, or 2 and subscript v is 0. In some embodiments, the invention provides compounds of Formula XIVa, wherein subscript u is 0, 1, or 2 and subscript v is 1. In some embodiments, the invention provides compounds of Formula XIVa, wherein subscript u is 0, 1, or 2 and subscript v is 2. In some embodiments, the invention provides compounds of Formula XIVa, wherein subscript u is 0, 1, or 2 and subscript v is 3. In some such embodiments, ring D¹ is phenyl. In some such embodiments, ring D¹ is phenyl and subscript u is 1. In some such embodiments, ring D¹ is phenyl substituted with one fluoro, one chloro, or one bromo, and subscript u is 1. In some such embodiments, ring D¹ is phenyl substituted with one fluoro, and subscript u is 1.

In some embodiments, the compound is selected from:

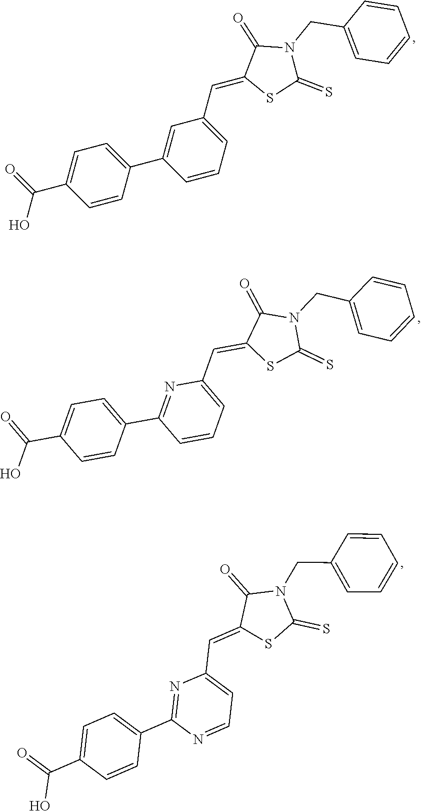 [SMILES: O=C(O)c1ccc(-c2cccc(/C=C3\SC(=S)N(Cc4ccccc4)C3=O)c2)cc1 ; O=C(O)c1ccc(-c2cccc(/C=C3\SC(=S)N(Cc4ccccc4)C3=O)n2)cc1 ; O=C(O)c1ccc(-c2nccc(/C=C3\SC(=S)N(Cc4ccccc4)C3=O)n2)cc1]

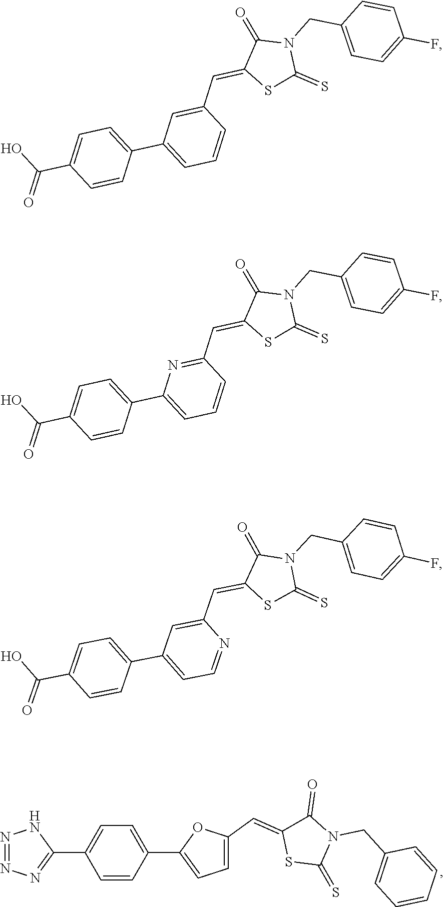 [SMILES: O=C(O)c1ccc(-c2cccc(/C=C3\SC(=S)N(Cc4ccc(F)cc4)C3=O)c2)cc1 ; O=C(O)c1ccc(-c2cccc(/C=C3\SC(=S)N(Cc4ccc(F)cc4)C3=O)n2)cc1 ; O=C(O)c1ccc(-c2ccnc(/C=C3\SC(=S)N(Cc4ccc(F)cc4)C3=O)c2)cc1 ; O=C1/C(=C/c2ccc(-c3ccc(-c4nnn[nH]4)cc3)o2)SC(=S)N1Cc1ccccc1]

and pharmaceutically acceptable salts thereof.

In some embodiments, the compound is selected from:

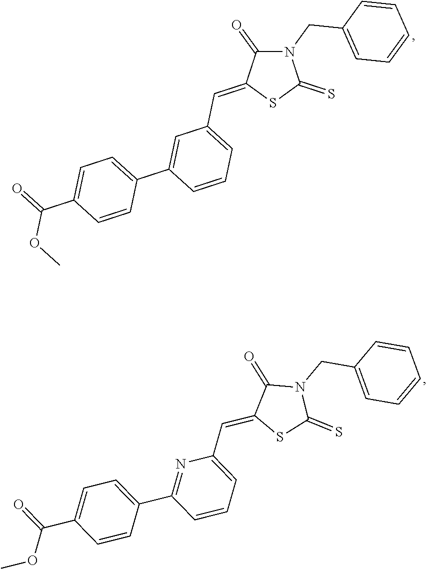 [SMILES: COC(=O)c1ccc(-c2cccc(/C=C3\SC(=S)N(Cc4ccccc4)C3=O)c2)cc1 ; COC(=O)c1ccc(-c2cccc(/C=C3\SC(=S)N(Cc4ccccc4)C3=O)n2)cc1]

and pharmaceutically acceptable salts thereof.

In some embodiments, the invention provides a compound having a structure according to Formula XIVb:

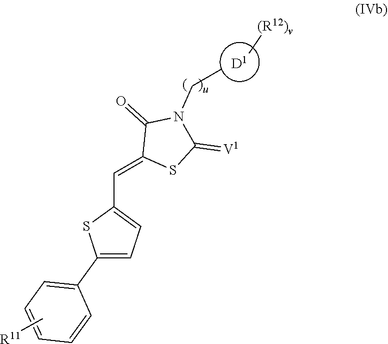

or a pharmaceutically acceptable salt thereof, wherein
R¹¹ is COOR^{11a};
R^{11a} is selected from H and C₁₋₆ alkyl; and
each R¹² is independently selected from halogen, C₁₋₆ alkyl, and C₁₋₆ alkoxy.

In some embodiments, R^{11a} is H in compounds of Formula XIVb. In some embodiments, R^{11a} is C₁₋₆ alkyl in compounds of Formula XIVb. In some such embodiments, R^{11a} is selected from methyl, ethyl, n-propyl, isopropyl, n-butyl, sec-butyl, and tent-butyl. In some embodiments, R^{11a} is selected from COOH and COOMe.

In some embodiments, the invention provides compounds of Formula XIVb, wherein subscript u is 0 and subscript v is 0, 1, 2, or 3. In some embodiments, the invention provides compounds of Formula XIVb, wherein subscript u is 1 and subscript v is 0, 1, 2, or 3. In some embodiments, the invention provides compounds of Formula XIVb, wherein subscript u is 2 and subscript v is 0, 1, 2, or 3. In some embodiments, the invention provides compounds of Formula XIVb, wherein subscript u is 0, 1, or 2 and subscript v is 0. In some embodiments, the invention provides compounds of Formula XIVb, wherein subscript u is 0, 1, or 2 and subscript v is 1. In some embodiments, the invention provides compounds of Formula XIVb, wherein subscript u is 0, 1, or 2 and subscript v is 2. In some embodiments, the invention provides compounds of Formula XIVb, wherein subscript v is 0, 1, or 2 and subscript v is 3. In some such embodiments, ring D¹ is phenyl. In some such embodiments, ring D¹ is phenyl and subscript u is 1.

In some embodiments, the compound is

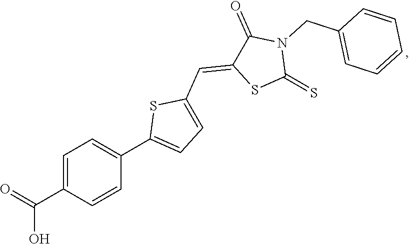 [SMILES: O=C(O)c1ccc(-c2ccc(/C=C3\SC(=S)N(Cc4ccccc4)C3=O)s2)cc1]

or a pharmaceutically acceptable salt thereof.

In some embodiments, the invention provides a compound having a structure according to Formula XIVc:

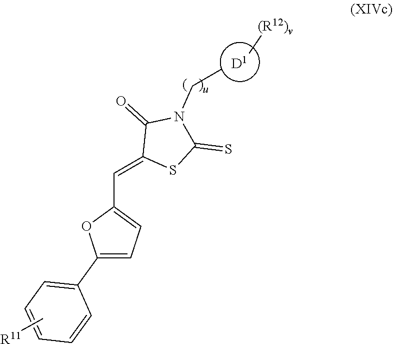

or a pharmaceutically acceptable salt thereof, wherein
R¹¹ is COOR^{11a};
R^{11a} is selected from H and C₁₋₆ alkyl; and
each R¹² is independently selected from halogen, C₁₋₆ alkyl, and C₁₋₆ alkoxy.

In some embodiments, R^{11a} is H in compounds of Formula XIVc. In some embodiments, R^{11a} is C₁₋₆ alkyl in compounds of Formula XIVc. In some such embodiments, R^{11a} is selected from methyl, ethyl, n-propyl, isopropyl, n-butyl, sec-butyl, and tent-butyl. In some embodiments, R^{11a} is selected from COOH and COOMe.

In some embodiments, the invention provides compounds of Formula XIVc, wherein subscript u is 0 and subscript v is 0, 1, 2, or 3. In some embodiments, the invention provides compounds of Formula XIVc, wherein subscript u is 1 and subscript v is 0, 1, 2, or 3. In some embodiments, the invention provides compounds of Formula XIVc, wherein subscript u is 2 and subscript v is 0, 1, 2, or 3. In some embodiments, the invention provides compounds of Formula XIVc, wherein subscript u is 0, 1, or 2 and subscript v is 0. In some embodiments, the invention provides compounds of Formula XIVc, wherein subscript u is 0, 1, or 2 and subscript v is 1. In some embodiments, the invention provides compounds of Formula XIVc, wherein subscript u is 0, 1, or 2 and subscript v is 2. In some embodiments, the invention provides compounds of Formula XIVc, wherein subscript u is 0, 1, or 2 and subscript v is 3. In some such embodiments, ring D¹ is cyclohexyl. In some such embodiments, ring D¹ is cycloxhexyl and subscript u is 1. In some such embodiments, ring D¹ is phenyl. In some such embodiments, ring D¹ is phenyl and subscript u is 1. In some such embodiments, ring D¹ is phenyl substituted with one methyl group, two methyl groups, one isopropyl group, one methoxy group, one hydroxy group, one chloro group, two chloro groups, or one fluoro group, and subscript u is 1.

In some embodiments, the compound is selected from:

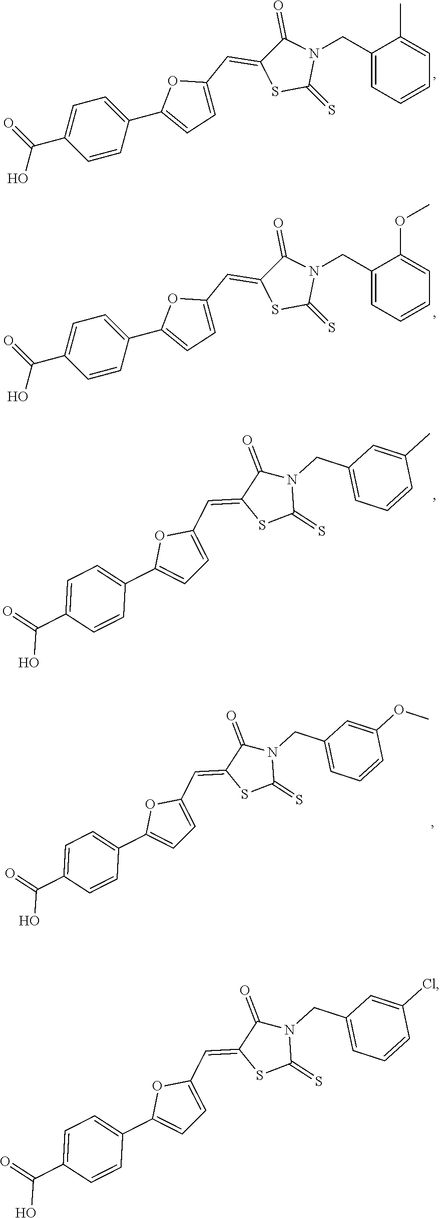 [SMILES: Cc1ccccc1CN1C(=O)/C(=C/c2ccc(-c3ccc(C(=O)O)cc3)o2)SC1=S ; COc1ccccc1CN1C(=O)/C(=C/c2ccc(-c3ccc(C(=O)O)cc3)o2)SC1=S ; Cc1cccc(CN2C(=O)/C(=C/c3ccc(-c4ccc(C(=O)O)cc4)o3)SC2=S)c1 ; COc1cccc(CN2C(=O)/C(=C/c3ccc(-c4ccc(C(=O)O)cc4)o3)SC2=S)c1 ; O=C(O)c1ccc(-c2ccc(/C=C3\SC(=S)N(Cc4cccc(Cl)c4)C3=O)o2)cc1]

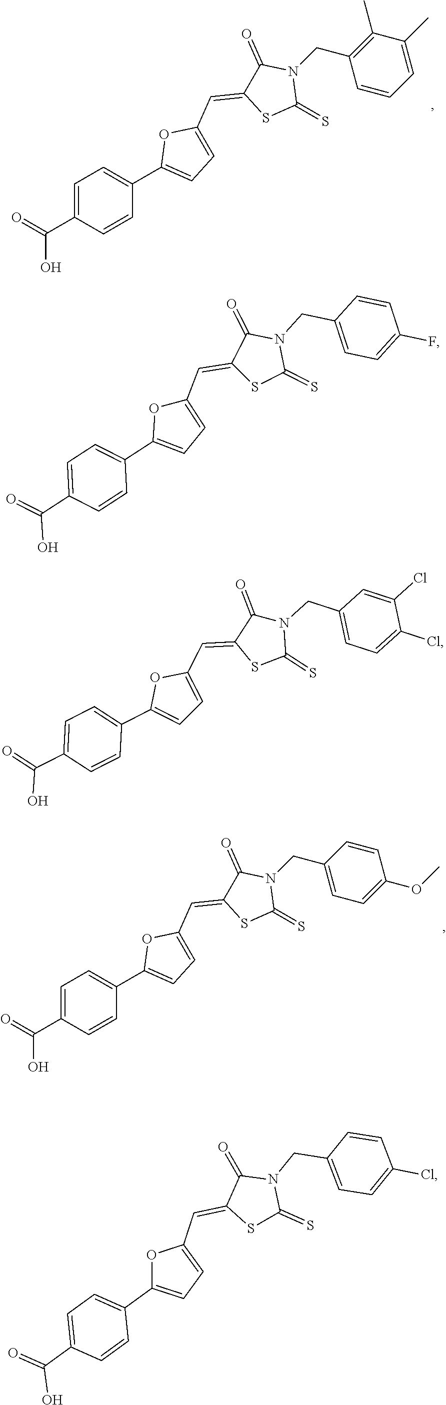 [SMILES: Cc1cccc(CN2C(=O)/C(=C/c3ccc(-c4ccc(C(=O)O)cc4)o3)SC2=S)c1C ; O=C(O)c1ccc(-c2ccc(/C=C3\SC(=S)N(Cc4ccc(F)cc4)C3=O)o2)cc1 ; O=C(O)c1ccc(-c2ccc(/C=C3\SC(=S)N(Cc4ccc(Cl)c(Cl)c4)C3=O)o2)cc1 ; COc1ccc(CN2C(=O)/C(=C/c3ccc(-c4ccc(C(=O)O)cc4)o3)SC2=S)cc1 ; O=C(O)c1ccc(-c2ccc(/C=C3\SC(=S)N(Cc4ccc(Cl)cc4)C3=O)o2)cc1]

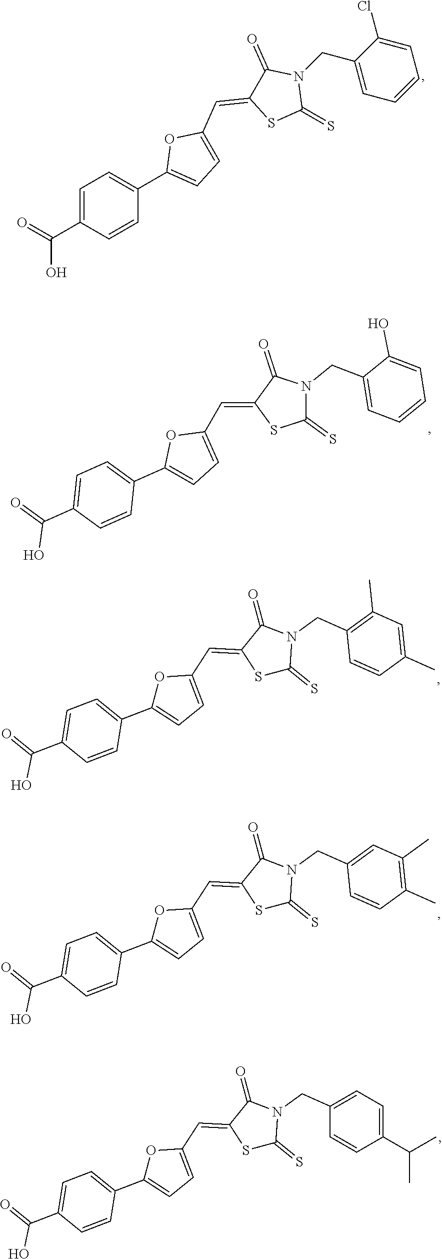 [SMILES: O=C(O)c1ccc(-c2ccc(/C=C3\SC(=S)N(Cc4ccccc4Cl)C3=O)o2)cc1 ; O=C(O)c1ccc(-c2ccc(/C=C3\SC(=S)N(Cc4ccccc4O)C3=O)o2)cc1 ; Cc1ccc(CN2C(=O)/C(=C/c3ccc(-c4ccc(C(=O)O)cc4)o3)SC2=S)c(C)c1 ; Cc1ccc(CN2C(=O)/C(=C/c3ccc(-c4ccc(C(=O)O)cc4)o3)SC2=S)cc1C ; CC(C)c1ccc(CN2C(=O)/C(=C/c3ccc(-c4ccc(C(=O)O)cc4)o3)SC2=S)cc1]

and pharmaceutically acceptable salts thereof.

In some embodiments, the compound has the structure:

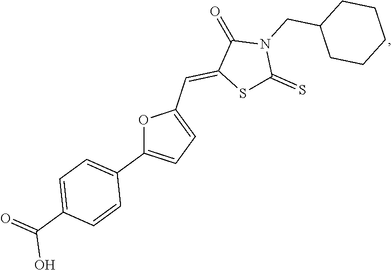 [SMILES: O=C(O)c1ccc(-c2ccc(/C=C3\SC(=S)N(CC4CCCCC4)C3=O)o2)cc1]

and pharmaceutically acceptable salts thereof.

In some embodiments, the invention provides a compound having a structure according to Formula XIVd:

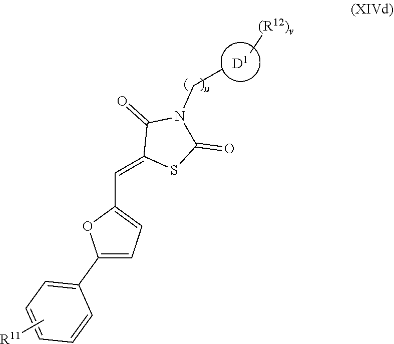

or a pharmaceutically acceptable salt thereof, wherein
R¹¹ is COOR^{11a};
R^{11a} is selected from H and C₁₋₆ alkyl; and
each R¹² is independently selected from halogen, C₁₋₆ alkyl, and C₁₋₆ alkoxy.

In some embodiments, R^{11a} is H in compounds of Formula XIVd. In some embodiments, R^{11a} is C₁₋₆ alkyl in compounds of Formula XIVd. In some such embodiments, R^{11a} is selected from methyl, ethyl, n-propyl, isopropyl, n-butyl, sec-butyl, and tent-butyl. In some embodiments, R^{11a} is selected from COOH and COOMe.

In some embodiments, the invention provides compounds of Formula XIVd, wherein subscript u is 0 and subscript v is 0, 1, 2, or 3. In some embodiments, the invention provides compounds of Formula XIVd, wherein subscript u is 1 and subscript v is 0, 1, 2, or 3. In some embodiments, the invention provides compounds of Formula XIVd, wherein subscript u is 2 and subscript v is 0, 1, 2, or 3. In some embodiments, the invention provides compounds of Formula XIVd, wherein subscript u is 0, 1, or 2 and subscript v is 0. In some embodiments, the invention provides compounds of Formula XIVd, wherein subscript u is 0, 1, or 2 and subscript v is 1. In some embodiments, the invention provides compounds of Formula XIVd, wherein subscript u is 0, 1, or 2 and subscript v is 2. In some embodiments, the invention provides compounds of Formula XIVd, wherein subscript u is 0, 1, or 2 and subscript v is 3.

### III. PHARMACEUTICAL COMPOSITIONS

In a related aspect, the invention provides pharmaceutical compositions for the administration of the integrin agonists described herein. The pharmaceutical compositions can be prepared by any of the methods well known in the art of pharmacy and drug delivery. In general, methods of preparing the compositions include the step of bringing the active ingredient into association with a carrier containing one or more accessory ingredients. The pharmaceutical compositions are typically prepared by uniformly and intimately bringing the active ingredient into association with a liquid carrier or a finely divided solid carrier or both, and then, if necessary, shaping the product into the desired formulation. The compositions can be conveniently prepared and/or packaged in unit dosage form.

The pharmaceutical compositions can be in the form of sterile injectable aqueous or oleaginous solutions and suspensions. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Aqueous suspensions contain the active materials in admixture with excipients including, but not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate.

Oily suspensions can be formulated by suspending the active ingredient in a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol. These compositions can be preserved by the addition of an anti-oxidant such as ascorbic acid.

Dispersible powders and granules (suitable for preparation of an aqueous suspension by the addition of water) can contain the active ingredient in admixture with a dispersing agent, wetting agent, suspending agent, or combinations thereof. Additional excipients can also be present.

The pharmaceutical compositions of the invention can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

Pharmaceutical compositions containing the integrin agonists described herein can also be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredient is mixed with water or an oil medium (such as peanut oil, liquid paraffin, or olive oil).

The integrin agonists described herein can also be administered topically as a solution, ointment, cream, gel, suspension, mouth washes, eye-drops, and the like. Still further, transdermal delivery of the integrin agonists can be accomplished by means of iontophoretic patches and the like. The compound can also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

In some embodiments, an integrin agonist described herein is administered via intraperitoneal injection. In some embodiments, the integrin agonist is administered orally. In some embodiments, the integrin agonist is administered intravenously.

The integrin agonists described herein can be used in combination with drugs selected from, but not limited to, 5-fluorouracil, AZD8055, bevacizumab, bortezomib, cetuximab, cyclophosphamide, docetaxel, gemcitabine, imatinib, ipilimumab, lapatinib, paclitaxel, pembrolizumab, pertuzumab, rapamycin, sipuleucel-T, sorafenib, sunitinib, trastuzumab, temsirolimus, vemurafenib, taxol, paclitaxel, abiraterone, steroids, corticosteroids, prednisone, NSAIDs, mitomycin, androgens, antiandrogens, estrogens, antiestrogens, statins, CTLA-4 inhibitors, anti-CTLA-4 antibodies, B7 modulators, abatacept, rituximab, belatacept, benlumimab, PD-1 modulators, anti-PD1 antibodies, PDL1 modulators, anti-PDL1 antibodies, IDO1 inhibitors and modulators, CSF1 modulators, CSF1R modulators, anti-CSF1R antibodies, PI3K inhibitors, MEK inhibitors, JAK inhibitors, STAT inhibitors, CD40 modulators, CD47 modulators and inhibitors, CD206 modulators and inhibitors, TNFα inhibitors and modulators, anti-TNFα antibodies, cytokine modulators, anti-cytokine antibodies, interleukin modulators and inhibitors, anti-interleukin antibodies, anti-CCL2, anti-CCL4, CXCR-4 inhibitors, anti-CXCR4, IL10 and IL10 analogs, anti-IL10, anti-IL17, and anti-IL23.

The pharmaceutical compositions of the invention can also include micronized integrin agonist. Micronized integrin agonist particles generally consist essentially of the integrin agonist with average diameters below 50 μm. The average diameter of the integrin agonist particles can be, for example, below 45 μm, below 40 μm, below 35 μm, below 30 μm, below 25 μm, or below 20 μm. The average diameter of the integrin agonist particles can be from about 10 μm to about 49 μm, or from about 10 μm to about 45 μm, or from about 15 μm to about 40 μm, or from about 20 μm to about 35 μm, or from about 25 μm to about 30 μm. The average diameter of the integrin agonist particles can be about 15, 16, 17, 18, 19, 20, 21, 22, 23, 24, or about 25 μm.

### IV. METHODS OF TREATING INTEGRIN-MEDIATED DISEASES AND CONDITIONS

The integrin agonists described herein can be used for treating a disease or condition associated with the activity of β2 integrins. In some embodiments the β2 integrin is CD11b/CD18. In some embodiments the β2 integrin is CD11a/CD18. In some embodiments the β2 integrin is CD11c/CD18. In some embodiments the β2 integrin is CD11d/CD18. In certain embodiments, such a disease or condition is selected from aortic aneurisms; arteritis; asthma; atherosclerosis; autoimmune diseases (including lupus, psoriasis, Crohn's disease, multiple sclerosis, bullous pemphigoid, and rheumatoid arthritis); bowel conditions (including irritable bowel syndrome, inflammatory bowel disease, and ulcerative colitis); burn; cachexia including cardiac cachexia; cancer, including primary tumors and metastatic tumors, such as breast cancer, ovarian cancer, prostate cancer, melanoma, lung cancer, pancreatic cancer, sarcoma, tenosynovial giant cell tumor and leukemia; cardiovascular disease; Chediak-Higashi syndrome; chronic granulomatous disease; chronic kidney disease; complications of coronary by-pass surgery; diabetes; diabetic nephropathy; dyslipidemia; encephalomyelitis; familial hypercholesterolemia; fibrosis; focal segmental glomerulosclerosis; glomerulonephritis; giant cell arteritis; glaucoma; graft-versus-host disease; heart failure including chronic heart failure (CHF); hyper-IgM syndromes; hypertension; immune deficiency (including but not limited to acquired immune deficiency syndrome (AIDS), HIV infection, and severe combined immunodeficiency); complications of long-term anti-viral treatment, including anti-HIV treatment; inflammation (including but not limited to acute and chronic inflammation); inflammatory skin diseases (including but not limited to psoriasis and dermatitis); iron deficiency; ischemia-reperfusion injury (including but not limited to ischemia-reperfusion injury following myocardial infarction and post-ischemic cerebral inflammation); leukocyte adhesion deficiency; macular degeneration; myeloperoxidase deficiency; myocardial infarction; myocarditis including chronic autoimmune myocarditis and viral myocarditis; neointimal thickening associated with vascular injury; neonatal obstructive nephropathy; neurological conditions (including Alzheimer's disease, Parkinson's disease, and traumatic brain injury); obesity; pain (including chronic pain and osteoarthritic pain); post-implantation complications of left ventricular assist devices; radiation injury; restenosis after heart surgery; sepsis and septic shock; scarring; silent myocardial ischemia; stenosis; stroke; thrombophlebitis; transplant rejection; uveitis; vascular occlusion including cerebral artery occlusion; vasculitis including Kawasaki's vasculitis; Wegener's granulomatosis; Wiskott-Aldrich syndrome; and wound healing.

In some embodiments, the integrin agonists can be used in conjunction with processes such as transplantation (including cell transplantation, organ transplantation, and bone marrow transplantation); organ and cell preservation; and stem cell therapies.

In some embodiments, the disease or condition associated with the activity of β2 integrins is kidney disease, a condition that affects millions of people in the world and leads to renal failure. In some embodiments, the disease or condition associated with the activity of β2 integrins is restenosis. Restenosis is a frequent problem in people who have undergone angioplasty, one of the most common procedures in interventional cardiology.

The integrin agonists described herein can be used for treating cancer or reducing tumors in patients. In certain embodiments, the integrin agonists modulate tumor infiltration of leukocytes. Tumors secrete inflammatory cytokines to recruit cells expressing β2 integrins, such as CD11b/CD18, to facilitate neovascularization. During cancer treatments, including via chemotherapy and irradiation, tumors recruit large numbers of specific leukocytes or bone marrow-derived cells that restore tumor vasculature and allow tumor re-growth and recurrence. Therefore, the compounds and methods of this invention are useful in reducing activity, such as infiltration, of such cells. In addition, activating CD11b can enhance anti-tumor immune responses. Similarly, activating integrins can modify polarization of microglia and myeloid cells, such as macrophages, thus modifying the tumor and tissue microenvironment. Accordingly, compounds that agonize CD11b, including the integrin agonists described herein as well as other compounds, can be used to target and exploit immunomodulatory pathways for anti-tumor therapy. In some embodiments, the integrin agonists described herein are useful in enhancing the response of other cancer treatments, such as chemotherapy, antibody therapy, radiation therapy, and cell-based therapies.

In some embodiments, the integrin agonists forms described can be used to decrease leukocyte recruitment upon injury, inflammation, bacterial infection, viral infection, or other diseases and conditions in mammals. In some embodiments, the integrin agonists can be used to reduce organ injury, including neointimal hyperplasia upon arterial injury. In some embodiments, the integrin agonists can be used to preserve organ function upon acute organ injury, such as ischemia-reperfusion injury. For example, the integrin agonists can preserve kidney function upon acute kidney injury. In some embodiments, the integrin agonists described herein can be used to preserve kidney function upon glomerular nephritis or nephrosis.

In some embodiments, the integrin agonists described herein can be used to modulate the function of inflammatory cells, such as lymphocytes and leukocytes. The compounds can be used to treat integrin-mediated inflammation in a number of organs and tissues including, but not limited to, integrin-mediated inflammation of the eye, the brain, the skin, the liver, and the kidney. For example, the integrin agonists can be used to induce graft tolerance in a recipient animal. Grafts can include bone marrow, bone marrow cells, stem cells, immune cells, engineered cells, organs, tissues or other cells. Similarly, the integrin agonists can reduce graft-vs-host disease in the recipient. Thus, the integrin agonists can improve transplantation outcomes.

Accordingly, the invention provides methods for preventing or treating a β2 integrin-mediated condition or disease in a patient comprising administering to said patient a therapeutically effective amount of an integrin agonist described herein. In certain embodiments, the β2 integrin-mediated condition or disease is a CD11b/CD18-mediated condition or disease.

The integrin agonists described herein can be used to treat conditions related to the activity of adenosine A2A receptors and glucocorticoid receptors.

In some embodiments, the integrin agonists are used for the treatment of neurological conditions such as Alzheimer's disease. In some embodiments, treatment of the neurological condition includes modulation of microglia cell function by the integrin agonist. In certain embodiments, treatment of Alzheimer's disease includes reduction of soluble Aβ levels and Aβ half-life in brain interstitial fluid (ISF) by the integrin agonist. Treatment of Alzheimer's disease can include co-administration of an integrin agonist as described herein with an Aβ-targeted therapy, an ApoE-targeted therapy, a tau-targeted therapy, or a combination thereof. Aβ-targeted therapies include inhibitors of Aβ production (such as beta-secretase inhibitors, gamma-secretase inhibitors, alpha-secretase activators), inhibitors of Aβ aggregation, inhibitors of Aβ oligomerization, and up-regulators of Aβ clearance, among others (see, e.g., Jia, et al. BioMed Research International, 2014. Article ID 837157, 22 pages). Examples of Aβ-targeted therapies include but are not limited to, antibodies, pioglitazone, begacestat, atorvastatin, simvastatin, etazolate, and tramiprosate, as well as pharmaceutically acceptable salts thereof. Examples of ApoE-targeted therapies include, but are not limited to retinoid X receptor agonists (see, Cramer, et al., Science 2012. 335(6075): 1503-1506) and others described by Liu et al. (Nat Rev Neurol. 2013. 9(2): 106-118). Tau-targeted therapies include, but are not limited to, methylthioninium, leuco-methylthioninium, antibodies and those described by Lee, et al. (Cold Spring Harb Perspect Med 2011; 1:a006437).

In some embodiments, the integrin agonists are used for the treatment of inflammatory bowel diseases such as Crohn's disease or ulcerative colitis. In certain embodiments, treatment of Crohn's disease and/or ulcerative colitis can include co-administration of an integrin agonist as described herein with an anti-inflammatory. For example, the anti-inflammatory can be an NSAID (e.g., apazone, diclofenac, ibuprofen, indomethacin, ketoprofen, nabumetone, naproxen, piroxicam, and sulindac, as well as pharmaceutically acceptable salts thereof), a TNFα inhibitor/modulator (e.g., an anti-TNFα antibody such as adalimumab), an anti-interleukin agent (e.g., an anti-IL6 receptor antibody such tocilizumab or an anti-IL5 antibody such as siltuximab), or an anti-inflammatory cytokine (e.g., recombinant IL-4, IL-10, or IL-13).

In another embodiment, the invention provides a method for treating cancer. The method comprises administering to a subject in need thereof a therapeutically effective amount of an integrin agonist as described herein, or a pharmaceutically acceptable salt thereof, and a therapeutically effective amount of an immune checkpoint inhibitor.

“Immune checkpoint” refers to a regulatory pathway that contributes to co-stimulatory or inhibitory control of T-cell activity in an organism. Interaction of “immune checkpoint proteins,” including proteins on the surfaces of antigen-presenting cells and T-cells, contribute to regulation and maintenance of self-tolerance and the duration and amplitude of physiological immune responses in the organism. See, e.g., D. M. Pardol. Nature Reviews Cancer 12, 252-264 (2012). Examples of immune checkpoint proteins include, but are not limited to, A2aR (adenosine A2a receptor); BTLA, B, and T (lymphocyte attenuator); ICOS (inducible T cell co-stimulator); KIR (killer cell immunoglobulinlike receptor); LAG3 (lymphocyte activation gene 3); PD1 (programmed cell death protein 1); CTLA-4 (cytotoxic T-lymphocyte-associated antigen 4); and TIM3 (T cell membrane protein 3).

“Immune checkpoint inhibitor” refers to a molecule that totally or partially reduces, inhibits, interfere with, or otherwise modulates the activity of one or more checkpoint proteins. Immune checkpoint inhibitors can, for example, include antibodies or peptide-like compounds derived from antibodies.

In some embodiments, the method comprises administering to the subject a pharmaceutically acceptable salt of the compound according to Formula I. In some embodiments, the salt is a meglumine salt or a choline salt. In some such embodiments, the invention includes administering an integrin agonist as described herein.

In some embodiments, the immune checkpoint inhibitor inhibits the activity of one or more targets selected from the group consisting of CTLA-4, 4-1BB (CD137), 4-1BBL (CD137L), PDL1, PDL2, PD1, B7-H3, B7-H4, BTLA, HVEM, TIM3, GALS, LAG3, TIM3, B7H3, B7H4, VISTA, KIR, 2B4, CD160, IDO1/IDO2 (indoleamine 2,3-dioxygenase), and CGEN-15049.

In some embodiments, the immune checkpoint inhibitor is a protein that binds to one or more targets selected from the group consisting of CTLA-4, PDL1, PDL2, PD1, B7-H3, B7-H4, BTLA, HVEM, TIM3, GALS, LAG3, TIM3, B7H3, B7H4, VISTA, KIR, 2B4, CD160, and CGEN-15049.

“PD1” refers to programmed cell death protein 1, also known as CD279, expressed by T-cells, B-cells, and monocytes. PD-1 is a type I surface glycoprotein characterized by a V-set immunoglobulin superfamily (IgSF) domain attached to a transmembrane domain and a cytoplasmic domain containing two tyrosine-based signaling motifs. PD1 binds at least two ligands: PD-L1 (expressed by cells including T-cells, B-cells, dendritic cells, macrophages, and mesenchymal stem cells) and PD-L2 (expressed by cells including dendritic cells, macrophages, and mast cells).

“CTLA-4” refers to cytotoxic T-lymphocyte-associated antigen 4, also known as CD152, which is expressed exclusively on T-cells. CTLA-4 includes a single Ig-fold extracellular domain with three CDR-like loops, and binds to ligands CD80 (B7.1) and CD86 (B7.2), among others, that are differentially expressed in antigen presenting cells.

In some embodiments, the immune checkpoint inhibitor is selected from the group consisting of an antibody and an antigen-binding antibody fragment. In some embodiments, the immune checkpoint inhibitor is selected from the group consisting of a CTLA-4 antibody, an OX40 antibody, a PD-L1 antibody, a PD1 antibody, and a BY55 antibody. In some embodiments, the immune checkpoint inhibitor is a CTLA-4 antibody. In some embodiments, the immune checkpoint inhibitor is a PD1 antibody.

In some embodiments, the immune checkpoint inhibitor is selected from the group consisting of tremelimumab, MEDI4736, MK-3475, nivolumab, CT-011, AMP224, BMS-936559, MPLDL3280A, MSB0010718C, and ipilimumab.

In some embodiments, the cancer is associated with expression of one or more leukocyte markers in the subject.

“Leukocyte marker” refers to a biomolecule (e.g., a polypeptide) found on the cell surface of a leukocyte. Leukocyte markers include, but are not limited to, T-cell antigen receptors; CD1; NK cell receptors; IDO1/2; TDO; CSF1R; VEGFR; SIRPa; cell adhesion molecules (e.g., CD2, CD58 (LFA-3), CD3, CD4, CD5, CD7, CD8); β2 integrins (e.g., LeuCAM, CD11a (LFA-1), CD11b (MAC-1 (CR3)), CD11c (CR4), CD11d, CD18, CD16 (FcR111), CD21 (CR2), CD23, CD25, CD30, CD35 (CR1)); β3 integrins (e.g., CD41, CDS1); homing receptors (e.g., CD44, Mel-14); β1 integrins (e.g., CD49a-f (VLA-1), VLA-2, VLA-3, VLA-4); CD14; CD56; CD68; CD71; and CD163.

In some embodiments, the leukocyte markers are selected from the group consisting of CD11b/CD18, IDO1/2, TDO, CSF1R, CD14, CD16, CD68, VEGFR, and SIRPa.

In some embodiments, the cancer expresses one or more targets for β2 integrins. In some embodiments, the targets are selected from the group consisting of ICAM-1, VCAM-1, fibronectin, vironectin, fibrinogen, and complement fragments.

In some embodiments, the cancer is selected from the group consisting of a melanoma, a sarcoma, a lymphoma, a glioma, a leukemia, pancreatic cancer, a tenosynovial giant-cell tumor, breast cancer, renal cancer, ovarian cancer, prostate cancer, colon cancer, stomach cancer, and lung cancer. In some embodiments, the cancer is a melanoma. In some embodiments, the cancer patient has also been diagnosed with an autoimmune disease (e.g., multiple sclerosis, lupus, rheumatoid arthritis, Crohn's disease, or ulcerative colitis).

In another aspect, the invention provides a method for treating melanoma. The method comprises administering to a subject in need thereof an integrin agonist as described herein, or a pharmaceutically acceptable salt thereof, and a therapeutically effective amount of a PD1 antibody.

In another aspect, the invention provides a method for treating cancer which includes administering to a subject in need thereof an integrin agonist as described herein, or a pharmaceutically acceptable salt thereof, and a therapeutically effective amount of an agent that targets myeloid cells.

“Myeloid cell” generally refers to any white blood cell (i.e., leukocyte) which is not a lymphocyte (e.g., not a natural killer cell, T cell, or B cell). Myeloid cells include macrophages, dendritic cells, and granulocytic cells.

In some embodiments, the agent that targets myeloid cells inhibits the activity of one or more targets selected from the group consisting of CSF1R, IDO1/2, TDO, CCR2, CCL2, CXCR4, JAK1/2/3/4/5, PI3Kg, integrin β1, integrin α4β1 (VLA4), VEGFR.

In some embodiments, the agent that targets myeloid cells increases the activity of SIRPa.

In some embodiments of any one of the preceding aspects, the method further comprises detecting one or more leukocyte markers in a sample obtained from the subject, thereby identifying the subject as needing the treatment. In some such embodiments, the leukocyte markers are selected from the group consisting of CD11b/CD18, CD11b, CD18, CD11c, CD11d, IDO1/2, TDO, CSF1R, CD14, CD16, CD68, VEGFR, and SIRPa. In some such embodiments, the marker is CD11b/CD18.

In some embodiments of any one of the preceding aspects, the method further comprises monitoring treatment efficacy by imaging tumor cells with macrophage-targeted imaging agents. In some embodiments of any on the preceding aspects, the method further comprises monitoring treatment efficacy by monitoring levels of one or more macrophage markers in the subject.

In a related aspect, the invention provides a method for reducing CD11b+ leukocytes in a tumor. The method comprises administering to a subject in need thereof an integrin agonist as described herein, or a pharmaceutically acceptable salt thereof, and an effective amount of an agent selected from the group consisting of an immune checkpoint inhibitor, an agent that targets myeloid cells, and combinations thereof

In some embodiments, the CD11b+ leukocytes are myeloid cells. In some embodiments, the CD11b+ leukocytes are macrophages. In some embodiments, the CD11b+ leukocytes are neutrophils.

In some embodiments, the ratio of anti-tumorigenic to pro-tumorigenic macrophages in the tumor tissue is changed.

In some embodiments, the M1/M2 macrophage ratio or M1-like/M2-like ratio is changed in the tumor. In some such embodiments, the macrophages are polarized more toward an M1 phenotype or an M1-like anti-tumor macrophage phenotype after treatment.

In some embodiments, the invention provides a method for preventing tumor metastasis in a subject having cancer. The method includes administering an integrin agonist as described herein, or a pharmaceutically acceptable salt thereof, and reducing infiltration of CD11b+ leukocytes in a potential metastasis site in the subject.

In some embodiments, the method for preventing tumor metastasis further includes administering an effective amount of an agent selected from the group consisting of an immune checkpoint inhibitor, an agent that targets myeloid cells, and combinations thereof.

The integrin agonists described herein can be administered at any suitable dose in the methods of the invention. In general, an integrin agonist is administered at a dose ranging from about 0.1 milligrams to about 2000 milligrams per kilogram of a subject's body weight (i.e., about 0.1-2000 mg/kg). The dose of the integrin agonist can be, for example, about 0.1-1000 mg/kg, or about 1-500 mg/kg, or about 25-250 mg/kg, or about 50-100 mg/kg, or about 10-100 mg/kg. The dose of the integrin agonist can be about 1, 2, 3, 4, 5, 10, 15, 20, 25, 30, 35, 40, 45, 50, 55, 60, 65, 70, 75, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 mg/kg. The dose of the integrin agonist can be administered at a dose below about 1, below about 2, below about 3, below about 4, below about 5, below about 10, below about 15, below about 20, below about 25, below about 30, below about 35, below about 40, below about 45, below about 50, below about 55, below about 60, below about 65, below about 70, below about 75, below about 85, below about 90, below about 95, below about 100, below about 150, below about 200, below about 250, below about 300, below about 350, below about 400, below about 450, below about 500, below about 550, below about 600, below about 650, below about 700, below about 750, below about 800, below about 850, below about 900, below about 950, or below about 1000 mg/kg. In some embodiments, the integrin agonist is administered at a dose below 200 mg of compound per kg of the subject's body weight (200 mg/kg). In some embodiments, the integrin agonist is administered at a dose below 100 mg/kg. In some embodiments, the integrin agonist is administered at a dose below 50 mg/kg. In some embodiments, the integrin agonist is administered at a dose below 20 mg/kg.

Immune checkpoint inhibitors can be administered at any suitable dose in the methods of the invention. In certain embodiments, an antibody immune checkpoint inhibitor is administered at a dose ranging from about 0.1 milligrams to about 100 milligrams per kilogram of a subject's body weight (i.e., about 0.1-100 mg/kg). The dose of the antibody immune checkpoint inhibitor can be, for example, about 0.1-50 mg/kg, or about 1-10 mg/kg. The dose of the antibody immune checkpoint inhibitor can be about 1, 2, 3, 4, 5, 6, 7, 8, 9, 10, 11, 12, 13, 14, 15, 16, 17, 18, 19, or 20 mg/kg.

The dosages can be varied depending upon the requirements of the patient, the severity of the β2 integrin-mediated disorder or condition being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the integrin-mediated condition.

Administration of an integrin agonist described herein can be conducted for a period of time which will vary depending upon the nature of the particular the β2 integrin-mediated disorder or condition, its severity and the overall condition of the patient. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a patient can be monitored for changes in his or her condition and for alleviation of the symptoms of the β2 integrin-mediated disorder or condition. The dosage of the integrin agonist can either be increased in the event the patient does not respond significantly to a particular dosage level, or the dose can be decreased if an alleviation of the symptoms of the β2 integrin-mediated disorder or condition is observed, or if the disorder or condition has been ablated, or if unacceptable side effects are seen with a particular dosage.

A therapeutically effective amount of an integrin agonist described herein can be administered to the subject in a treatment regimen comprising intervals of at least 1 hour, or 6 hours, or 12 hours, or 24 hours, or 36 hours, or 48 hours between dosages. Administration can be conducted at intervals of at least 72, 96, 120, 168, 192, 216, or 240 hours, or the equivalent amount of days. The dosage regimen can consist of two or more different interval sets. For example, a first part of the dosage regimen can be administered to a subject multiple times daily, daily, every other day, or every third day. The dosing regimen can start with dosing the subject every other day, every third day, weekly, biweekly, or monthly. The first part of the dosing regimen can be administered, for example, for up to 30 days, such as 7, 14, 21, or 30 days. A subsequent second part of the dosing regimen with a different interval administration administered weekly, every 14 days, or monthly can optionally follow, continuing for 4 weeks up to two years or longer, such as 4, 6, 8, 12, 16, 26, 32, 40, 52, 63, 68, 78, or 104 weeks. Alternatively, if the β2 integrin-mediated disorder or condition goes into remission or generally improves, the dosage may be maintained or kept at lower than maximum amount. If the disorder or condition relapses, the first dosage regimen can be resumed until an improvement is seen, and the second dosing regimen can be implemented again. This cycle can be repeated multiple times as necessary.

In certain embodiments, an integrin agonist and an immune checkpoint inhibitor are administered in synergistic amounts; in such cases the effect of the agents when administered in combination is greater than the additive effect of the compounds when administered alone as a single agent. In some embodiments, the synergistic effect is obtained by administering the integrin agonist and the checkpoint inhibitor at concentrations below the maximally effective concentration of the drugs when administered as single agents. The synergistic amounts can depend on factors including, but not limited to, the particular integrin agonist, the particular immune checkpoint inhibitor, the condition (e.g., cancer type) being treated, and the route and frequency of administration. Synergy can be observed in terms of lower cytotoxicity, increased anti-proliferative and/or anti-infective effect, or some other beneficial effect of the combination compared with the individual components.

In some embodiments, an integrin agonist as described above is administered to the subject in an amount ranging from about 1 mg/kg to about 2000 mg/kg. In some such embodiments, the immune checkpoint inhibitor is administered in a synergistic amount with the integrin agonist. In some of these embodiments, the integrin agonist is administered orally to the subject.

In some embodiments, an integrin agonist is administered to the subject in an amount ranging from about 2 mg/kg to about 100 mg/kg. In some such embodiments, the immune checkpoint inhibitor is administered in a synergistic amount with the integrin agonist. In some of these embodiments, the integrin agonist is administered orally to the subject.

The integrin agonists described herein can modulate the release of one or more secreted factors, including but not limited to cytokines and chemokines, from leukocytes. Cytokines include pro-inflammatory cytokines (e.g., interleukin (IL)-1, tumor necrosis factor (TNF)) and anti-inflammatory cytokines (e.g., IL-4, IL-10, IL-13). In certain embodiments, administration of an integrin agonist described herein results in modulation of cytokine expression (or other soluble factor) by the integrin agonist. In some embodiments, the cytokine is selected from IL-1β, IL-6, and IL-10. In some embodiments, the soluble factor is selected from TNF-α, interferon a (IFNa), interferon b (IFNb) and interferon (IFN)-γ. Soluble factors such as cytokines are inflammatory markers and can be assayed in patient sera or patient-derived cells or tissues to assess the efficacy of a particular integrin agonist in treating a particular condition. A number of diagnostic assays for cytokines such IL-1β and TNF-α are known in the art and can be used to assess the anti-inflammatory efficacy of an integrin agonist. Such methods include, but are not limited to, ELISA (enzyme-linked immune-sorbent assay) and bead array systems for capture of cytokines by resin-bound antibodies and detection by flow cytometry.

In another aspect, the invention provides a method for treating cancer, wherein the method includes: determining the expression level of one or more proteins selected from the group consisting of CD11b, CD18, IDO1, IDO2, TDO, CSF1R, CD14, CD16, CD68, VEGFR, SIRPa, ARG1, UPAR, CD114, CD11a, CD11c, CD11d, CD40, A2Ra, CD47, CD45, CD4, CD8, FOXP3, CD3, ICAM1, CD31, DESMIN, alpha-smooth muscle actin, TGFβ, matrix metalloproteinases, CD64, CD32, and CD89 in the subject, and administering a therapeutically effective amount of an integrin agonist as described herein to the subject. In some embodiments, determining the expression level of the proteins includes obtaining a biospecimen (such as a biopsy) from the patient and determining the expression level of the proteins in the biospecimen. In some such embodiments, the method further includes administering a therapeutically effective amount of an immune checkpoint inhibitor to the subject. In some such embodiments, the method further comprises periodically determining the expression level of the protein over the course of an evaluation period, and adjusting the treatment if the expression level of the protein is observed to change over the course of the evaluation period.

In some embodiments, the method includes determining that the expression level of a protein in a biospecimen, such as a biopsy, obtained from a subject is higher than the expression level of the protein in a biospecimen sample obtained from a healthy subject. In some embodiments, the method includes determining that the expression level of a protein in a biopsy sample obtained from a subject is higher than the expression level of the protein in a non-cancerous tissue sample obtained from the subject. In some such embodiments, the expression level of one more proteins selected from the group consisting of CD11b, CD18, IDO1, IDO2, TDO, CSF1R, CD14, CD16, CD68, VEGFR, SIRPa, ARG1, UPAR, CD114, CD11a, CD11c, CD11d, CD45, CD4, CD8, FOXP3, CD3, ICAM1, CD31, DESMIN, alpha-smooth muscle actin, CD64, CD32, and CD89 is determined.

In some embodiments, the method includes determining that the expression level of a protein in a biospecimen, such as a biopsy, obtained from a subject is lower than the expression level of the protein in a biospecimen sample obtained from a healthy subject. In some embodiments, the method includes determining that the expression level of a protein in a biopsy sample obtained from a subject is lower than the expression level of the protein in a non-cancerous tissue sample obtained from the subject. In some such embodiments, the expression level of one more proteins selected from the group consisting of CD11b, CD18, IDO1, IDO2, TDO, CSF1R, CD14, CD16, CD68, VEGFR, SIRPa, ARG1, UPAR, CD114, CD11a, CD11c, CD11d, CD45, CD4, CD8, FOXP3, CD3, ICAM1, CD31, DESMIN, alpha-smooth muscle actin, CD64, CD32, and CD89 is determined.

In some embodiments, the invention provides a method for treating cancer, wherein the method includes: determining the level of one or more substances selected from the group consisting of colony stimulating factor 1 (CSF1); C-reactive protein (CRP); urokinase receptor (uPAR); soluble urokinase-type plasminogen activator receptor (suPAR); Glypican-1; CD11b; vascular endothelial growth factor (VEGF); VEGF receptor; a matrix metalloproteinase such as MMP-9 and the like; TNFα; an interleukin such as IL-6, IL-1β, IL-10, IL-17, IL-23, and the like; TGFβ; interferons including IFN-α, IFN-β, and the like; tryptophan; lysine; arginine; lactate; and a microRNA in the subject, and administering a therapeutically effective amount of integrin agonist as described herein to the subject having the biomarker. In some embodiments, determining the level of the substance includes obtaining a blood, plasma, urine, or saliva sample from the patient and determining the expression level of the proteins in the sample. In some such embodiments, the method further includes administering a therapeutically effective amount of an immune checkpoint inhibitor to the subject. In some such embodiments, the method further comprises periodically determining the level of the substance over the course of an evaluation period, and adjusting the treatment if the level of the substance is observed to change over the course of the evaluation period.

In some embodiments, the method includes determining that the level of the substance in a blood, plasma, urine, or saliva sample obtained from a subject is higher than the expression level of the protein in a similar plasma sample obtained from a healthy subject. In some embodiments, the method includes determining that the level of the substance in a blood, plasma, urine, or saliva sample obtained from a subject is lower than the level of the substance in a similar sample obtained from a healthy subject. In some such embodiments, the level of one more substances selected from the group consisting of colony stimulating factor 1 (CSF1); C-reactive protein (CRP); urokinase receptor (uPAR); soluble urokinase-type plasminogen activator receptor (suPAR); Glypican-1; CD11b; vascular endothelial growth factor (VEGF); VEGF receptor; a matrix metalloproteinase such as MMP-9 and the like; TNFα; an interleukin such as IL-6, IL-1β, IL-10, IL-17, IL-23, and the like; TGFβ; interferons including IFN-α, IFN-β, and the like; tryptophan; lysine; arginine; lactate; and a microRNA is determined.

### V. EXAMPLES

### Example 1. Preparation of (Z)-4-(5-((3-Benzyl-4-oxo-2-thioxooxazolidin-5-ylidene)methyl)furan-2-yl)benzoic acid (1)

The title compound was prepared according to Scheme 1.

Synthesis of 3-benzyl-2-thioxooxazolidin-4-one (1.1). To a solution of methyl 2-hydroxyacetate (1.2a, 2.0 g, 13.42 mmol) in dichloromethane (20 mL), triethylamine (3.76 mL, 26.8 mmol) was added in one portion followed by addition of (isothiocyanatomethyl)benzene (1.2, 1.20 g, 13.42 mmol) at 0° C. The reaction mixture was stirred at room temperature for 48 h. After completion, water (50 mL) was added to the reaction and extracted with ethyl acetate (100 mL). The organic layer was dried over anhydrous sodium sulfate and concentrated under reduced pressure to get crude. The crude product was purified by silica gel (100-200 mesh) column chromatography eluting with 5-10% ethyl acetate in hexanes to afford 3-benzyl-2-thioxooxazolidin-4-one (1.1) as yellowish solid. Yield: 1.10 g, 39.7%.

Synthesis of (Z)-4-(5-((3-benzyl-4-oxo-2-thioxooxazolidin-5-ylidene)methyl)furan-2-yl)benzoic acid (1). To a solution of 3-benzyl-2-thioxooxazolidin-4-one (1.1, 0.250 g, 1.20 mmol) in acetic acid (10 mL), sodium acetate (0.148 g, 1.81 mmol) and 4-(5-formylfuran-2-yl)benzoic acid (1.1a, 0.235 g, 1.08 mmol) were added at room temperature. The reaction mixture was heated at 120° C. for 18 h. After completion, water (20 mL) was added and the precipitate formed was collected by filtration. The product was purified by washings with water, diethyl ether and n-pentane to afford (Z)-4-(5-((3-benzyl-4-

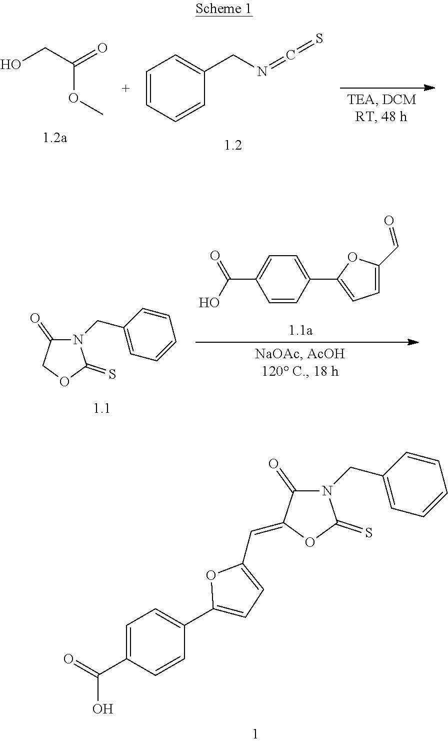

oxo-2-thioxooxazolidin-5-ylidene) methyl) furan-2-yl)benzoic acid (1) as yellow solid. Yield: 0.060 g, 12.2%. MS (ES″) m/z=404.2 [M−1], ¹H NMR (400 MHz, d6-DMSO) δ 13.10 (bs, 1H), 8.01 (dd, 4H, J=37.4, 8.3 Hz), 7.46 (m, 1H), 7.34 (m, 6H), 7.06 (s, 1H), 5.00 (s, 2H)

### Example 2. Preparation of (Z)-4-(5-((1-Benzyl-3-methyl-2,5-dioxoimidazolidin-4-ylidene)methyl)furan-2-yl)benzoic acid (2)

The title compound was prepared according to Scheme 2.

Synthesis of 3-benzylimidazolidine-2,4-dione (2.2). To a solution of imidazolidine-2,4-dione (2.3, 5.00 g, 50 mmol) in N,N-dimethylformamide (100 mL) was added K₂CO₃ (20.7 g, 150 mmol) and the reaction mixture was cooled to 0° C. Bromomethylbenzene (2.3a, 6.5 mL, 55 mmol) was added slowly over 30 min at 0° C. and the reaction mixture was warmed and stirred at room temperature for 18 h. The reaction mixture was quenched with water (200 mL) and the mixture was extracted with ethyl acetate (500 mL). The organic layer was separated, washed with brine, dried over anhydrous sodium sulfate and concentrated under reduced pressure to get the crude product. The crude product was purified by silica gel (230-400 mesh) column chromatography eluting with 40% ethyl acetate in hexanes to afford 3-benzylimidazolidine-2,4-dione (2.2) as an off-white solid. Yield: 3.00 g, 31.0%.

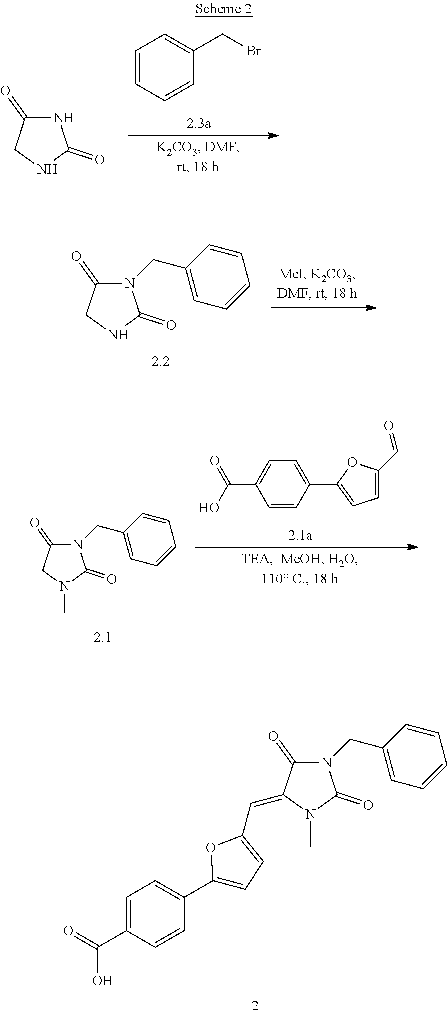 [SMILES: O=C1CNC(=O)N1 ; BrCc1ccccc1 ; O=C1CNC(=O)N1Cc1ccccc1 ; O=Cc1ccc(-c2ccc(C(=O)O)cc2)o1 ; CN1CC(=O)N(Cc2ccccc2)C1=O ; CN1C(=O)N(Cc2ccccc2)C(=O)/C1=C/c1ccc(-c2ccc(C(=O)O)cc2)o1]

Synthesis of 3-benzyl-1-methylimidazolidine-2,4-dione (2.1). To a solution of 3-benzylimidazolidine-2,4-dione (2.2, 1.00 g, 5.26 mmol) in N,N-dimethylformamide (10 mL) was added K₂CO₃ (1.45 g, 10.5 mmol) followed by addition of iodomethane (0.4 mL, 6.31 mmol) at room temperature. The reaction mixture was stirred at room temperature for 18 h. After completion, water (50 mL) was added and the product was extracted with ethyl acetate (100 mL). The organic layer was separated, washed with saturated solution of sodium chloride, dried over anhydrous sodium sulfate and concentrated under reduced pressure. The crude product was purified by silica gel (230-400 mesh) column chromatography eluting with 12% ethyl acetate in hexanes to afford 3-benzyl-1-methylimidazolidine-2,4-dione (2.1) as a yellow oil. Yield: 0.77 g, 72.0%.

Synthesis of (Z)-4-(5-((1-benzyl-3-methyl-2,5-dioxoimidazolidin-4-ylidene)methylguran-2-yl)benzoic acid (2). To a solution of 3-benzyl-1-methylimidazolidine-2, 4-dione (2.1, 0.77 g, 3.77 mmol) in MeOH: H₂O (2:1, 15 mL) was added 4-(5-formylfuran-2-yl) benzoic acid (2.1a, 0.733 g, 3.39 mmol) and triethylamine (1.6 mL, 11.3 mmol) at room temperature. The reaction mixture was heated at 100° C. for 18 h. The reaction was cooled to room temperature, the solvents were removed under reduced pressure and water (20 mL) was added. The pH of reaction mixture was adjusted to ˜6 by addition of citric acid solution and a precipitate formed which was collected by filtration. The product was purified by washing with diethyl ether and methanol to afford (Z)-4-(5-((1-benzyl-3-methyl-2, 5-dioxoimidazolidin-4-ylidene) methyl)furan-2-yl)benzoic acid (2) as a yellow solid.Yield: 0.35 g, 23.2%. MS (ES⁺) m/z=403.2 [M+1], ¹H NMR (400 MHz, d6-DMSO) δ 13.03 (bs, 1H), 7.95 (dd, 4H, J=42.8, 6.6 Hz), 7.75 (bs, 1H), 7.32 (m, 6H), 6.58 (bs, 1H), 4.71 (s, 2H), 3.19 (s, 3H)

### Example 3. Preparation of (Z)-4-(5-((1-Benzyl-2,5-dioxoimidazolidin-4-ylidene)methyl)furan-2-yl)benzoic acid (3)

The title compound was prepared according to Scheme 3.

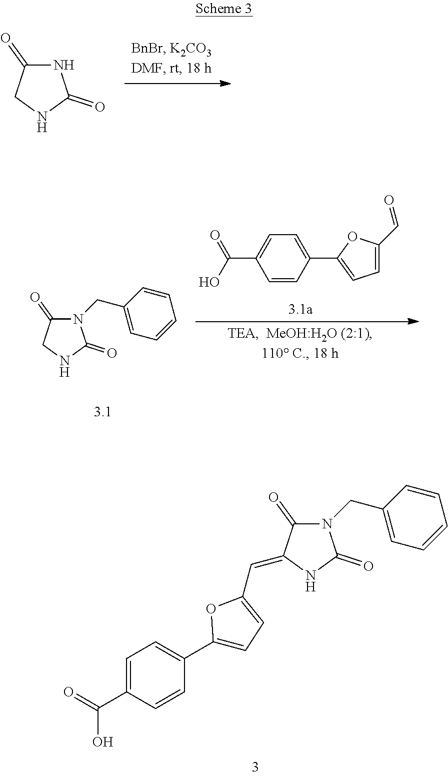 [SMILES: O=C1CNC(=O)N1 ; O=Cc1ccc(-c2ccc(C(=O)O)cc2)o1 ; O=C1CNC(=O)N1Cc1ccccc1 ; O=C(O)c1ccc(-c2ccc(/C=C3\NC(=O)N(Cc4ccccc4)C3=O)o2)cc1]

Synthesis of 3-benzylimidazolidine-2,4-dione (3.1). To a solution of imidazolidine-2,4-dione (5.0 g, 50 mmol) in N,N-dimethylformamide (100 mL) was added K₂CO₃ (20.7 g, 150 mmol) and benzyl bromide (6.5 mL, 55 mmol) at 0° C. The reaction mixture was stirred between 0° C. to room temperature for 18 h. The reaction mixture was quenched with water (200 mL) and product was extracted with ethyl acetate (200 mL). The organic layer was separated, washed twice with a saturated solution of sodium chloride, dried over anhydrous sodium sulfate and concentrated under reduced pressure. The crude product was purified by silica gel (230-400 mesh) column chromatography eluting with 35% ethyl acetate in hexanes and further by washing with diethyl ether and pentane to afford 3-benzylimidazolidine-2,4-dione (3.1) as an off-white solid. Yield: 3.00 g, 31%.

Synthesis of (Z)-4-(5-((1-benzyl-2,5-dioxoimidazolidin-4-ylidene)methylguran-2-yl)benzoic acid (3). To a solution of 3-benzylimidazolidine-2,4-dione (3.1, 0.20 g, 1.05 mmol) in MeOH:H₂O (2:1, 12 ml) was added 4-(5-formylfuran-2-yl)benzoic acid (3.1a, 0.113 g, 0.526 mmol) and triethylamine (0.22 mL, 1.57 mmol) at room temperature. The reaction mixture was heated at 100° C. for 18 h. The reaction mixture was cooled to room temperature and the solvents were removed under reduced pressure. Water (20 mL) was added and the pH of reaction mixture was adjusted to 6 by using a citric acid solution. A precipitate formed and was collected by filtration. The product was purified by washing with diethyl ether and methanol to afford (Z)-4-(5-((1-benzyl-2,5-dioxoimidazolidin-4-ylidene)methyl)furan-2-yl)benzoic acid (3) as a yellow solid. Yield: 0.15 g, 36.7%. MS (ES⁻) m/z=387.1 [M−1], (ES⁺) m/z=389.2 [M+1], ¹H NMR (400 MHz, d6-DMSO) δ 13.13 (bs, 1H), 10.77 (bs, 1H), 8.00 (dd, 4H, J=20.0, 11.7 Hz), 7.37-7.25 (m, 6H), 7.17 (d, 1H, J=3.6), 6.54 (s, 1H), 4.69 (s, 2H)

### Example 4. Preparation of (Z)-4-(5-((1-Benzyl-5-oxo-2-thioxoimidazolidin-4-ylidene)methyl)furan-2-yl)benzoic acid (4)

The title compound was prepared according to Scheme 4.

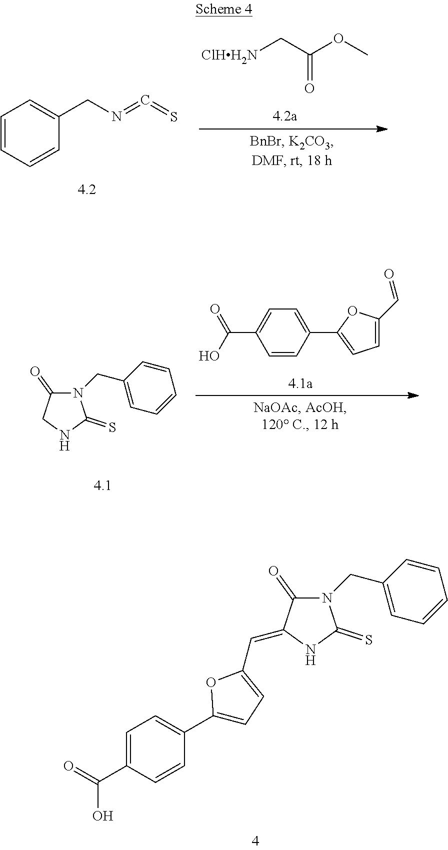

Synthesis of 3-benzyl-2-thioxoimidazolidin-4-one (4.1). To a solution of isothiocyanatomethyl benzene (4.2, 1.0 g, 6.70 mmol) in dichloromethane (15 mL) was added methyl glycinate hydrochloride (0.83 g, 6.70 mmol) and triethylamine (2.4 mL, 16.7 mmol) at 0° C. The reaction mixture was stirred between 0° C. to room temperature for 2 h. The reaction mixture was quenched with water (20 mL) and mixture was extracted with dichloromethane (50 mL). The organic layer was separated, washed with a saturated solution of sodium chloride (2×25 mL), dried over anhydrous sodium sulfate and concentrated under reduced pressure to get the crude product. The crude product was purified by silica gel (230-400 mesh) column chromatography eluting with 20% ethyl acetate in hexanes to afford 3-benzyl-2-thioxoimidazolidin-4-one (4.1) as brown solid. Yield: 0.90 g, 65.2%.

Synthesis of (Z)-4-(5-((1-benzyl-5-oxo-2-thioxoimidazolidin-4-ylidene)methylguran-2-yl)benzoic acid (4). To a solution of methyl 3-benzyl-2-thioxoimidazolidin-4-one (4.1, 0.25 g, 1.21 mmol) in acetic acid (10 mL) was added sodium acetate (0.149 g, 1.82 mmol) and 4-(5-formylfuran-2-yl)benzoic acid (4.1a, 0.262 g, 1.21 mmol) at room temperature. The reaction mixture was heated to 120° C. for 12 h. After completion, water (20 mL) was added and a precipitate formed which was collected by filtration. The product was purified by washing with water, diethyl ether and methanol to afford (Z)-4-(5-((1-benzyl-5-oxo-2-thioxoimidazolidin-4-ylidene)methyl)furan-2-yl)benzoic acid (4) as a yellow solid. Yield: 0.170 g, 34.5%. MS (ES⁻) m/z=403.1 [M−1], (ES⁺) m/z=405.2 [M+1], ¹H NMR (400 MHz, d6-DMSO) δ 12.91 (bs, 1H), 12.41 (bs, 1H), 8.04 (dd, 4H, J=24.0, 8.4 Hz), 8.00-7.87 (m, 1H), 7.41 (dd, 2H, J=14.8, 3.6), 7.31 (m, 5H), 6.62 (s, 1H), 5.03 (s, 2H)

### Example 5. Preparation of (Z)-4-(5-((1-Benzyl-3-methyl-5-oxo-2-thioxoimidazolidin-4-ylidene)methyl)furan-2-yl)benzoic acid (5)

The title compound was prepared according to Scheme 5.

Synthesis of 3-benzyl-1-methyl-2-thioxoimidazolidin-4-one (5.1). To a solution of methyl methylglycinate hydrochloride (5.2a, 0.930 g, 6.69 mmol) in dichloromethane (15 mL), (isothiocyanatomethyl)benzene (5.2, 1.0 g, 6.7 mmol) and triethylamine (1.69 g, 16.7 mmol) were added at 0° C. The mixture was stirred at room temperature for 5 h. After completion, water (30 mL) was added and product was extracted in dichloromethane. The organic layer was dried over anhydrous sodium sulfate and concentrated under reduced pressure. The crude product was purified by washings with diethyl ether and pentane to

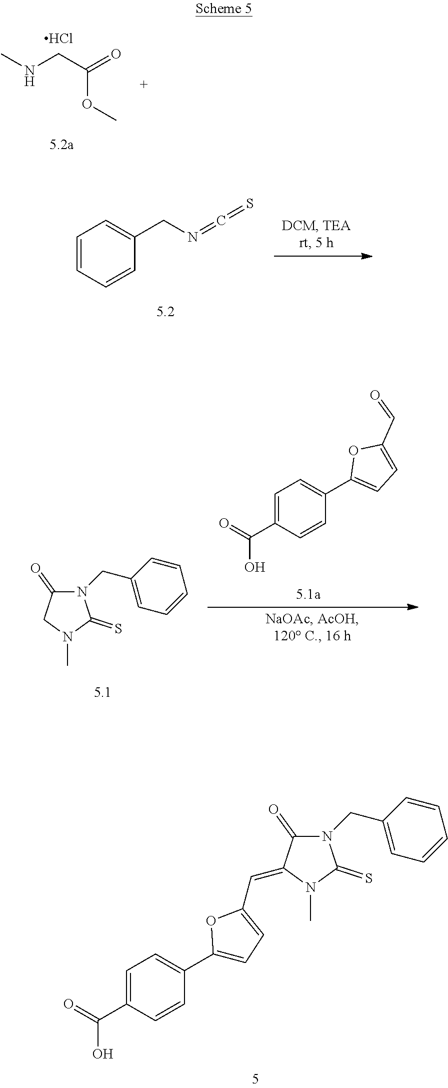

afford 3-benzyl-1-methyl-2-thioxoimidazolidin-4-one (5.1) as brown oil. Yield: 0.60 g, 40.8%.

Synthesis of (Z)-4-(5-((1-benzyl-3-methyl-5-oxo-2-thioxoimidazolidin-4-ylidene)methyl)furan-2-yl)benzoic acid (5). To a solution of 3-benzyl-1-methyl-2-thioxoimidazolidin-4-one (5.1, 0.250 g, 1.13 mmol) in acetic acid (10 mL), sodium acetate (0.139 g, 1.70 mmol) and 4-(5-formylfuran-2-yl)benzoic acid (5.1a, 0.246 g, 1.13 mmol) were added at room temperature. The reaction mixture was heated at 120° C. for 16 h. After completion, reaction mixture was cooled, water (20 mL) was added and the precipitate formed was collected by filtration. The product was purified by washings with water, diethyl ether and methanol to afford (Z)-4-(5-((1-benzyl-3-methyl-5-oxo-2-thioxoimidazolidin-4-ylidene)methyl)furan-2-yl)benzoic acid (5) as yellow solid. Yield: 0.090 g, 18.9%. MS (ES⁻) m/z=417.1 [M−1], ¹H NMR (400 MHz, d6-DMSO) δ 13.05 (bs, 1H), 7.99 (m, 5H), 7.34 (m, 6H), 6.97 (s, 1H), 5.07 (s, 2H), 3.61 (s, 3H).

### Example 6. Preparation of Ethyl 4-(5-((3-benzyl-2,4-dioxoimidazolidin-1-yl)methyl)furan-2-yl)benzoate (6)

The title compound was prepared according to Scheme 6.

Synthesis of ethyl 4-(5-(((2-methoxy-2-oxoethyl)amino)methyl)furan-2-yl)benzoate (6.1). A solution of ethyl 4-(5-formylfuran-2-yl) benzoate (6.2, 1.0 g, 4.3 mmol)

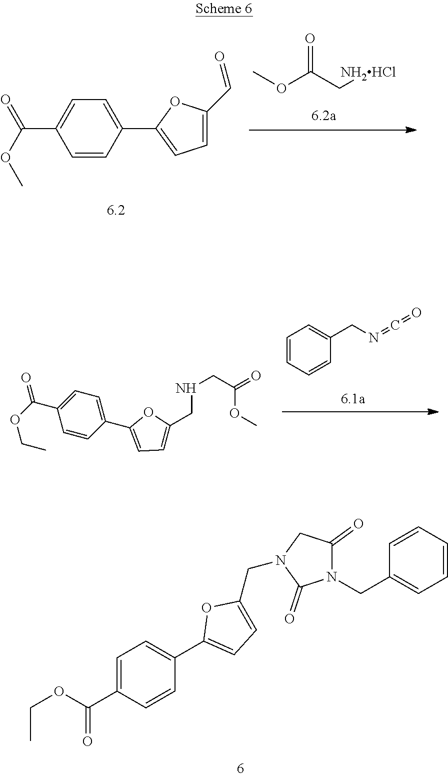

in methanol (100 mL) and methyl glycinate hydrochloride (6.2a, 0.543 g, 4.3 mmol) under nitrogen was treated with triethylamine (0.439 g, 4.3 mmol) at 0 to 5° C. The suspension was stirred at same temperature for 2 h and NaBH₄ (0.164 g, 4.3 mmol) was added in portions. After completion, solvent was removed under reduced pressure to obtain a crude product. The crude product was purified by silica gel (100-200 mesh) column chromatography eluting with 30% ethyl acetate in hexanes to afford ethyl 4-(5-(((2-methoxy-2-oxoethyl)amino)methyl)furan-2-yl)benzoate (6.1). Yield: 0.700 g, 51.09%.

Synthesis of ethyl 4-(5-((3-benzyl-2,4-dioxoimidazolidin-1-yl)methyl)furan-2-yl)benzoate (6). A solution of ethyl 4-(5-(((2-methoxy-2-oxoethyl) amino) methyl) furan-2-yl) benzoate (6.1, 0.70 g, 2.2 mmol) and (isocyanatomethyl) benzene (6.1a, 0.289 g, 2.2 mmol) in dichloromethane (90 mL) was treated with triethylamine (0.44 g, 4.4 mmol) at 0° C. The suspension was stirred for 54 h. After completion, solvent was removed under reduced pressure to get crude. The crude was purified by silica gel (100-200 mesh) column chromatography eluting with 15% ethyl acetate in hexanes to afford ethyl 4-(5-((3-benzyl-2,4-dioxoimidazolidin-1-yl)methyl)furan-2-yl)benzoate (6) Yield: 0.180 g, 19.50%. MS (ES⁺) m/z=419.1 [M+1], ¹H NMR (400 MHz, d6-DMSO) δ 7.88 (dd, 4H, J=74.2, 8.2 Hz) 7.31 (m, 5H), 7.11 (d, 1H, J=3.1 Hz), 6.60 (d, 1H, J=3.1 Hz), 4.61 (s, 2H), 4.58 (s, 2H), 4.32 (q, 2H, J=7.0 Hz), 4.10 (s, 2H), 1.33 (t, 3H, J=7.0 Hz)

### Example 7. Preparation of Ethyl 4-(5-((3-benzyl-2-oxo-4-thioxoimidazolidin-1-yl)methyl)furan-2-yl)benzoate (7)

The title compound was prepared according to Scheme 7.

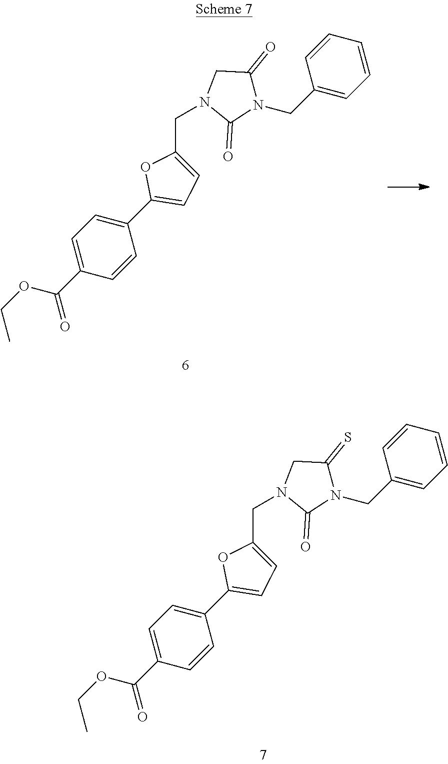 [SMILES: CCOC(=O)c1ccc(-c2ccc(CN3CC(=O)N(Cc4ccccc4)C3=O)o2)cc1 ; CCOC(=O)c1ccc(-c2ccc(CN3CC(=S)N(Cc4ccccc4)C3=O)o2)cc1]

Synthesis of ethyl 4-(5-((3-benzyl-2-oxo-4-thioxoimidazolidin-1-yl)methyl)furan-2-yl)benzoate (7). A solution of ethyl 4-(5-((3-benzyl-2,4-dioxoimidazolidin-1-yl)methyl)furan-2-yl)benzoate (6, 0.10 g, 0.23 mmol) in dioxane (3 mL), Lawesson's reagent (0.096 g, 0.23 mmol) were added. This reaction mixture was heated with stirring at 110° C. for 8 h. Solvents were removed under reduced pressure and the mixture was diluted with water (10 mL) and extracted with dichloromethane (100 mL). The organic layer was dried over anhydrous sodium sulfate and concentrated under reduced pressure to get crude. The crude was purified by silica gel (100-200 mesh) column chromatography eluting with 25% ethyl acetate in hexanes to afford ethyl 4-(5-((3-benzyl-2-oxo-4-thioxoimidazolidin-1-yl)methyl)furan-2-yl)benzoate (7). Yield: 0.020 g, 19.41%. MS (ES⁺) m/z=435.5 [M+1], ¹H NMR (400 MHz, d6-DMSO) δ 7.88 (dd, 4H, J=74.3, 8.3 Hz) 7.30 (m, 5H), 7.12 (d, 1H, J=3.2 Hz), 6.63 (d, 1H, J=3.2 Hz), 4.99 (s, 2H), 4.65 (s, 2H), 4.50 (s, 2H), 4.32 (q, 2H, J=7.0 Hz), 1.33 (t, 3H, J=7.0 Hz)

### Example 8. Preparation of (Z)-4-(5-((3-(4-Fluorobenzyl)-4-oxo-2-thioxooxazolidin-5-ylidene)methyl)furan-2-yl)benzoic acid (8)

The title compound was prepared according to Scheme 8.

Synthesis of 1-fluoro-4-(isothiocyanatomethyl)benzene (8.2). To a solution of (4-fluorophenyl)methanamine (8.3, 5.0 g, 39.96 mmol) in tetrahydrofuran (40 mL) was added carbon disulfide (3.65 g, 47.96 mmol) and triethylamine (4.85 g, 47.96 mmol) at 0° C. The reaction mixture was stirred at 0° C. for 30 minutes (white precipitate formation was observed) and a 1.0 M solution of p-toluene sulfonyl chloride in tetrahydrofuran (40 mL) was

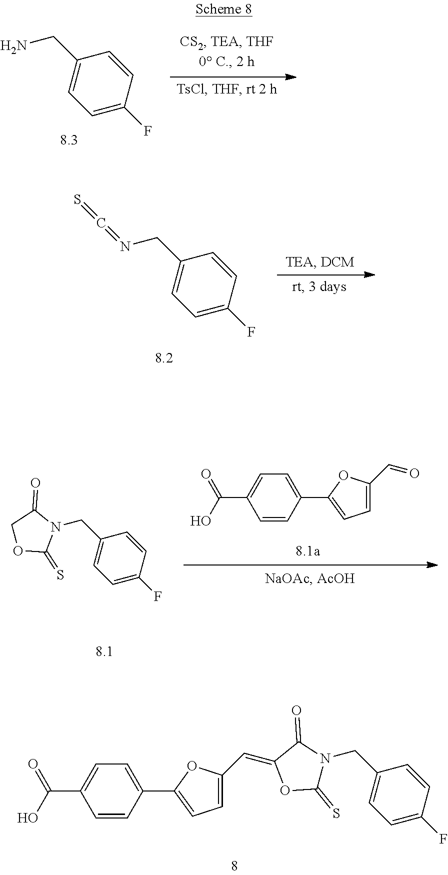 [SMILES: NCc1ccc(F)cc1 ; Fc1ccc(CN=C=S)cc1 ; O=C1COC(=S)N1Cc1ccc(F)cc1 ; O=Cc1ccc(-c2ccc(C(=O)O)cc2)o1 ; O=C(O)c1ccc(-c2ccc(/C=C3\OC(=S)N(Cc4ccc(F)cc4)C3=O)o2)cc1]

added. The reaction mixture was stirred at room temperature for 1 h and 50% HCl in water (100 mL) was added. The mixture was extracted with diethyl ether (250 mL) and the layers were separated. The organic layer was dried over anhydrous sodium sulfate and concentrated under reduced pressure to afford the crude product. The crude product was purified by silica gel (100-200 mesh) column chromatography eluting with hexanes to afford 1-fluoro-4-(isothiocyanatomethyl) benzene (8.2). Yield; 5.12 g, 76.2%.

Synthesis of 3-(4-fluorobenzyl)-2-thioxooxazolidin-4-one (8.1). To a solution of 1-fluoro-4-isothiocyanatomethyl benzene (8.2, 1.2 g, 7.18 mmol) in acetonitrile (30 mL) was added potassium carbonate (2.47 g, 17.90 mmol) in one portion followed by addition of methyl glycolate (0.646 g, 7.18 mmol) at 0° C. The reaction mixture was stirred at room temperature for 16 h. After completion, water (50 mL) was added to the reaction and the mixture was extracted with diethyl ether (200 mL). The organic layer was dried over anhydrous sodium sulfate and concentrated under reduced pressure to get the crude product. The crude product was purified by silica gel (100-200 mesh) column chromatography eluting with 5-10% ethyl acetate in hexanes to afford 3-(4-fluorobenzyl)-2-thioxooxazolidin-4-one (8.1). Yield; 0.160 g, 10%.

Synthesis of (Z)-4-(5-((3-(4-fluorobenzyl)-4-oxo-2-thioxooxazolidin-5-ylidene)methylguran-2-yl)benzoic acid (8). To a solution of 3-(4-fluorobenzyl)-2-thioxooxazolidin-4-one (8.1, 0.180 g, 0.8 mmol) in acetic acid (5 mL), sodium acetate (0.078 g, 0.96 mmol) and 4-(5-formylfuran-2-yl)benzoic acid (8.1a, 0.288 g, 1.29 mmol) were added at room temperature. The reaction mixture was heated at 120° C. for 16 h. After completion, water (15 mL) was added and the precipitate formed was collected by filtration. The product was purified by washings with water, diethyl ether and n-pentane to afford (Z)-4-(5((3-(4-fluorobenzyl)-4-oxo-2-thioxooxazolidin-5-ylidene)methyl)furan-2-yl)benzoic acid (8). Yield: 0.090 g, 26%. MS (ES⁻) m/z=422.0 [M−1], ¹H NMR (400 MHz, d6-DMSO) δ 13.08 (bs, 1H), 7.99 (dd, 4H, J=38.3, 8.2 Hz), 7.45 (m, 3H), 7.30 (d, 1H, J=3.8 Hz), 7.18 (t, 2H, J=9.0 Hz)), 7.05 (s, 1H), 4.99 (s, 2H)

### Example 9. Preparation of (Z)-3′-((3-(4-Fluorobenzyl)-4-oxo-2-thioxooxazolidin-5-ylidene)methyl)-[1,1′-biphenyl]-4-carboxylic acid (9)

The title compound was prepared according to Scheme 9.

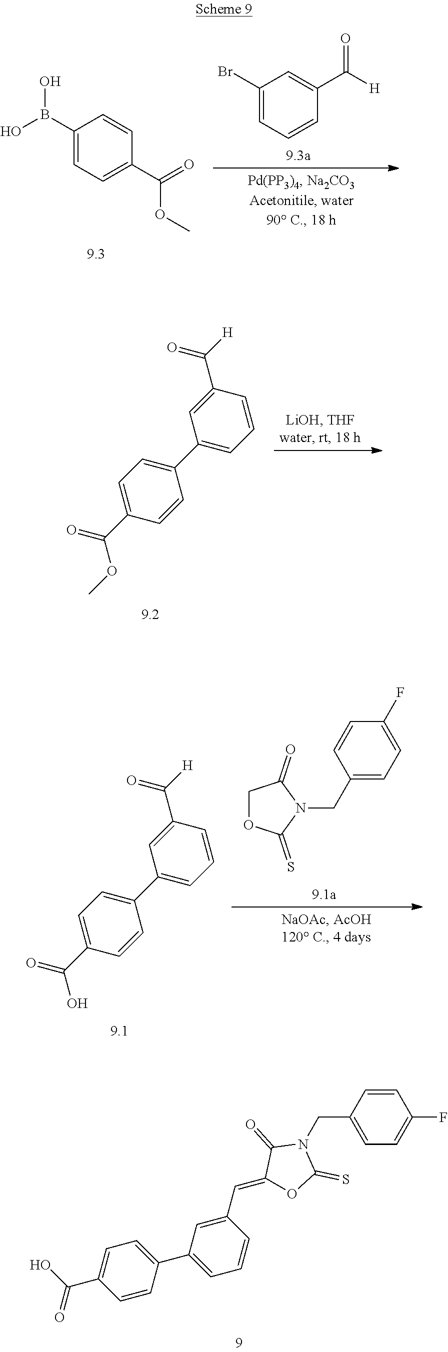

Synthesis of methyl 3′-formyl-[1,1′-biphenyl]-4-carboxylate (9.2). To a solution of (4-(methoxycarbonyl)phenyl)boronic acid (9.3, 3.0 g, 16.66 mmol), Na₂CO₃ (4.4 g, 41.66 mmol) and 3-bromobenzaldehyde (9.3a, 3.08 g 16.66 mmol) in acetonitrile (50 mL), water (10 mL) was added at room temperature. The reaction mixture was purged with argon for 15 minutes and Pd(PPh₃)₄ (0.962 g, 0.833 mmol) was added at room temperature. The reaction mixture was again purged with argon for 10 minutes and heated at 90° C. for 18 h. After completion, reaction mass was cooled to room temperature and solvents was removed under reduced pressure. The reaction mixture was diluted with ice-cold water (100 mL) and extracted with dichloromethane (2×100 mL). The organic layer was separated, dried over anhydrous sodium sulfate and concentrated under reduced pressure. The crude product was purified by silica gel (100-200 mesh) column chromatography eluting with 20% ethyl acetate in hexanes to afford methyl 3′-formyl-[1,1′-biphenyl]-4-carboxylate (9.2) as off-white solid. Yield: 2.0 g, 52%.

Synthesis of 3′-formyl-[1,1′-biphenyl]-4-carboxylic acid (9.1). To a solution of methyl 3′-formyl-[1,1′-biphenyl]-4-carboxylate (9.2, 1.0 g, 4.16 mmol) in tetrahydrofuran (15 mL), LiOH (0.33 g, 8.33 mmol) dissolved in water (5.0 mL) was added at room temperature. The reaction mixture was stirred at room temperature for 18 h. After completion, tetrahydrofuran was removed under reduced pressure, water (30 mL) was added and pH was adjusted to ˜7 by addition of citric acid solution. The product was extracted with dichloromethane (2×50 mL) and the organic layer was separated, dried over anhydrous sodium sulfate and concentrated under reduced pressure to get crude. The crude was purified by silica gel (100-200 mesh) column chromatography eluting with 10-12% MeOH in dichloromethane to afford 3′-formyl-[1,1′-biphenyl]-4-carboxylic acid (9.1) as off white solid. Yield: 0.210 g, 22.3%.

Synthesis of (Z)-3′-((3-(4-fluorobenzyl)-4-oxo-2-thioxooxazolidin-5-ylidene)methyl)-[1,1′-biphenyl]-4-carboxylic acid (9). To a solution of 3′-formyl-[1,1′-biphenyl]-4-carboxylic acid (9.1, 0.200 g, 0.884 mmol) in acetic acid (10 mL), sodium acetate (0.145 g, 1.76 mmol) and 3-(4-fluorobenzyl)-2-thioxooxazolidin-4-one (9.1a, 0.219 g, 0.973 mmol) were added at room temperature. The reaction mixture was heated at 120° C. for 4 days. Cooled the reaction to room temperature, water (20 mL) was added and the precipitate formed was collected by filtration. The product was purified by washings and recrystallization with water, diethyl ether and MeOH to afford (Z)-3′-((3-(4-fluorobenzyl)-4-oxo-2-thioxothiazolidin-5-ylidene)methyl)-[1,1′-biphenyl]-4-carboxylic acid (9) as off white solid. Yield: 0.030 g, 7.8%. MS (ES⁻) m/z=432.0 [M−1], ¹H NMR (400 MHz, d6-DMSO) δ 8.21 (bs, 1H), 7.99 (m, 3H), 7.84 (bs, 1H), 7.75 (bs, 2H), 7.67 (bs, 1H), 7.46 (bs, 2H), 7.19 (bs, 2H), 7.11 (bs, 1H), 5.00 (s, 2H)

### Example 10. Preparation of (Z)-5-(5-((3-(4-Fluorobenzyl)-4-oxo-2-thioxooxazolidin-5-ylidene)methyl)furan-2-yl)picolinate (10)

The title compound was prepared according to Scheme 10.

Synthesis of methyl 5-(5-formylfuran-2-yl)picolinate (10.1). To a solution of methyl 5-bromopicolinate (10.2, 2.5 g, 11.66 mmol) in dioxane (50 mL), (5-formylfuran-2-yl)boronic acid (10.2a, 2.4 g, 17.4 mmol) and Cs₂CO₃ (7.50 g, 23.2 mmol) in water (5.0 mL)

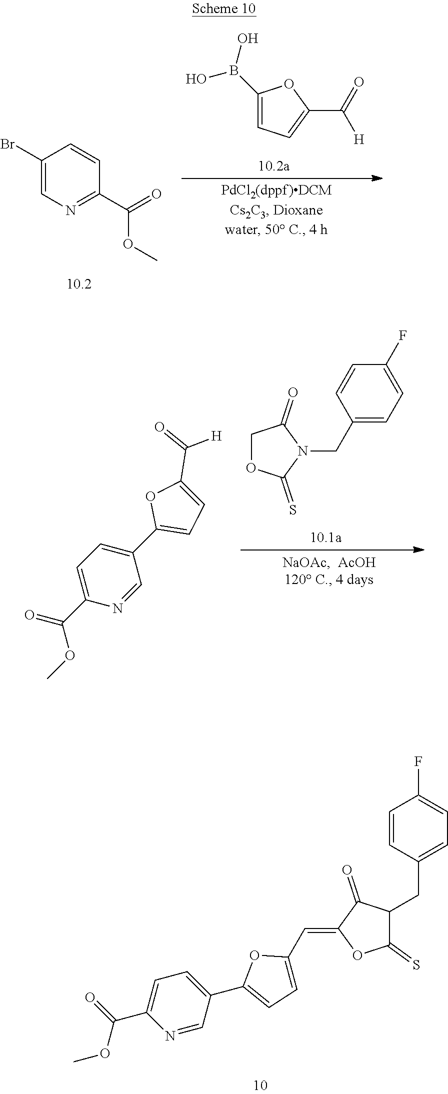

were added at room temperature. The reaction mixture was degassed for 15 min with argon and PdCl₂(dppf)·CH₂Cl₂ catalyst (0.95 g) was added under argon. The reaction mixture was then stirred at 50° C. for 4 h. The reaction mixture was cooled to room temperature, solvent was removed under reduced pressure and water (100 mL) was added. The product was extracted with dichloromethane and organic layer was separated, dried over anhydrous sodium sulfate and concentrated under reduced pressure. The crude product was purified by silica gel (230-400 mesh) column chromatography eluting with 5-10% MeOH in dichloromethane to afford methyl 5-(5-formylfuran-2-yl)picolinate (10.1) as off white solid. Yield: 0.530 g, 20.0%.

Synthesis of methyl (Z)-5-(5-((3-(4-fluorobenzyl)-4-oxo-2-thioxooxazolidin-5-ylidene)methyl)furan-2-yl)picolinate (10). To a solution of methyl 5-(5-formylfuran-2-yl)picolinate (10.1, 0.200 g, 0.865 mmol) in acetic acid (10 mL), sodium acetate (0.106 g, 1.29 mmol) and 3-(4-fluorobenzyl)-2-thioxooxazolidin-4-one (10.1a, 0.214 g, 0.952 mmol) were added at room temperature. The reaction mixture was heated at 120° C. for 4 days. After completion, cooled reaction mixture, water (20 mL) was added and the precipitate formed was collected by filtration. The product was purified by silica gel (100-200 mesh) column chromatography eluting with 20-30% ethyl acetate in hexanes and further washings with diethyl ether to afford methyl (Z)-5-(5-((3-(4-fluorobenzyl)-4-oxo-2-thioxooxazolidin-5-ylidene)methyl)furan-2-yl)picolinate (10) as yellow solid. Yield: 0.090 g, 23.7%. MS (ES⁺) m/z=439.1 [M+1], ¹H NMR (400 MHz, d6-DMSO) δ 9.17 (bs, 1H), 8.33 (bs, 1H), 8.17 (bs, 1H), 7.60 (bs, 1H), 7.45 (bs, 2H), 7.32 (bs, 1H), 7.19 (bs, 2H), 7.07 (bs, 1H), 4.99 (s, 2H), 3.90 (s, 3H).

### Example 11. Preparation of 4-(5-((3-Benzyl-2,4-dioxoimidazolidin-1-yl)methyl)furan-2-yl)benzoic acid (11)

The title compound was prepared according to Scheme 11.

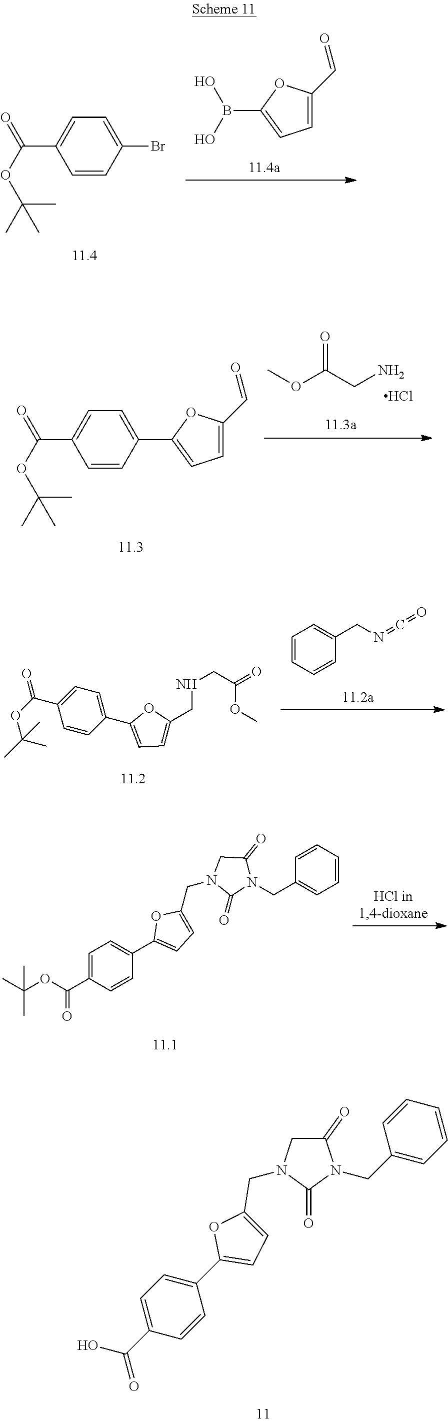

Synthesis of tert-butyl 4-(5-formylfuran-2-yl)benzoate (11.3). To a solution of tert-butyl 4-bromobenzoate (11.4, 5.0 g, 19.4 mmol) and (5-formylfuran-2-yl)boronic acid (11.4a, 2.72 g 19.4 mmol) in toluene (50 mL) and ethanol (50 mL), water (20 mL) was added at room temperature. The reaction mixture was purged with argon for 15 minutes and PdCl₂ (PPh₃)₂ (0.68 g, 0.97 mmol), sodium carbonate (5.12 g, 48.6 mmol) were added. The mixture was again purged with argon for 10 minutes and heated with stirring at 90° C. for 16 h. After completion, solvent was removed under reduced pressure and the mixture was diluted with water (40 mL) and extracted with dichloromethane (500 mL). The organic layer was dried over anhydrous sodium sulfate and concentrated under reduced pressure to get crude. The crude was purified by silica gel (100-200 mesh) column chromatography eluting with 40% ethyl acetate in hexanes to afford tert-butyl 4-(5-formylfuran-2-yl)benzoate (11.3). Yield: 1.9 g, 36.53%.

Synthesis of tert-butyl 4-(5-(((2-methoxy-2-oxoethyl)amino)methyl)furan-2-yl)benzoate (11.2). To a solution of tert-butyl 4-(5-formylfuran-2-yl) benzoate (11.3, 1.250 g, 4.5 mmol) in methanol (75 mL) and methyl glycinate hydrochloride (11.3a, 0.574 g, 4.5 mmol), triethylamine (0.574 g, 4.5 mmol) was added at 0-5° C. The suspension was stirred at 0-5° C. for 2 h and then NaBH₄ (0.17 g, 4.5 mmol) was added in portions and stirring was continued for 2 h. After completion, solvent was removed under reduced pressure to get crude. The crude was purified by silica gel (100-200 mesh) column chromatography eluting with 35% ethyl acetate in hexanes to afford tert-butyl 4-(5-(((2-methoxy-2-oxoethyl)amino)methyl)furan-2-yl)benzoate (11.2) Yield: 0.725 g, 45.88%;

Synthesis of tert-butyl 4-(5-((3-benzyl-2,4-dioxoimidazolidin-1-yl)methylguran-2-yl)benzoate (11.1). To a solution of tert-butyl 4-(5-(((2-methoxy-2-oxoethyl)amino)methyl)furan-2-yl)benzoate (11.2, 0.700 g, 2.0 mmol) in dichloromethane (75 mL) and (isocyanatomethyl)benzene (11.2a, 0.289 g, 2.4 mmol) under nitrogen was treated with triethylamine (0.410 g, 4.0 mmol) at 0° C. to room temperature for 72 h. After completion, solvent was removed under reduced pressure to get crude. The crude was purified by silica gel (100-200 mesh) column chromatography eluting with 20% ethyl acetate in hexanes to afford tert-butyl 4-(5-((3-benzyl-2,4-dioxoimidazolidin-1-yl)methyl)furan-2-yl)benzoate(11.1). Yield: 0.510 g, 56.41%.

Synthesis of 4-(5-((3-benzyl-2,4-dioxoimidazolidin-1-yl)methylguran-2-yl)benzoic acid (11). To a solution of tert-butyl 4-(5-((3-benzyl-2,4-dioxoimidazolidin-1-yl)methyl)furan-2-yl)benzoate (11.1, 0.150 g, 0.34 mmol) in dioxane (3 mL) under nitrogen was treated with 4 M HCl in dioxane (4.0 mole, 10 mL). The suspension was stirred at room temperature for 16 h. After completion, solvent was removed under reduced pressure to get crude. The crude was purified by washing with diethyl ether and n-pentane to afford 4-(5-((3-benzyl-2,4-dioxoimidazolidin-1-yl)methyl)furan-2-yl)benzoic acid (11). Yield: 0.150 g, 87.71%. MS (ES⁻) m/z=389.0 [M−1], ¹H NMR (400 MHz, d6-DMSO) δ 12.95 (bs, 1H), 7.87 (dd, 4H, J=75.3, 7.8 Hz) 7.30 (m, 5H), 7.09 (bs, 1H), 6.58 (bs, 1H), 4.61 (s, 2H), 4.58 (s, 2H), 4.10 (s, 2H)

### Example 12. Preparation of (Z)-5-(5-((3-Benzyl-4-oxo-2-thioxothiazolidin-5-ylidene)methyl) furan-2-yl)picolinic acid (12)

The title compound was prepared according to Scheme 12.

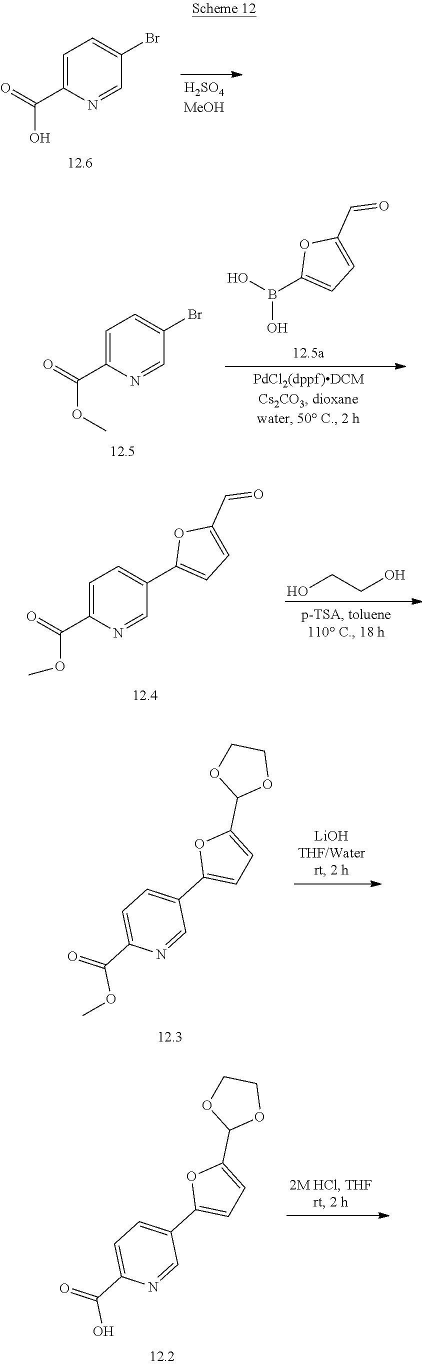

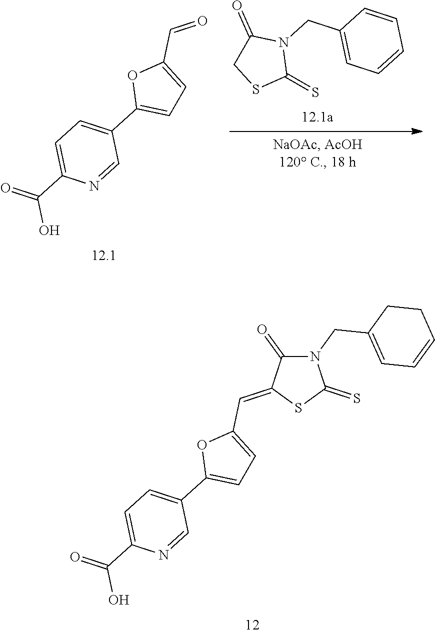

Synthesis of methyl 5-bromopicolinate (12.5). To a solution of 5-bromopicolinic acid (12.6, 6.0 g, 29.9 mmol) in methanol (60 mL), sulfuric acid (3.0 mL) was added at 0° C. and the reaction mixture was heated at 70° C. for 6 h. After completion, cooled the mixture to room temperature and methanol was removed under reduced pressure. To the mixture, water (50 mL) was added and pH was adjusted to ˜6 by a solution of sodium bicarbonate. The product was extracted with ethyl acetate (200 mL), the organic layer was dried over anhydrous sodium sulphate and concentrated under reduced pressure. The crude product was purified by washings with diethyl ether and pentane to afford methyl 5-bromopicolinate (12.5) as white solid. Yield: 6.0 g, 92.0%.

Synthesis of methyl 5-(5-formylfuran-2-yl)picolinate (12.4). To a solution of 5-bromopicolinate (12.5, 2.5 g, 11.66 mmol) in dioxane (50 mL), (5-formylfuran-2-yl)boronic acid (12.5a, 1.03 g, 5.55 mmol) and Cs₂CO₃ (7.50 g, 23.2 mmol) in water (5.0 mL) was added at room temperature. The reaction mixture was degassed for 15 min with argon and PdCl₂(dppf)·CH₂Cl₂ catalyst (0.95 g) was added under argon. The reaction mixture was stirred at 50° C. for 2 h. After completion, reaction mixture was cooled to room temperature, solvents were removed under reduced pressure and water (100 mL) was added. The product was extracted with 10% MeOH in DCM and organic layer was separated, dried over anhydrous sodium sulfate and concentrated under reduced pressure. The crude was purified by silica gel (230-400 mesh) column chromatography eluting with 10% MeOH in DCM to afford methyl 5-(5-formylfuran-2-yl)picolinate(12.4) as off white solid. Yield: 1.20 g, 44.7%.

Synthesis of methyl 5-(5-(1,3-dioxolan-2-yl)furan-2-yl)picolinate (12.3). To a solution of methyl 5-(5-formylfuran-2-yl) picolinate (12.4, 1.0 g, 4.32 mmol) in toluene (20 mL), p-TSA (0.080 g, 0.043 mmol) and ethane-1,2-diol (0.36 mL, 6.49 mmol) were added at room temperature. The reaction mixture was heated at 120° C. for 18 h. After completion, toluene was removed under reduced pressure and water (30 mL) was added. The product was extracted with 10% MeOH in DCM and organic layer was separated, dried over anhydrous sodium sulfate and concentrated under reduced pressure. The product was purified by washings with diethyl ether and n-pentane to afford methyl 5-(5-(1,3-dioxolan-2-yl)furan-2-yl)picolinate (12.3). Yield: 1.0 g, 84%.

Synthesis of 5-(5-(1,3-dioxolan-2-yl)furan-2-yl)picolinic acid (12.2). To a solution of methyl 5-(5-(1,3-dioxolan-2-yl)furan-2-yl)picolinate (12.3, 1.0 g, 3.63 mmol) in THF (15 mL), lithium hydroxide (0.763 g, 18.18 mmol) dissolved in water (2.0 mL) was added at room temperature and stirred for 2 h. After completion, solvent was removed under reduced pressure, water (30 mL) was added and pH was adjusted to ˜7 by citric acid. The precipitated solid was collected by filtration and the product was purified by washings with diethyl ether and MeOH to afford 5-(5-(1,3-dioxolan-2-yl)furan-2-yl)picolinic acid (12.2) as brown solid.Yield: 0.650 g, 68.4%.

Synthesis of 5-(5-formylfuran-2-yl)picolinic acid (12.1). To a solution of 5-(5-(1,3-dioxolan-2-yl)furan-2-yl)picolinic acid (12.2, 1.0 g, 3.63 mmol) in tetrahydrofuran (20 mL), 2 M HCl (10 mL) was added at room temperature and the mixture was stirred for 2 h. After completion, solvent was removed under reduced pressure and the product was purified by washings with diethyl ether and methanol to afford 5-(5-formylfuran-2-yl)picolinic acid (12.1) as brown solid. Yield: 0.50 g, 92.0%.

Synthesis of (Z)-5-(5-((3-benzyl-4-oxo-2-thioxothiazolidin-5-ylidene)methyl)furan-2-yl)picolinic acid (12). To a solution of 5-(5-formylfuran-2-yl)picolinic acid (12.1, 0.30 g, 1.38 mmol) in acetic acid (10 mL), sodium acetate (0.170 g, 2.07 mmol) and 3-benzyl-2-thioxothiazolidin-4-one (12.1a, 0.308 g, 1.38 mmol) were added at room temperature. The reaction mixture was heated at 120° C. for 18 h. After completion, water (20 mL) was added and the precipitate formed was collected by filtration. The product was purified by washings with water, diethyl ether and methanol to afford (Z)-5-(5-((3-benzyl-4-oxo-2-thioxothiazolidin-5-ylidene)methyl)furan-2-yl)picolinic acid (12) as dark brown solid. Yield: 0.230 g, 39.45%. MS (ES⁻) m/z=420.9 [M−1], (ES⁺) m/z=423.1 [M+1], ¹H NMR (400 MHz, d6-DMSO) δ 13.33 (bs, 1H), 9.19 (s, 1H), 8.28 (dd, 2H, J=54.6, 6.6 Hz), 7.77 (s, 1H), 7.64 (bs, 1H), 7.45 (bs, 1H), 7.33 (m, 5H), 5.26 (s, 2H).

### Example 13. Preparation of Methyl (Z)-5-(5-((3-benzyl-4-oxo-2-thioxothiazolidin-5-ylidene)methyl)furan-2-yl)picolinate (13)

The title compound was prepared according to Scheme 13.

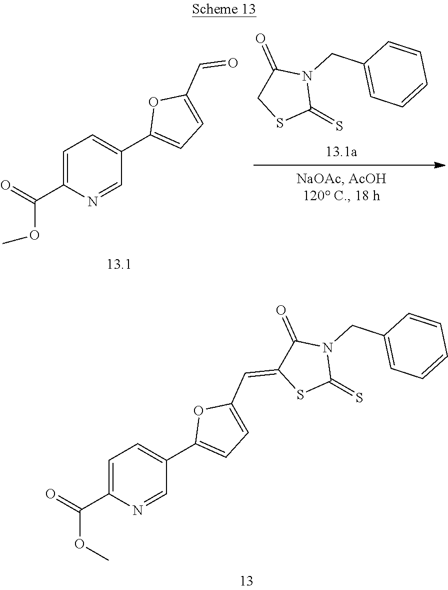

To a solution of methyl 5-(5-formylfuran-2-yl)picolinate (13.1, 0.10 g, 0.448 mmol) in acetic acid (10 mL), sodium acetate (0.055 g, 0.672 mmol) and 3-benzyl-2-thioxothiazolidin-4-one (13.1a, 0.099 g, 0.448 mmol) were added at room temperature. The reaction mixture was heated at 120° C. for 4 h. After completion, water (20 mL) was added and the precipitate formed was collected by filtration. The product was purified by silica gel (230-400 mesh) column chromatography eluting with 5% MeOH in DCM and further washings with diethyl ether and pentane to afford (Z)-5-(5-((3-benzyl-4-oxo-2-thioxothiazolidin-5-ylidene)methyl)furan-2-yl)picolinate (13) as brown solid. Yield: 0.040 g, 21.2%. MS (ES⁺) m/z=437.4 [M+1], ¹H NMR (400 MHz, d6-DMSO) δ 9.20 (s, 1H), 8.30 (dd, 2H, J=49.8, 7.0 Hz), 7.78 (s, 1H), 7.66 (bs, 1H), 7.46 (bs, 1H), 7.33 (m, 5H), 5.26 (s, 2H), 3.91 (s, 3H)

### Example 14. Preparation of (Z)-6-(5-((3-Benzyl-4-oxo-2-thioxothiazolidin-5-ylidene)methyl)furan-2-yl)nicotinic acid (14)

The title compound was prepared according to Scheme 14.

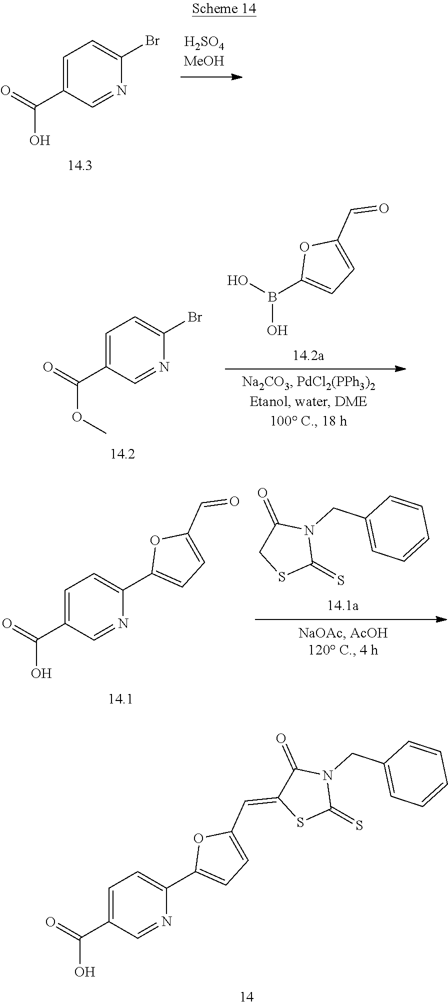

Synthesis of methyl 6-bromonicotinate (14.2). To a solution of 6-bromonicotinic acid (14.3, 2.0 g, 9.90 mmol) in methanol (20 mL), sulfuric acid (2.0 mL) was added at 0° C. and the reaction mixture was heated at 70° C. for 3 h. After completion, the reaction mixture was cooled to room temperature and solvent was removed under reduced pressure. The crude was diluted with water (50 mL) and pH was adjusted to ˜6 by saturated solution of sodium bicarbonate. The reaction mixture was extracted with ethyl acetate (100 mL), organic layer was dried over anhydrous sodium sulphate and concentrated under reduced pressure to get crude. The crude was purified by washings with diethyl ether and pentane to afford methyl 6-bromonicotinate (14.2) as yellow solid. Yield: 1.10 g, 52.0%.

Synthesis of 6-(5-formylfuran-2-yl)nicotinic acid (14.1). To a solution of methyl 6-bromonicotinate (14.2, 1.0 g, 4.62 mmol) in ethanol (10 mL) and dimethoxy ethanol (10 mL), (5-formylfuran-2-yl)boronic acid (14.2a, 0.77 g, 5.5 mmol) and Na₂CO₃ (1.22 g, 11.5 mmol) in water (1.0 mL) was added at room temperature. The reaction mixture was degassed for 15 min with argon and added PdCl₂(PPh₃)₂ (0.16 g, 0.23 mmol) was added under argon atmosphere. The reaction was heated at 100° C. for 18 h. After completion, the reaction was cooled at room temperature, solvent was removed under reduced pressure and water (100 mL) was added. The pH of the reaction mixture was adjusted to ˜6 by saturated solution of citric acid. The product was extracted in 10% MeOH in DCM and organic layer was separated, dried over anhydrous sodium sulfate and concentrated under reduced pressure to get crude. The crude was purified by silica gel (230-400 mesh) column chromatography eluting with 5-10% MeOH in DCM to afford 6-(5-formylfuran-2-yl)nicotinic acid (14.1) as brown color solid. Yield: 0.30 g, 30.0%.

Synthesis of (Z)-6-(5-((3-benzyl-4-oxo-2-thioxothiazolidin-5-ylidene)methyl)furan-2-yl)nicotinic acid (14). To a solution of 6-(5-formylfuran-2-yl)nicotinic acid (14.1, 0.150 g, 0.69 mmol) in acetic acid (10 mL), sodium acetate (0.085 g, 1.03 mmol) and 3-benzyl-2-thioxothiazolidin-4-one (14.1a, 0.154 g, 0.69 mmol) were added at room temperature. The reaction mixture was heated at 120° C. for 4 h. After completion, water (20 mL) was added and the precipitate formed was collected by filtration. The product was purified by washings with water, diethyl ether and methanol to afford (Z)-6-(5-((3-benzyl-4-oxo-2-thioxothiazolidin-5-ylidene)methyl)furan-2-yl)nicotinic acid (14) as brown solid. Yield: 0.095 g, 32.0%. MS (ES⁺) m/z=423.3 [M+1], ¹H NMR (400 MHz, d6-DMSO) δ 13.50 (bs, 1H), 9.13 (s, 1H), 8.49 (d, 1H, J=6.7 Hz), 7.97 (d, 1H, J=8.0 Hz), 7.79 (s, 1H), 7.54 (bs, 1H), 7.46 (bs, 1H), 7.33 (m, 5H), 5.26 (s, 2H)

### Example 15. Preparation of (Z)-5-(5-((3-(4-Fluorobenzyl)-4-oxo-2-thioxothiazolidin-5-ylidene)methyl)furan-2-yl)picolinic acid (15)

The title compound was prepared according to Scheme 15.

Synthesis of (Z)-5-(5-((3-(4-fluorobenzyl)-4-oxo-2-thioxothiazolidin-5-ylidene)methyl)furan-2-yl)picolinic acid (15). To a solution of methyl 5-(5-formylfuran-2-yl)picolinic acid (15.1, 0.150 g, 0.691 mmol) in acetic acid (10 mL), sodium acetate (0.085 g, 1.03 mmol) and 3-(4-fluorobenzyl)-2-thioxothiazolidin-4-one (15.1a, 0.182 g, 0.760 mmol) were added at room temperature. The reaction mixture was heated at 120° C. for 18 h. After completion, water (20 mL) was added and the precipitate formed was collected by filtration. The product was purified by washings with diethyl ether and methanol to afford (Z)-5-(5-((3-

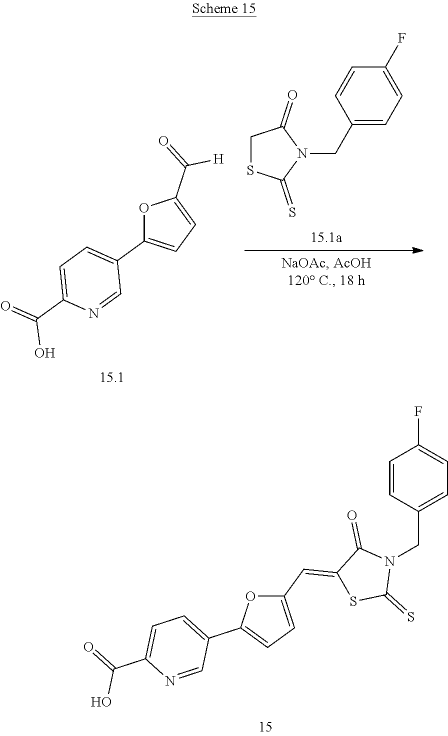

(4-fluorobenzyl)-4-oxo-2-thioxothiazolidin-5-ylidene)methyl)furan-2-yl)picolinic acid (15) as brown solid. Yield: 0.060 g, 20.0%. MS (ES⁺) m/z=438.9 [M+1], ¹H NMR (400 MHz, d6-DMSO) δ 13.34 (bs, 1H), 9.18 (s, 1H), 8.34 (bs, 1H), 8.21 (bs, 1H), 7.77 (s, 1H), 7.64 (bs, 1H), 7.44 (bs, 1H), 7.40 (bs, 2H), 7.18 (bs, 2H), 5.23 (s, 2H)

### Example 16. Preparation of Methyl (Z)-5-(5-((3-(4-fluorobenzyl)-4-oxo-2-thioxothiazolidin-5-ylidene)methyl)furan-2-yl)picolinate (16)

The title compound was prepared according to Scheme 16.

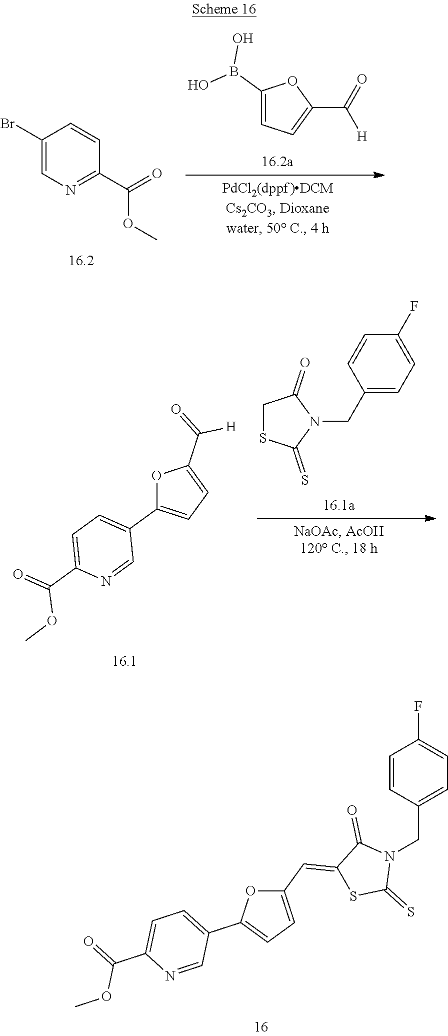

Synthesis of methyl 5-(5-formylfuran-2-yl)picolinate (16.1). To a solution of methyl 5-bromopicolinate (16.2, 2.5 g, 11.66 mmol) in dioxane (50 mL), (5-formylfuran-2-yl)boronic acid (16.2a, 2.4 g, 17.4 mmol) and Cs₂CO₃ (7.50 g, 23.2 mmol) in water (5.0 mL) was added at room temperature. The reaction mixture was degassed with argon for 15 min and PdCl₂(dppf)·CH₂Cl₂ (0.95 g) was added and reaction mixture was heated at 50° C. for 4 h. Cooled the reaction mixture at room temperature, solvent was removed under reduced pressure and water (100 mL) was added. The product was extracted in dichloromethane and organic layer was separated, dried over anhydrous sodium sulfate and concentrated under reduced pressure. The crude was purified by silica gel (230-400 mesh) column chromatography eluting with 5-10% MeOH in dichloromethane to afford methyl 5-(5-formylfuran-2-yl)picolinate (16.1) as off white solid. Yield: 0.530 g, 20.0%.

Synthesis of methyl (Z)-5-(5-((3-(4-fluorobenzyl)-4-oxo-2-thioxothiazolidin-5-ylidene)methyl)furan-2-yl)picolinate (16). To a solution of methyl 5-(5-formylfuran-2-yl)picolinate (16.1, 0.200 g, 0.833 mmol) in acetic acid (10 mL), sodium acetate (0.102 g, 1.24 mmol) and 3-(4-fluorobenzyl)-2-thioxooxazolidin-4-one (16.1a, 0.219 g, 0.973 mmol) were added at room temperature. The reaction mixture was heated at 120° C. for 18 h. After completion, reaction mixture was cooled, water (20 mL) was added and the precipitate formed was collected by filtration. The product was purified by washings with water, diethyl ether and MeOH to afford methyl (Z)-5-(5-((3-(4-fluorobenzyl)-4-oxo-2-thioxothiazolidin-5-ylidene)methyl)furan-2-yl)picolinate (16) as brown solid. Yield: 0.230 g, 58.5%. MS (ES⁺) m/z=455.0 [M+1], ¹H NMR (400 MHz, d6-DMSO) δ 9.19 (s, 1H), 8.29 (dd, 2H, J=48.7, 7.9 Hz), 7.77 (s, 1H), 7.55 (dd, 2H, J=82.4, 3.5 Hz), 7.40 (m, 3H), 7.18 (t, 2H, J=8.7 Hz) 5.23 (s, 2H), 3.91 (s, 3H)

### Example 17. Preparation of (Z)-3′-((3-Benzyl-4-oxo-2-thioxothiazolidin-5-ylidene)methyl)-[1,1′-biphenyl]-4-carboxylic acid (17)

The title compound was prepared according to Scheme 17.

Synthesis of methyl 3′-formyl-[1,1′-biphenyl]-4-carboxylate (17.2b). To a solution of (4-(methoxycarbonyl)phenyl)boronic acid (17.2, 1.0 g, 5.55 mmol) in acetonitrile (10 mL) was added 3-bromobenzaldehyde (17.2a, 1.02 g, 5.55 mmol) and sodium carbonate (1.47 g, 13.8 mmol) at room temperature. The reaction mixture was degassed for 15 min with argon and Pd(PPh₃)₄ (641 mg, 0.555 mmol, 0.1 eq) was added followed by degassing for 5 min. The reaction mixture was then heated at 90° C. for 18 h. After completion, reaction

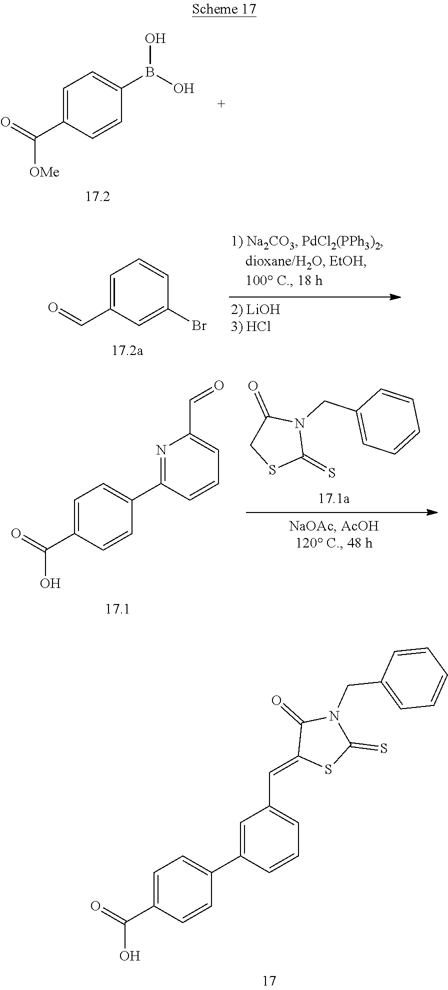

mixture was cooled to room temperature and the acetonitrile was removed under reduced pressure. Water (100 mL) was added to the crude mixture, and the mixture was extracted with ethyl acetate. The organic layer was dried over anhydrous sodium sulfate and concentrated under reduced pressure to get the crude product. The crude product was purified by silica gel (230-400 mesh) column chromatography eluting with 15% ethyl acetate in hexanes to afford methyl 3′-formyl-[1,1′-biphenyl]-4-carboxylate (17.2b) as an off-white color solid. Yield: 0.63 g, 47.3%.

Synthesis of 3′-formyl-[1,1′-biphenyl]-4-carboxylic acid (17.1). To a solution of methyl 3′-formyl-[1,1′-biphenyl]-4-carboxylate (17.2b, 0.30 g, 1.25 mmol) in tetrahydrofuran (10 mL) was added LiOH (0.090 g, 3.75 mmol) dissolved in water (1.0 mL) at room temperature and stirred for 18 h. After completion, water (15 mL) was added and the pH of reaction mixture was adjusted to 6 by addition of 1 N HCl. The mixture was extracted with ethyl acetate and solvent was removed under reduced pressure to get the crude product. The crude product was purified by silica gel (230-400 mesh) column chromatography eluting with 50-80% ethyl acetate in hexanes to afford 3′-formyl-[1,1′-biphenyl]-4-carboxylic acid (17.1) as an off-white solid.Yield: 0.10 g, 35.5%.

Synthesis of (Z)-3′-((3-benzyl-4-oxo-2-thioxothiazolidin-5-ylidene)methyl)-[1,1′-biphenyl]-4-carboxylic acid (17). To a solution of 3′-formyl-[1,1′-biphenyl]-4-carboxylic acid (17.1, 0.10 g, 0.44 mmol) in acetic acid (5 mL), was added sodium acetate (0.108 g, 1.32 mmol) and 3-benzyl-2-thioxothiazolidin-4-one (17.1a, 0.098 g, 0.442 mmol) at room temperature. The reaction mixture was heated at 120° C. for 48 h. After completion, water (20 mL) was added and a precipitate formed which was collected by filtration. The product was purified by washing with water, diethyl ether and methanol to afford (Z)-3′-((3-benzyl-4-oxo-2-thioxothiazolidin-5-ylidene)methyl)-[1,1′-biphenyl]-4-carboxylic acid (17) as yellow solid. Yield: 0.12 g, 63.15%. MS (ES⁻) m/z=430.1 [M−1], ¹H NMR (400 MHz, d6-DMSO) δ 13.09 (bs, 1H), 8.04 (bs, 1H), 7.98 (s, 1H), 7.97 (dd, 4H, J=77.9, 8.2 Hz), 7.89 (m, 1H), 7.69 (m, 2H), 7.32 (m, 5H), 5.27 (s, 2H)

### Example 18. Preparation of (Z)-4-(6-((3-Benzyl-4-oxo-2-thioxothiazolidin-5-ylidene)methyl)pyridin-2-yl)benzoic acid (18)

The title compound was prepared according to Scheme 18.

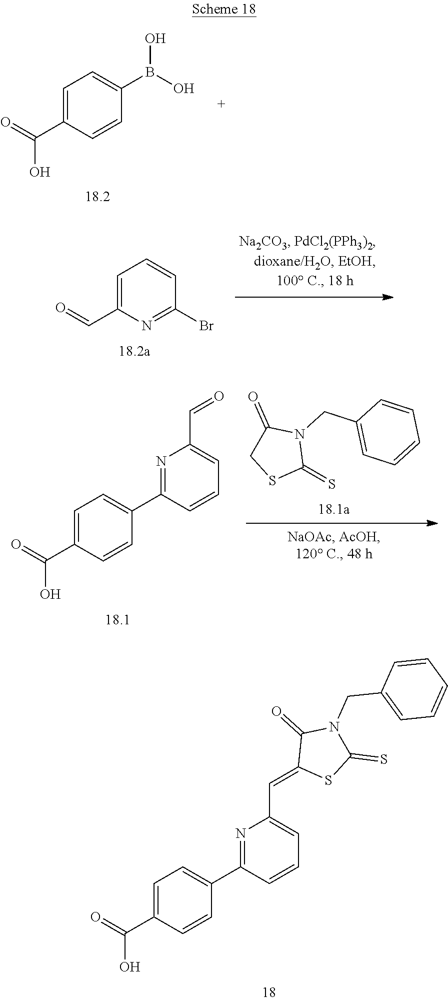

Synthesis of 4-(6-formylpyridin-2-yl)benzoic acid (18.1). To a solution of 4-boronobenzoic acid (18.2, 1.0 g, 6.02 mmol) in ethanol (10 mL) and dioxane (10 mL) was added 6-bromopicolinaldehyde (18.2a, 1.12 g, 6.02 mmol) and a solution of Na₂CO₃ (1.91 g, 18.0 mmol) dissolved in water (5.0 mL) at room temperature. The reaction mixture was degassed with argon for 15 min and PdCl₂(PPh₃)₂ (422 mg, 0.60 mmol, 0.10 eq) was added and followed by degassing for an additional 5 min. The reaction as stirred at 100° C. for 18 h. The reaction mixture was cooled and the solvent was removed under reduced pressure. Water (100 mL) was added and the pH of mixture was adjusted to ˜6 by addition of citric acid solution and the mixture was extracted with ethyl acetate. The organic layer was separated, dried over anhydrous sodium sulfate and concentrated under reduced pressure to get the crude product. The crude product was purified by silica gel (230-400 mesh) column chromatography eluting with 5-10% methanol in dichloromethane to afford 4-(6-formylpyridin-2-yl)benzoic acid (18.1) as an off-white color solid. Yield: 0.110 g, 8.0%.

Synthesis of (Z)-4-(6-((3-benzyl-4-oxo-2-thioxothiazolidin-5-ylidene)methyl)pyridin-2-yl)benzoic acid (18). To a solution of methyl 4-(6-formylpyridin-2-yl)benzoic acid (7.1, 0.10 g, 0.44 mmol) in acetic acid (10 mL) was added sodium acetate (0.054 g, 0.66 mmol) and 3-benzyl-2-thioxothiazolidin-4-one (18.1a, 0.108 g, 0.48 mmol) at room temperature. The reaction mixture was heated at 120° C. for 6 h. After completion, water (20 mL) was added and a precipitate formed which was collected by filtration. The product was purified by washing with diethyl ether and pentane to afford (Z)-4-(6-((3-benzyl-4-oxo-2-thioxothiazolidin-5-ylidene)methyl)pyridin-2-yl)benzoic acid (18) as a yellow solid. Yield: 0.110 g, 57.8%. MS (ES⁻) m/z=433.2 [M−1], ¹H NMR (400 MHz, d6-DMSO) δ 13.09 (bs, 1H), 8.21 (dd, 4H, J=63.0, 8.2 Hz), 8.11 (m, 2H), 7.99 (m, 1H), 7.97 (2, 1H), 7.31 (m, 5H), 5.28 (s, 2H)

### Example 19. Preparation of (Z)-4-(4-((3-Benzyl-4-oxo-2-thioxothiazolidin-5-ylidene)methyl)pyrimidin-2-yl)benzoic acid (19)

The title compound was prepared according to Scheme 19.

Synthesis of 4-(4-methylpyrimidin-2-yl)benzoic acid (19.2). To a solution of 4-boronobenzoic acid (19.3, 3.90 g, 23.43 mmol) in dimethoxy ethanol (50 mL), 2-chloro-4-methylpyrimidine (19.3a, 2.50 g, 19.53 mmol) and Na₂CO₃ (5.10 g, 48.8 mmol) in water (10.0 mL) was added at room temperature. The reaction mixture was degassed for 15 min with argon and Pd(PPh₃)₄ (3.3 g, 2.92 mmol) was added under argon and heated the reaction mixture at 100° C. for 18 h. After completion, cooled the reaction mixture at room temperature, and solvent was removed under reduced pressure. Water (100 mL) was added and the pH was adjusted to ˜6 by citric acid solution. The product was extracted in 10% MeOH in DCM and organic layer was separated, dried over anhydrous sodium sulfate and concentrated under reduced pressure to get crude. The crude was purified by washings with

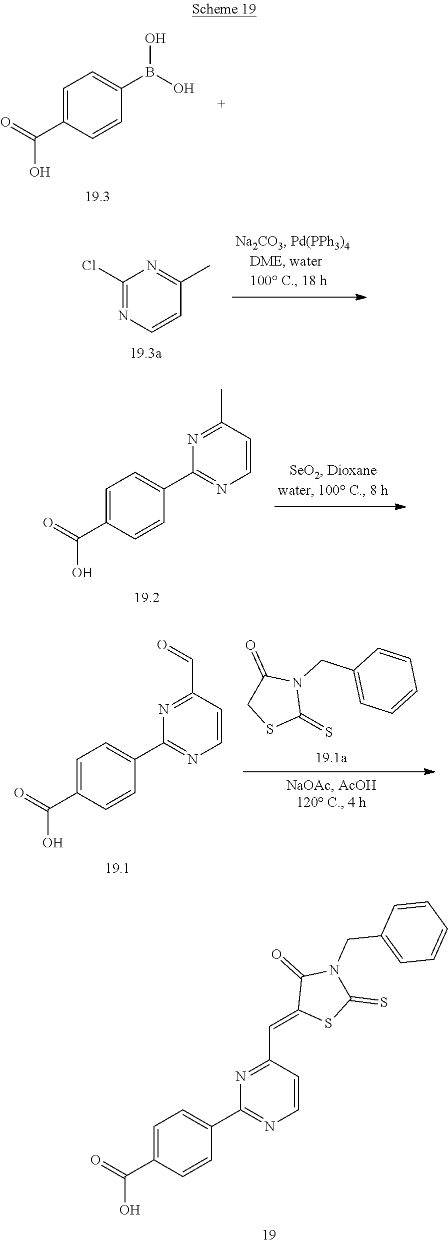

diethyl ether to afford 4-(4-methylpyrimidin-2-yl)benzoic acid (19.2) as off white solid. Yield: 0.810 g (Crude)

Synthesis of 4-(4-formylpyrimidin-2-yl)benzoic acid (19.1). To a solution of 4-(4-methylpyrimidin-2-yl)benzoic acid (19.2, 0.60 g, 2.80 mmol) in dioxane (20 mL), water (0.2 mL) was added at room temperature. The reaction mixture was stirred at 100° C. for 4 h. After completion, cooled the reaction mixture to room temperature and solvent was removed under reduced pressure to get crude. The crude was purified by silica gel (230-400 mesh) column chromatography eluting with 5-10% MeOH in DCM to afford 4-(4-formylpyrimidin-2-yl)benzoic acid(19.1) as off-white solid. Yield: 0.180 g, 28.1%.

Synthesis of (Z)-4-(4-((3-benzyl-4-oxo-2-thioxothiazolidin-5-ylidene)methyl)pyrimidin-2-yl)benzoic acid (19). To a solution of 4-(4-formylpyrimidin-2-yl)benzoic acid (19.1, 0.180 g, 0.789 mmol) in acetic acid (10 mL), sodium acetate (0.097 g, 1.18 mmol) and 3-benzyl-2-thioxothiazolidin-4-one (19.1a, 0.176 g, 0.789 mmol) were added at room temperature. The reaction mixture was heated at 120° C. for 4 h. After completion, water (20 mL) was added and the precipitate formed was collected by filtration. The product was purified by washings with water, diethyl ether and methanol to afford (Z)-4-(4-((3-benzyl-4-oxo-2-thioxothiazolidin-5-ylidene)methyl)pyrimidin-2-yl)benzoic acid (19) as yellow solid. Yield: 0.098 g, 28.7%. MS (ES⁺) m/z=434.4 [M+1], ¹H NMR (400 MHz, d6-DMSO) δ 13.21 (bs, 1H), 9.13 (d, 1H, J=4.5 Hz), 8.37 (dd, 4H, J=157.7, 7.8 Hz), 7.98 (d, 1H, J=4.5 Hz), 7.94 (s, 1H), 7.34 (m, 5H), 5.28 (s, 2H)

### Example 20. Preparation of (Z)-3′-((3-(4-Fluorobenzyl)-4-oxo-2-thioxothiazolidin-5-ylidene)methyl)-[1,1′-biphenyl]-4-carboxylic acid (20)

The title compound was prepared according to Scheme 20.

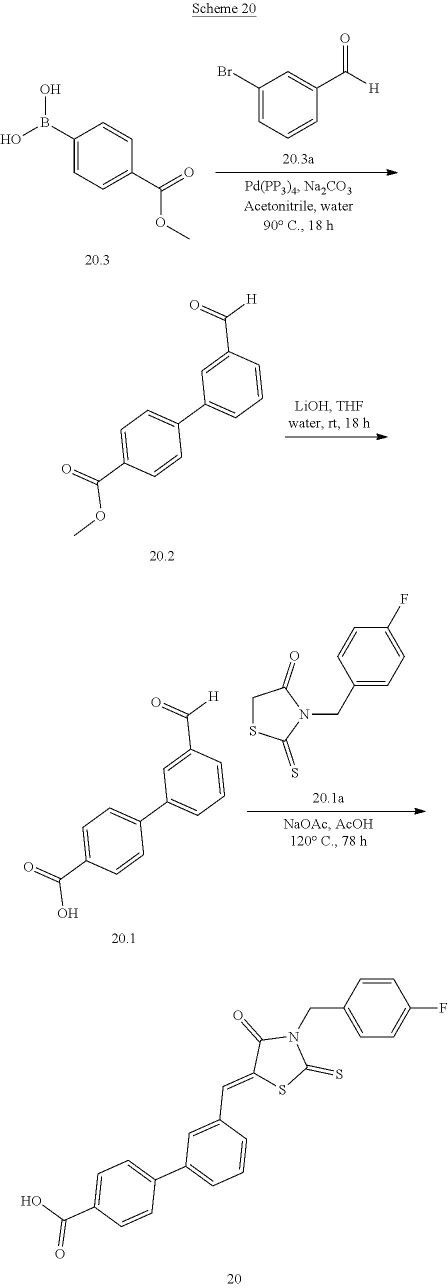

Synthesis of methyl 3′-formyl-[1,1′-biphenyl]-4-carboxylate (20.2). To a solution of (4-(methoxycarbonyl)phenyl)boronic acid (20.3, 3.0 g, 16.66 mmol), Na₂CO₃ (4.4 g, 41.66 mmol) and 3-bromobenzaldehyde (20.3a, 3.08 g 16.66 mmol) in acetonitrile (50 mL), water (10 mL) was added at room temperature. The reaction mixture was purged with argon for 15 minutes and Pd(PPh₃)₄ (0.962 g, 0.833 mmol) was added at room temperature. The reaction mixture was again purged with argon gas for 10 minutes and heated at 90° C. for 18 h. After completion, reaction mass was cooled to room temperature and solvent was removed under reduced pressure. The reaction mixture was diluted with ice-cold water (100 mL) and extracted with dichloromethane (2×100 mL). The organic layer was separated, dried over anhydrous sodium sulfate and concentrated under reduced pressure. The crude was purified by silica gel (100-200 mesh) column chromatography eluting with 20% ethyl acetate in hexanes to afford methyl 3′-formyl-[1,1′-biphenyl]-4-carboxylate (20.2) as off-white solid. Yield: 2.0 g, 52%.

Synthesis of 3′-formyl-[1,1′-biphenyl]-4-carboxylic acid (20.1). To a solution of methyl 3′-formyl-[1,1′-biphenyl]-4-carboxylate (20.2, 1.0 g, 4.16 mmol) in THF (15 mL), LiOH (0.33 g, 8.33 mmol) dissolved in water (5.0 mL) was added at room temperature. The reaction mixture was stirred at room temperature for 18 h. After completion, THF was removed under reduced pressure, water (30 mL) was added and pH was adjusted to ˜7 by addition of citric acid solution. The product was extracted with dichloromethane (100 mL) and the organic layer was separated, dried over anhydrous sodium sulfate and concentrated under reduced pressure. The crude was purified by silica gel (100-200 mesh) column chromatography eluting with 10-12% MeOH in dichloromethane to afford 3′-formyl-[1,1′-biphenyl]-4-carboxylic acid (20.1) as off white solid.Yield: 0.210 g, 22.3%.

Synthesis of (Z)-3′-((3-(4-fluorobenzyl)-4-oxo-2-thioxothiazolidin-5-ylidene)methyl)-[1,1′-biphenyll-4-carboxylic acid (20). To a solution of 3′-formyl-[1,1′-biphenyl]-4-carboxylic acid (20.1, 0.10 g, 0.442 mmol) in acetic acid (10 mL), sodium acetate (0.072 g, 0.884 mmol) and 3-(4-fluorobenzyl)-2-thioxothiazolidin-4-one (20.1a, 0.116 g, 0.486 mmol) were added at room temperature. The reaction mixture was heated at 120° C. for 78 h. After completion, reaction mixture was cooled, water (20 mL) was added and the precipitate formed was collected by filtration. The product was purified by washings with water and diethyl ether to afford (Z)-3′-((3-(4-fluorobenzyl)-4-oxo-2-thioxothiazolidin-5-ylidene)methyl)-[1,1′-biphenyl]-4-carboxylic acid (20) as yellow solid. Yield: 0.210 g, 52.8%. MS (ES⁻) m/z=448.0 [M−1], ¹H NMR (400 MHz, d6-DMSO) δ 13.04 (bs, 1H), 8.06 (m, 3H), 7.97 (s, 1H), 7.88 (m, 3H), 7.70 (m, 2H), 7.40 (m, 2H), 7.18 (t, 2H, J=8.8 Hz), 5.24 (s, 2H)

### Example 21. Preparation of (Z)-4-(6-((3-(4-Fluorobenzyl)-4-oxo-2-thioxothiazolidin-5-ylidene)methyl)pyridin-2-yl)benzoic acid (21)

The title compound was prepared according to Scheme 21.

Synthesis of 4-(6-formylpyridin-2-yl)benzoic acid (21.1). To a solution of 4-boronobenzoic acid (21.2, 1.0 g, 6.0 mmol) and 6-bromopicolinaldehyde (21.2a, 1.12 g 6.0 mmol) in dioxane (20 mL) and ethanol (20 mL), water (5 mL) was added at room temperature. The reaction mixture was purged with argon for 15 minutes with stirring and PdCl₂ (PPh₃)₂ (0.211 g, 0.30 mmol) and sodium carbonate (1.91 g, 18.0 mmol) were added.

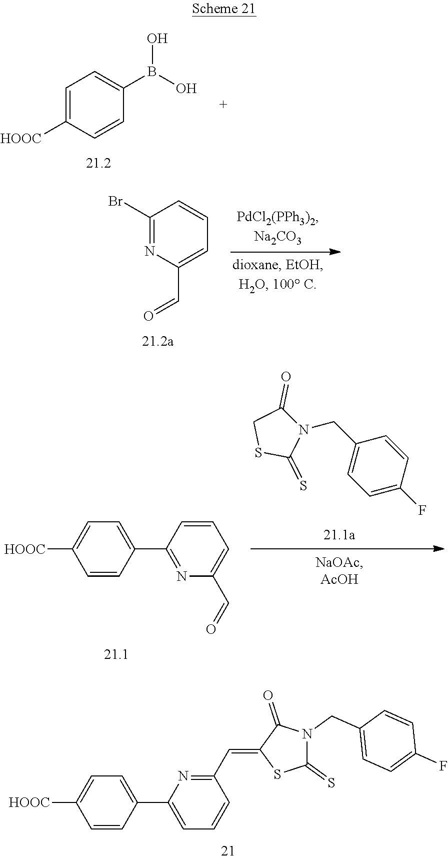 [SMILES: O=C(O)c1ccc(B(O)O)cc1 ; O=Cc1cccc(Br)n1 ; O=C1CSC(=S)N1Cc1ccc(F)cc1 ; O=Cc1cccc(-c2ccc(C(=O)O)cc2)n1 ; O=C(O)c1ccc(-c2cccc(/C=C3\SC(=S)N(Cc4ccc(F)cc4)C3=O)n2)cc1]

The mixture was again purged with argon for 10 minutes and heated the mixture at 100° C. for 6 h. Solvents were removed under reduced pressure and the crude was purified by silica gel (100-200 mesh) column chromatography eluting with 50% ethyl acetate in hexanes 4-(6-formylpyridin-2-yl)benzoic acid (21.1). Yield: 0.350 g, 25.73%.

Synthesis of (Z)-4-(6-((3-(4-fluorobenzyl)-4-oxo-2-thioxothiazolidin-5-ylidene)methyl)pyridin-2-yl)benzoic acid (21). To a solution of 4-(6-formylpyridin-2-yl)benzoic acid (21.1, 0.150 g, 6.6 mmol) in acetic acid (3 mL), sodium acetate (0.081 g, 9.9 mmol) and 3-(4-fluorobenzyl)-2-thioxothiazolidin-4-one (21.1a, 0.175 g, 7.2 mmol) were added at room temperature. The reaction mixture was heated at 110° C. for 12 h. After completion, water (15 mL) was added and the precipitate formed was collected by filtration. The product was purified by washings with water, diethyl ether and n-pentane to afford (Z)-4-(6-((3-(4-fluorobenzyl)-4-oxo-2-thioxothiazolidin-5-ylidene)methyl)pyridin-2-yl)benzoic acid (21). Yield: 0.120 g, 40.40%. MS (ES⁻) m/z=448.9 [M−1], (ES⁺) m/z=451.1 [M+1], ¹H NMR (400 MHz, d6-DMSO) δ 13.14 (bs, 1H), 8.11 (t, 6H, J=66.1 Hz), 7.28 (d, 4H, J=89.8 Hz), 5.25 (s, 2H)

### Example 22. Preparation of (Z)-4-(2-((3-(4-Fluorobenzyl)-4-oxo-2-thioxothiazolidin-5-ylidene)methyl)pyridin-4-yl)benzoic acid (22)

The title compound was prepared according to Scheme 22.

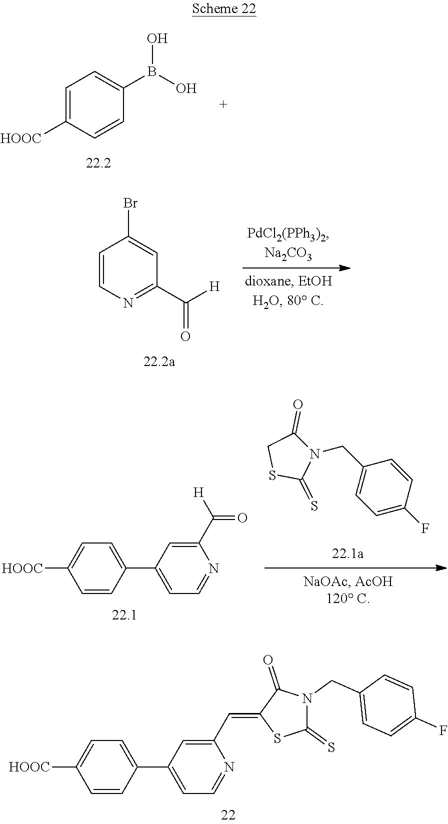 [SMILES: O=C(O)c1ccc(B(O)O)cc1 ; O=Cc1cc(Br)ccn1 ; O=Cc1cc(-c2ccc(C(=O)O)cc2)ccn1 ; O=C1CSC(=S)N1Cc1ccc(F)cc1 ; O=C(O)c1ccc(-c2ccnc(/C=C3\SC(=S)N(Cc4ccc(F)cc4)C3=O)c2)cc1]

Synthesis of 4-(2-formylpyridin-4-yl)benzoic acid (22.1). A solution of 4-boronobenzoic acid (22.2, 2.0 g, 12.0 mmol) and 4-bromopicolinaldehyde (22.2a, 2.24 g 12.0 mmol) in dioxane (30 mL), ethanol (30 mL) and a solution of sodium carbonate in water (8 mL) was purged with argon for 15 minutes followed by addition of PdCl₂ (PPh₃)₂ (0.42 g, 0.60 mmol). The mixture was again purged with argon for 10 minutes and then heated at 80° C. for 9 h. After completion, solvents were removed under reduced pressure and the mixture was diluted with ice cold water (100 mL) and extracted with ethyl acetate (450 mL). The organic layer was dried over anhydrous sodium sulfate and concentrated under reduced pressure to get crude. The crude was purified by silica gel (100-200 mesh) column chromatography eluting with 5-10% methanol in dichloromethane to afford 4-(2-formylpyridin-4-yl)benzoic acid (22.1). Yield: 0.250 g, 9.25%.

Synthesis of (Z)-4-(2-((3-(4-fluorobenzyl)-4-oxo-2-thioxothiazolidin-5-ylidene)methyl)pyridin-4-yl)benzoic acid (22). To a solution of 4-(2-formylpyridin-4-yl)benzoic acid (22.1, 0.10 g, 4.4 mmol) in acetic acid (3 mL), sodium acetate (0.054 g, 6.6 mmol) and 3-(4-fluorobenzyl)-2-thioxothiazolidin-4-one (22.1a, 0.116 g, 4.8 mmol) were added at room temperature. The reaction mixture was heated at 110° C. for 14 h. After completion, water (15 mL) was added and the precipitate formed was collected by filtration. The product was purified by washings with water, diethyl ether and n-pentane to afford, (Z)-4-(2-((3-(4-fluorobenzyl)-4-oxo-2-thioxothiazolidin-5-ylidene)methyl)pyridin-4-yl)benzoic acid (22). Yield: 0.135 g, 68.18%. MS (ES⁻) m/z=449.0 [M−1], ¹H NMR (400 MHz, d6-DMSO) δ 13.18 (bs, 1H), 8.90 (d, 1H, J=5.2 Hz), 8.42 (s, 1H), 8.04 (dd, 4H, J=43.2, 8.2 Hz) 7.95 (s, 1H), 7.85 (d, 1H, J=5.2 Hz), 7.40 (t, 2H, J=7.7 Hz), 7.18 (t, 2H, J=8.8 Hz), 5.24 (s, 2H)

### Example 23. Preparation of methyl (Z)-3′-((3-Benzyl-4-oxo-2-thioxothiazolidin-5-ylidene)methyl)[-1,1′-biphenyl]-4-carboxylate (23)

The title compound was prepared according to Scheme 23.

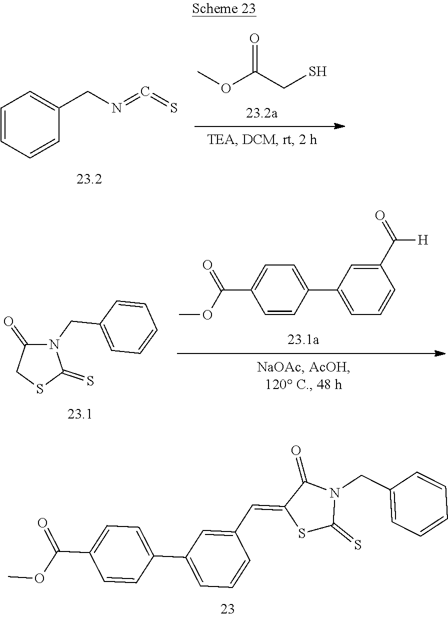

Synthesis of 3-benzyl-2-thioxothiazolidin-4-one (23.1). To a solution of isothiocyanato methyl benzene (23.2, 5.0 g, 33.5 mmol) in dichloromethane (100 mL) was added triethylamine (4.7 mL, 33.5 mmol) and methyl 2-mercaptoacetate (23.2a, 3.55 g, 33.5 mmol) at 0° C. and the reaction was brought to room temperature and stirred for 2 h. After completion, water (100 mL) was added and mixture was extracted with dichloromethane. The organic layer was separated, dried over anhydrous sodium sulfate and concentrated under reduced pressure to get the crude product. The crude product was purified by washing with diethyl ether and pentane to afford 3-benzyl-2-thioxothiazolidin-4-one (23.1) as red solid. Yield: 4.80 g, 64.4%.

Synthesis of methyl (Z)-3′-((3-benzyl-4-oxo-2-thioxothiazolidin-5-ylidene)methyl)-[1,1′-biphenyl]-4-carboxylate (23). To a solution of methyl 3′-formyl-[1,1′-biphenyl]-4-carboxylate (23.1, 0.30 g, 1.25 mmol) in acetic acid (10 mL) was added sodium acetate (0.153 g, 1.87 mmol) and 3-benzyl-2-thioxothiazolidin-4-one (23.1a, 0.278 g, 1.25 mmol) at room temperature. The reaction mixture was heated at 120° C. for 3 h. After completion, water (30 mL) was added and a precipitate formed which was collected by filtration. The product was purified by washing with water, diethyl ether and methanol to afford (Z)-3′-((3-benzyl-4-oxo-2-thioxothiazolidin-5-ylidene)methyl)-[1,1′-biphenyl]-4-carboxylate (23) as brown solid. Yield: 0.25 g, 44.9%. MS (ES⁺) m/z=446.2 [M+1], ¹H NMR (400 MHz, d6-DMSO) δ 8.08 (bs, 1H), 8.00 (dd, 4H, J=72.4, 8.2 Hz), 7.98 (s, 1H), 7.90 (m, 1H), 7.70 (m, 2H), 7.32 (m, 5H), 5.27 (s, 2H), 3.89 (s, 3H).

### Example 24. Preparation of Methyl (Z)-4-(6-((3-benzyl-4-oxo-2-thioxothiazolidin-5-ylidene)methyl)pyridin-2-yl)benzoate (24)

The title compound was prepared according to Scheme 24.

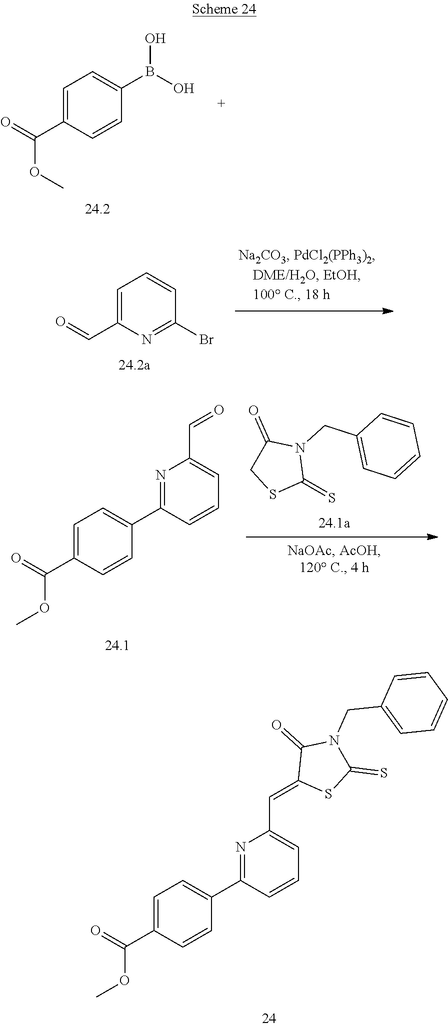

Synthesis of methyl 4-(6-formylpyridin-2-yl)benzoate (24.1). To a solution of (4-(methoxycarbonyl)phenyl)boronic acid (24.2, 1.0 g, 5.55 mmol) in ethanol (10 mL) and DME (10 mL), 6-bromopicolinaldehyde (24.2a, 1.03 g, 5.55 mmol) and Na₂CO₃ (1.50 g, 13.8 mmol) in water (5.0 mL) were added at room temperature. The reaction mixture was degassed with argon for 15 min and PdCl₂ (PPh₃)₂ (0.10 eq) was added followed by heating the mixture at 100° C. temperature for 18 h. Cooled reaction mixture at room temperature, removed solvents under reduced pressure and water (100 mL) was added. The product was extracted in ethyl acetate and organic layer was separated, dried over anhydrous sodium sulfate and concentrated under reduced pressure to get crude. The crude product was purified by silica gel (230-400 mesh) column chromatography eluting with 10% ethyl acetate in hexane to afford methyl 4-(6-formylpyridin-2-yl)benzoate (24.1) as yellow color solid. Yield: 0.650 g, 48.8%.

Synthesis of methyl (Z)-4-(6-((3-benzyl-4-oxo-2-thioxothiazolidin-5-ylidene)methyl)pyridin-2-yl)benzoate (24). To a solution of methyl 4-(6-formylpyridin-2-yl)benzoate (24.1, 0.20 g, 0.829 mmol) in acetic acid (15 mL), sodium acetate (0.102 g, 1.24 mmol) and 3-benzyl-2-thioxothiazolidin-4-one (24.1a, 0.203 g, 0.912 mmol) were added at room temperature. The reaction mixture was heated at 120° C. for 4 h. After completion, water (30 mL) was added and precipitate formed was collected by filtration. The product was purified by washings with diethyl ether and pentane to afford (Z)-4-(6-((3-benzyl-4-oxo-2-thioxothiazolidin-5-ylidene)methyl)pyridin-2-yl)benzoic acid (24) as brown solid. Yield: 0.260 g, 70.0%. MS (ES⁺) m/z=447.2 [M+1], ¹H NMR (400 MHz, d6-DMSO) δ 8.24 (dd, 4H, J=64.5, 8.3 Hz), 8.12 (d, 2H, J=4.1 Hz), 8.00 (t, 1H, J=4.1 Hz), 7.97 (s, 1H), 7.33 (m, 5H), 5.27 (s, 2H), 3.91 (s, 3H)

### Example 25. Preparation of (Z)-4-(5-((3-Benzyl-4-oxo-2-thioxothiazolidin-5-ylidene)methyl)thiophen-2-yl)benzoic acid (25)

The title compound was prepared according to Scheme 25.

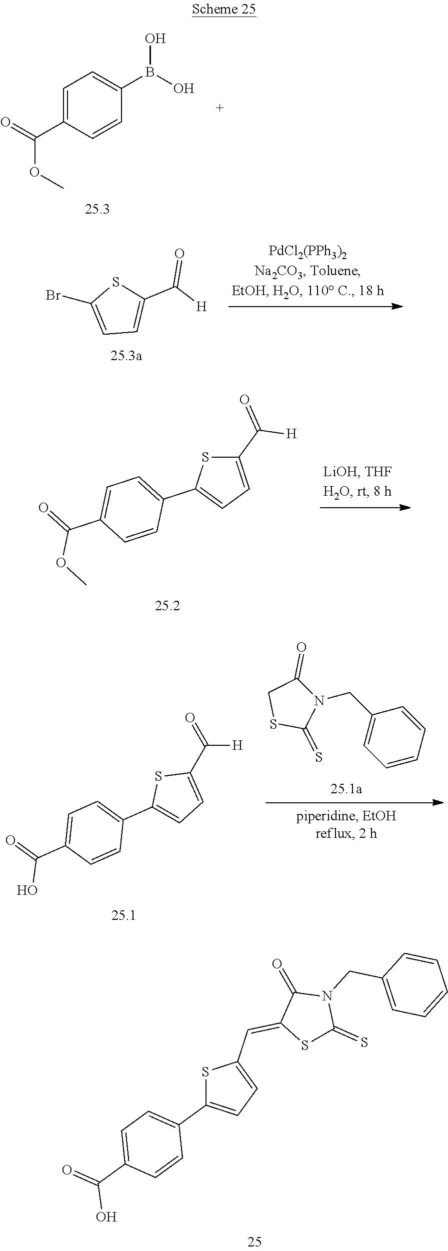

Synthesis of methyl 4-(5-formylthiophen-2-yl)benzoate (25.2). To a solution of (4-(methoxycarbonyl)phenyl)boronic acid (25.3, 4.0 g, 22.0 mmol) and 5-bromothiophene-2-carbaldehyde (25.3a, 4.24 g 22.0 mmol) in toluene (40 mL) and ethanol (40 mL), water (10 mL) was added at room temperature. The reaction mixture was purged with argon for 15 minutes and PdCl₂(PPh₃)₂ (0.776 g, 1.1 mmol), sodium carbonate (5.88 g, 55.5 mmol) were added at room temperature. The reaction mixture was again purged with argon for 10 minutes and heated at 110° C. for 18 h. After completion, reaction mass was cooled to room temperature and solvents were removed under reduced pressure. The crude was diluted with ice-cold water (100 mL) and extracted with dichloromethane (2×200 mL). The organic layer was separated, dried over anhydrous sodium sulfate and concentrated under reduced pressure. The crude product was purified by silica gel (100-200 mesh) column chromatography eluting with 20-30% ethyl acetate in hexanes to afford methyl 4-(5-formylthiophen-2-yl)benzoate (25.2), Yield: 2.50 g, 45.78%.

Synthesis of 4-(5-formylthiophen-2-yl)benzoic acid (25.1). To a solution of methyl 4-(5-formylthiophen-2-yl)benzoate (25.2, 1.50 g, 6.0 mmol) in THF (15 mL), LiOH (0.439 g, 18.2 mmol) dissolved in water (2.0 mL) was added at room temperature and stirred for 8 h. After completion, THF was removed under reduced pressure, water (30 mL) was added and pH was adjusted to ˜7 by addition of citric acid solution. The precipitated solid was collected by filtration and the product was purified by washings with diethyl ether and pentane to afford 4-(5-formylthiophen-2-yl)benzoic acid (25.1) as yellow solid.Yield: 1.10 g, 78.0%.

Synthesis of (Z)-4-(5-((3-benzyl-4-oxo-2-thioxothiazolidin-5-ylidene)methyl)thiophen-2-yl)benzoic acid (25). To a solution of 4-(5-formylthiophen-2-yl)benzoic acid (25.1, 1.0 g, 4.3 mmol) in ethanol (20 mL), 3-benzyl-2-thioxothiazolidin-4-one (25.1a, 0.96 g, 4.3 mmol) and piperidine (0.36 g, 4.3 mmol) were added at room temperature. The reaction mixture was then heated at 90° C. for 2 h. After completion, ethanol was removed under reduced pressure, water (30 mL) was added and pH was adjusted to ˜7 by addition of citric acid solution. The precipitated solid was collected by filtration and the product was purified by washings with diethyl ether and pentane to afford (Z)-4-(5-((3-benzyl-4-oxo-2-thioxothiazolidin-5-ylidene)methyl)thiophen-2-yl)benzoic acid (25) as brown solid.Yield: 0.90 g, 50.0%. MS (ES⁻) m/z=436.4 [M−1], ¹H NMR (400 MHz, d6-DMSO) δ 13.12 (bs, 1H), 8.15 (s, 1H), 7.97 (dd, 4H, J=26.7, 8.1 Hz), 7.83 (dd, 2H, J=22.0, 3.6 Hz), 7.33 (m, 5H), 5.24 (s, 2H)

### Example 26. Preparation of (Z)-4-(5-((3-(2-Methylbenzyl)-4-oxo-2-thioxothiazolidin-5-ylidene)methyl)furan-2-yl)benzoic acid (26)

The title compound was prepared according to Scheme 26.

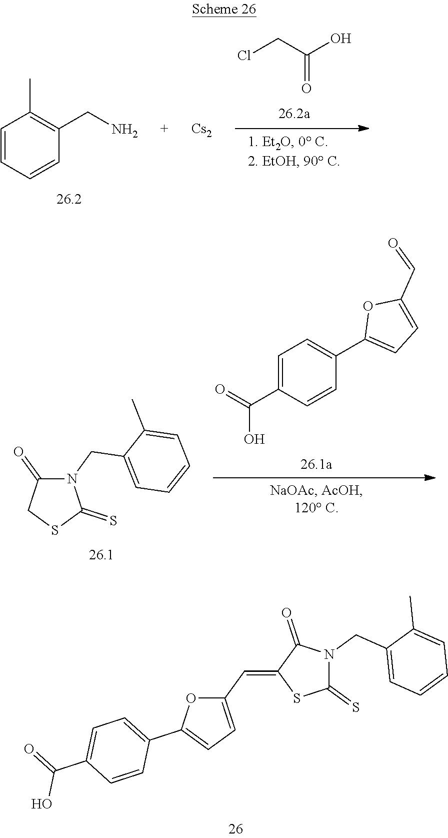

Synthesis of 3-(2-methylbenzyl)-2-thioxothiazolidin-4-one (26.1). A solution of o-tolylmethanamine (26.2, 1.0 g, 8.2 mmol) in diethyl ether (15 mL) under nitrogen was treated with carbon disulfide (0.62 g, 8.2 mmol) at 0° C. The suspension was stirred at 0° C. for 2 h and the precipitate formed was collected by filtration, washed with diethyl ether and dried under vacuum. This solid was dissolved in ethanol (15 mL) and chloroacetic acid (26.2a, 0.77 g, 8.2 mmol) was added in one portion. The reaction mixture was heated at 90° C. for 16 h. The mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(2-methylbenzyl)-2-thioxothiazolidin-4-one (26.1) as a brown solid. Yield: 0.220 g, 11.28%.

Synthesis of (Z)-4-(5-((3-(2-methylbenzyl)-4-oxo-2-thioxothiazolidin-5-ylidene)methylguran-2-yl)benzoic acid (26). To a solution of 3-(2-methylbenzyl)-2-thioxothiazolidin-4-one (26.1, 0.20 g, 0.84 mmol) in acetic acid (5 mL) was added sodium acetate (0.180 g, 0.84 mmol) and 4-(5-formylfuran-2-yl)benzoic acid (26.1a, 0.180 g, 0.84 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5-((3-(2-methylbenzyl)-4-oxo-2-thioxothiazolidin-5-ylidene)methyl)furan-2-yl)benzoic acid (26). Yield: 0.180 g, 49.04%. MS (ES⁻) m/z=434.1 [M−1], ¹H NMR (400 MHz, d6-DMSO) δ 13.11 (bs, 1H), 8.05 (dd, 4H, J=49.0, 8.4 Hz), 7.78 (s, 1H), 7.48 (dd, 1H, J=29.9, 3.7 Hz), 7.22 (d, 1H, J=7.2 Hz), 7.17 (t, 1H, J=7.2 Hz), 7.11 (t, 1H, J=7.2 Hz), 6.81 (d, 1H, J=7.2 Hz) 5.21 (s, 2H), 2.40 (s, 3H)

### Example 27. Preparation of (Z)-4-(5-((3-(2-Methoxybenzyl)-4-oxo-2-thioxothiazolidin-5-ylidene)methyl)furan-2-yl)benzoic acid (27)

The title compound was prepared according to Scheme 27.

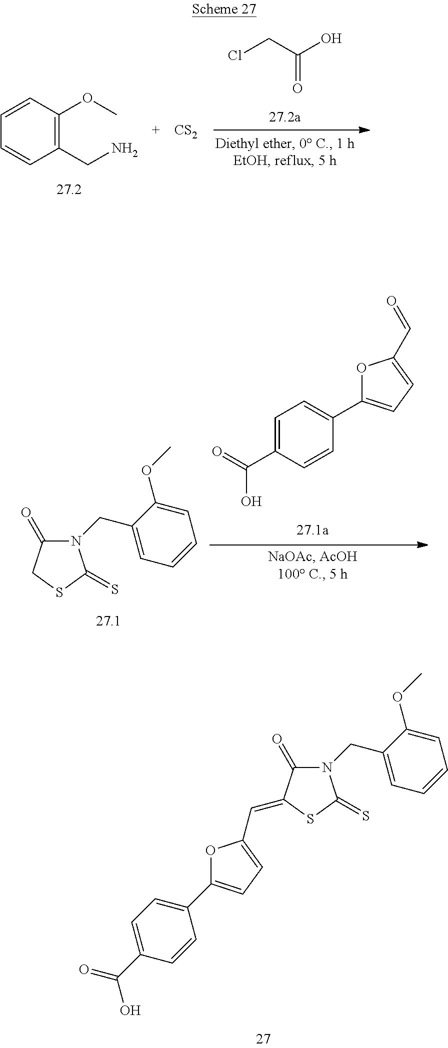

Synthesis of 3-(2-methoxybenzyl)-2-thioxothiazolidin-4-one (27.1). A solution of (2-methoxyphenyl)methanamine (27.2, 1.5 g, 10.9 mmol) in diethyl ether (30 mL) under nitrogen was treated with carbon disulfide (1.24 g, 16.3 mmol) at 0° C. The suspension was stirred at 0° C. for 1 h and the solid formed was collected by filtration, washed with diethyl ether and dried under vacuum. The obtained solid was dissolved in ethanol (20 mL) and chloroacetic acid (27.2a, 1.56 g, 16.5 mmol) was added in one portion. The reaction mixture was heated at 100° C. for 5 h. After completion, the mixture was diluted with ice cold water (50 mL) and extracted with ethyl acetate (2×50 mL). The organic layer was dried over anhydrous sodium sulfate and concentrated under reduced pressure to get crude. The crude was purified by silica gel (100-200 mesh) column chromatography eluting with 5-10% ethyl acetate in hexane to afford 3-(2-methoxybenzyl)-2-thioxothiazolidin-4-one (27.1) as a yellow solid. Yield: 0.610 g, 25.0%.

Synthesis of (Z)-4-(5-((3-(2-methoxybenzyl)-4-oxo-2-thioxothiazolidin-5-ylidene)methyl)furan-2-yl)benzoic acid (27). To a solution of 3-(2-methoxybenzyl)-2-thioxothiazolidin-4-one (27.1, 0.20 g, 0.79 mmol) in acetic acid (10 mL), sodium acetate (0.098 g, 1.20 mmol) and 4-(5-formylfuran-2-yl)benzoic acid (27.1a, 0.171 g, 0.79 mmol) were added at room temperature. The reaction mixture was heated at 120° C. for 5 h. After completion, reaction mixture was cooled, water (20 mL) was added and the precipitate formed was collected by filtration. The product was purified by washings with water, diethyl ether and methanol to afford (Z)-4-(5-((3-(2-methoxybenzyl)-4-oxo-2-thioxothiazolidin-5-ylidene)methyl)furan-2-yl)benzoic acid (27) as brown solid. Yield: 0.155 g, 44.3.0%. MS (ES⁻) m/z=450.1 [M−1], ¹H NMR (400 MHz, d6-DMSO) δ 13.11 (bs, 1H), 8.04 (dd, 4H, J=51.1, 8.4 Hz), 7.75 (s, 1H), 7.47 (dd, 2H, J=32.2, 3.7 Hz), 7.26 (t, 1H, J=6.8 Hz), 7.03 (d, 1H, J=8.2 Hz), 6.86 (t, 1H, J=7.4 Hz), 6.80 (d, 1H, J=7.1 Hz), 5.18 (s, 2H), 3.85 (s, 3H)

### Example 28. Preparation of (Z)-4-(5-((3-(3-Methylbenzyl)-4-oxo-2-thioxothiazolidin-5-lidene)methyl)furan-2-yl)benzoic acid (28)

The title compound was prepared according to Scheme 28.

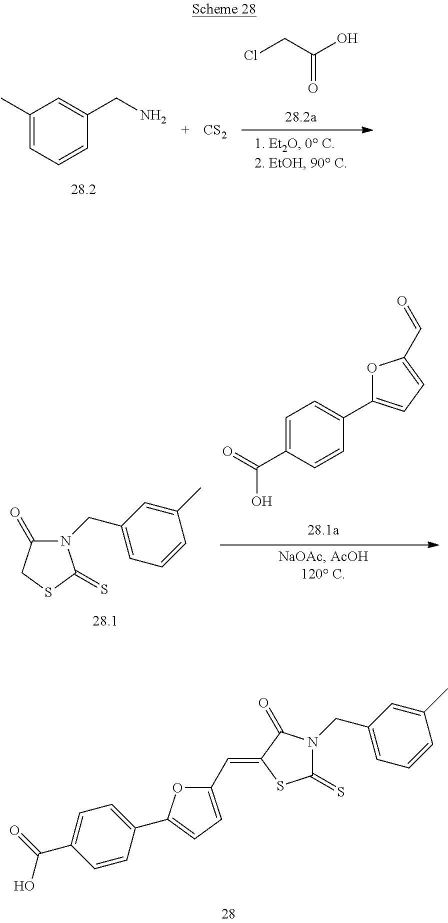

Synthesis of 3-(3-methylbenzyl)-2-thioxothiazolidin-4-one (28.1). A solution of m-tolylmethanamine (28.2, 1.0 g, 8.25 mmol) in diethyl ether (15 mL) under nitrogen was treated with carbon disulfide (0.62 g, 8.25 mmol) at 0° C. The suspension was stirred at 0° C. for 2 h and the precipitate formed was collected by filtration, washed with diethyl ether and dried under vacuum. This solid was dissolved in ethanol (15 mL) and chloroacetic acid (28.2a, 1.16 g, 12.0 mmol) was added in one portion. The reaction mixture was heated at 90° C. for 16 h. The mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(3-methylbenzyl)-2-thioxothiazolidin-4-one (28.1) as an off white solid. Yield: 0.250 g, 12.82%.

Synthesis of (Z)-4-(5-((3-(3-methylbenzyl)-4-oxo-2-thioxothiazolidin-5-ylidene)methylguran-2-yl)benzoic acid (28). To a solution of 3-(3-methylbenzyl)-2-thioxothiazolidin-4-one (28.1, 0.250 g, 1.0 mmol) in acetic acid (3 mL) was added sodium acetate (0.129 g, 1.5 mmol) and 4-(5-formylfuran-2-yl) benzoic acid (28.1a, 0.228 g, 1.0 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5 -((3 -(3 -methylbenzyl)-4-oxo-2-thioxothiazolidin-5 -ylidene)methyl)furan-2-yl)benzoic acid (28).Yield: 0.060 g, 13.07%. MS (ES⁻) m/z=434.0 [M−1], ¹H NMR (400 MHz, d6-DMSO) δ 13.12 (bs, 1H), 8.04 (dd, 4H, J=51.2, 7.2 Hz), 7.75 (s, 1H), 7.51 (bs, 1H), 7.43 (bs, 1H), 7.22 (m, 1H), 7.11 (m, 3H), 5.21 (s, 2H), 2.27 (s, 3H)

### Example 29. Preparation of (Z)-4-(5-((3-(3-Methoxybenzyl)-4-oxo-2-thioxothiazolidin-5-ylidene)methyl)furan-2-yl)benzoic acid (29)

The title compound was prepared according to Scheme 29.

Synthesis of 3-(3-methoxybenzyl)-2-thioxothiazolidin-4-one (29.1). A solution of (3-methoxyphenyl)methanamine (29.2, 1.5 g, 10.9 mmol) in diethyl ether (30 mL) under nitrogen was treated with carbon disulfide (1.24 g, 16.3 mmol) at 0° C. The suspension was stirred at 0° C. for 1 h and the solid formed was collected by filtration, washed with diethyl ether and dried under vacuum. This solid obtained was dissolved in ethanol (20 mL) and chloroacetic acid (29.2a, 1.54 g, 16.3 mmol) was added in one portion at room temperature.

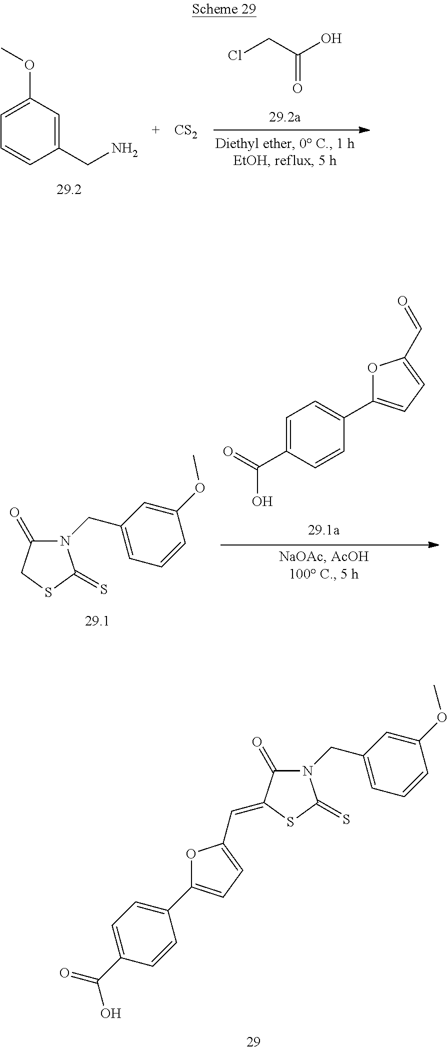

The reaction mixture was heated at 100° C. for 5 h. After completion, the reaction mixture was cooled, diluted with ice cold water (20 mL) and extracted with ethyl acetate (2×50 mL). The organic layer was dried over anhydrous sodium sulfate and concentrated under reduced pressure. The crude product was purified by silica gel (100-200 mesh) column chromatography eluting with 5-10% ethyl acetate in hexanes to afford 3-(3-methoxybenzyl)-2-thioxothiazolidin-4-one (29.1) as a yellow solid. Yield: 0.55 g, 23.0%.

Synthesis of (Z)-4-(5-((3-(3-methoxybenzyl)-4-oxo-2-thioxothiazolidin-5-ylidene)methyl)furan-2-yl)benzoic acid (29). To a solution of 3-(3-methoxybenzyl)-2-thioxothiazolidin-4-one (29.1, 0.20 g, 0.79 mmol) in acetic acid (10 mL), sodium acetate (0.098 g, 1.20 mmol) and 4-(5-formylfuran-2-yl)benzoic acid (29.1a, 0.171 g, 0.79 mmol) were added at room temperature. The reaction mixture was heated at 120° C. for 5 h. After completion, reaction mixture was cooled, water (20 mL) was added and the precipitate formed was collected by filtration. The product was purified by washings with water, diethyl ether and methanol to afford (Z)-4-(5-((3-(3-methoxybenzyl)-4-oxo-2-thioxothiazolidin-5-ylidene)methyl)furan-2-yl)benzoic acid (29) as brown solid. Yield: 0.120 g, 34.0%. MS (ES⁻) m/z=450.0 [M−1], ¹H NMR (400 MHz, d6-DMSO) δ 13.11 (bs, 1H), 8.04 (dd, 4H, J=52.3, 8.2 Hz), 7.75 (s, 1H), 7.46 (dd, 2H, J=30.4, 3.6 Hz), 7.25 (t, 1H, J=7.7 Hz), 6.87 (m, 3H), 5.22 (s, 2H), 3.73 (s, 3H)

### Example 30. Preparation of (Z)-4-(5-((3-(3-Chlorobenzyl)-4-oxo-2-thioxothiazolidin-5-ylidene)methyl)furan-2-yl)benzoic acid (30)

The title compound was prepared according to Scheme 30.

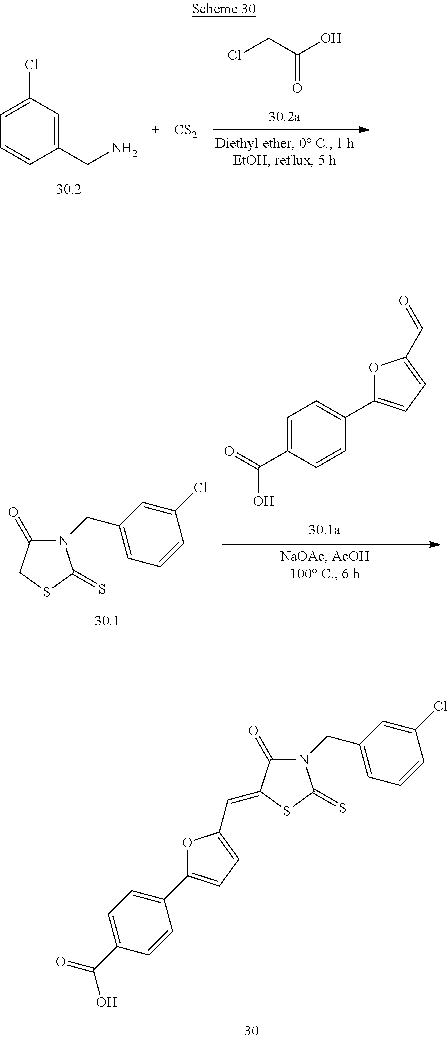

Synthesis of 3-(3-chlorobenzyl)-2-thioxothiazolidin-4-one (30.1). A solution of (3-chlorophenyl)methanamine (30.2, 1.50 g, 10.5 mmol) in diethyl ether (30 mL) under nitrogen was treated with carbon disulfide (0.80 g, 10.5 mmol) at 0° C. The suspension was stirred at 0° C. for 1 h and the solid formed was collected by filtration, washed with diethyl ether and dried under vacuum. The solid product was dissolved in ethanol (20 mL) and chloroacetic acid (30.2a, 1.0 g, 10.5 mmol) was added in one portion at room temperature. The reaction mixture was heated at 100° C. for 4 h. After completion, the mixture was diluted with ice cold water (20 mL) and extracted with ethyl acetate (2×50 mL). The organic layer was dried over anhydrous sodium sulfate and concentrated under reduced pressure. The crude product was purified by silica gel (100-200 mesh) column chromatography eluting with 5-10% ethyl acetate in hexanes to afford 3-(3-chlorobenzyl)-2-thioxothiazolidin-4-one (30.1) as a yellow solid. Yield: 0.80 g, 27.0%.

Synthesis of (Z)-4-(5-((3-(3-chlorobenzyl)-4-oxo-2-thioxothiazolidin-5-ylidene)methylguran-2-yl)benzoic acid (30). To a solution of 3-(3-chlorobenzyl)-2-thioxothiazolidin-4-one (30.1, 0.20 g, 0.77 mmol) in acetic acid (10 mL), sodium acetate (0.120 g, 0.77 mmol) and 4-(5-formylfuran-2-yl)benzoic acid (30.1a, 0.168 g, 0.77 mmol) were added at room temperature. The reaction mixture was heated at 120° C. for 6 h. After completion, reaction mixture was cooled, water (20 mL) was added and the precipitate formed was collected by filtration. The product was purified by washings with water, diethyl ether and methanol to afford (Z)-4-(5-((3-(3-chlorobenzyl)-4-oxo-2-thioxothiazolidin-5-ylidene)methyl)furan-2-yl)benzoic acid (30) as brown solid. Yield: 0.19 g, 54.0%. MS (ES⁻) m/z=454.0 [M-1], ¹H NMR (400 MHz, d6-DMSO) δ 13.10 (bs, 1H), 8.04 (dd, 4H, J=51.6, 8.1 Hz), 7.76 (s, 1H), 7.47 (dd, 2H, J=29.0, 3.4 Hz), 7.38 (m, 3H), 7.28 (m, 1H), 5.26 (s, 2H)

### Example 31. Preparation of (Z)-4-(5-((3-(2,3-Dimethylbenzyl)-4-oxo-2-thioxothiazolidin-5-ylidene)methyl)furan-2-yl)benzoic acid (31)

The title compound was prepared according to Scheme 31.

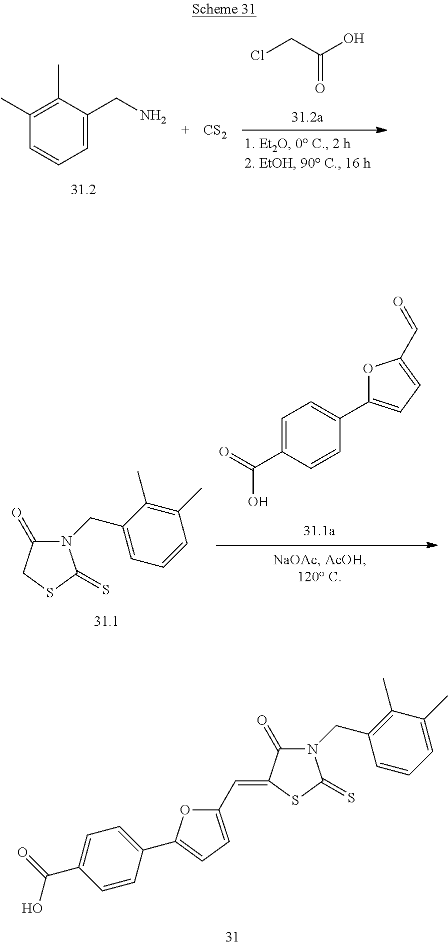

Synthesis of 3-(2,3-dimethylbenzyl)-2-thioxothiazolidin-4-one (31.1). A solution of (2,3-dimethylphenyl)methanamine (31.2, 1.0 g, 7.40 mmol) in diethyl ether (15 mL) under nitrogen was treated with carbon disulfide (0.56 g, 7.40 mmol) at 0° C. The suspension was stirred at 0° C. for 2 h and the precipitate formed was collected by filtration, washed with diethyl ether and dried under vacuum. This solid was dissolved in ethanol (15 mL) and chloroacetic acid (31.2a, 1.04 g, 11.0 mmol) was added in one portion. The reaction mixture was heated at 90° C. for 16 h. After completion, the mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(2,3-dimethylbenzyl)-2-thioxothiazolidin-4-one (31.1) as a brown solid. Yield: 0.21 g, 11.66%.

Synthesis of (Z)-4-(5-((3-(2,3-dimethylbenzyl)-4-oxo-2-thioxothiazolidin-5-ylidene)methyl)furan-2-yl)benzoic acid (31). To a solution of 3-(2,3-dimethylbenzyl)-2-thioxothiazolidin-4-one (31.1, 0.170 g, 0.67 mmol) in acetic acid (4 mL) was added sodium acetate (0.083 g, 0.99 mmol) and 4-(5-formylfuran-2-yl)benzoic acid (31.1a, 0.131 g, 0.60 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5-((3-(2,3-dimethylbenzyl)-4-oxo-2-thioxothiazolidin-5-ylidene)methyl)furan-2-yl)benzoic acid (31). Yield: 0.110 g, 31%. MS (ES⁻) m/z=448.0 [M−1], ¹H NMR (400 MHz, d6-DMSO) δ 13.11 (bs, 1H), 8.07 (dd, 4H, J=29.2, 7.8 Hz), 7.62 (s, 1H), 7.47 (dd, 2H, J=30.8, 2.8 Hz), 7.07 (d, 1H, J=7.24 Hz), 6.99 (t, 1H, J=7.24 Hz), 6.59 (d, 1H, J=7.24 Hz), 5.22 (s, 2H), 2.27 (s, 6H)

### Example 32. Preparation of (Z)-4-(5-((3-(4-Fluorobenzyl)-4-oxo-2-thioxothiazolidin-5-ylidene)methyl)furan-2-yl)benzoic acid (32)

The title compound was prepared according to Scheme 32.

Synthesis of 3-(4-fluorobenzyl)-2-thioxothiazolidin-4-one (32.1). A solution of (4-fluorophenyl)methanamine (32.2, 1.0 g, 8.0 mmol) in diethyl ether (10 mL) under nitrogen was treated with carbon disulfide (0.61 g, 8.0 mmol) at 0° C. The suspension was stirred at 0° C. for 1 h and the solid formed was collected by filtration, washed with diethyl ether and dried under vacuum. The solid obtained was dissolved in ethanol (20 mL) and chloroacetic acid (32.2a, 0.38 g, 4.0 mmol) was added in one portion. The reaction mixture was heated at 100° C. for 5 h. After completion, the reaction mixture was diluted with ice-cold water (20 mL) and extracted with ethyl acetate (50 mL). The organic layer was dried over anhydrous sodium sulfate and concentrated under reduced pressure to get crude. The crude was purified by silica gel (100-200 mesh) column chromatography eluting with 5-10% ethyl acetate in

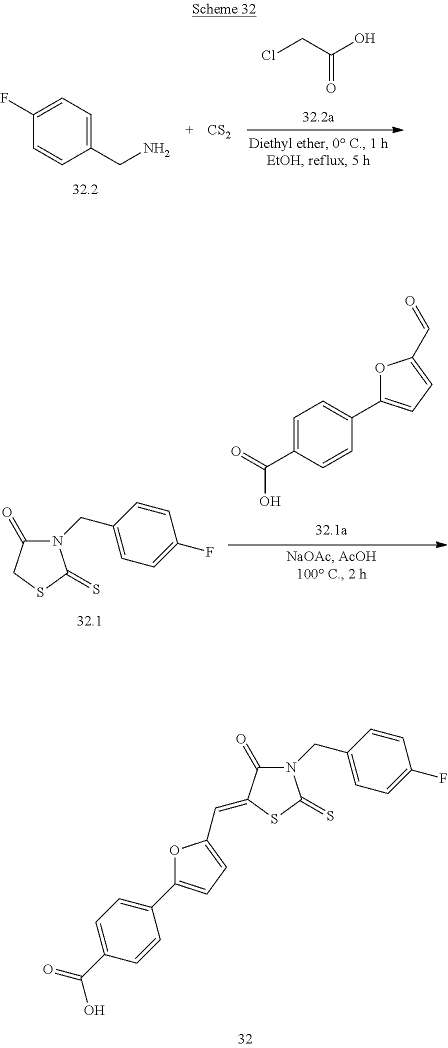

hexanes to afford 3-(4-fluorobenzyl)-2-thioxothiazolidin-4-one (32.1) as a yellow solid. Yield: 0.40 g, 21.0%.

Synthesis of (Z)-4-(5-((3-(4-chlorobenzyl)-4-oxo-2-thioxothiazolidin-5-ylidene)methylguran-2-yl)benzoic acid (32). To a solution of 3-(4-fluorobenzyl)-2-thioxothiazolidin-4-one (32.1, 0.200 g, 0.82 mmol) in acetic acid (10 mL), sodium acetate (0.067 g, 0.82 mmol) and 4-(5-formylfuran-2-yl)benzoic acid (32.1a, 0.18 g, 0.82 mmol) were added at room temperature. The reaction mixture was heated at 120° C. for 2 h. After completion, reaction mixture was cooled to room temperature, water (20 mL) was added and the precipitate formed was collected by filtration. The product was purified by washings with water, diethyl ether and methanol to afford (Z)-4-(5-((3-(4-chlorobenzyl)-4-oxo-2-thioxothiazolidin-5-ylidene)methyl)furan-2-yl)benzoic acid (32) as brown solid. Yield: 0.097 g, 26.0%. MS (ES⁻) m/z=438.1 [M−1], ¹H NMR (400 MHz, d6-DMSO) δ 13.13 (bs, 1H), 8.03 (dd, 4H, J=54.0, 8.4 Hz), 7.74 (s, 1H), 7.46 (dd, 2H, J=28.6, 3.7 Hz), 7.28 (ddd, 4H, J=87.9, 8.4, 5.5 Hz), 5.23 (s, 2H)

### Example 33. Preparation of (Z)-4-(5-((3-(3,4-Dichlorobenzyl)-4-oxo-2-thioxothiazolidin-5-ylidene)methyl)furan-2-yl)benzoic acid (33)

The title compound was synthesized according to Scheme 33.

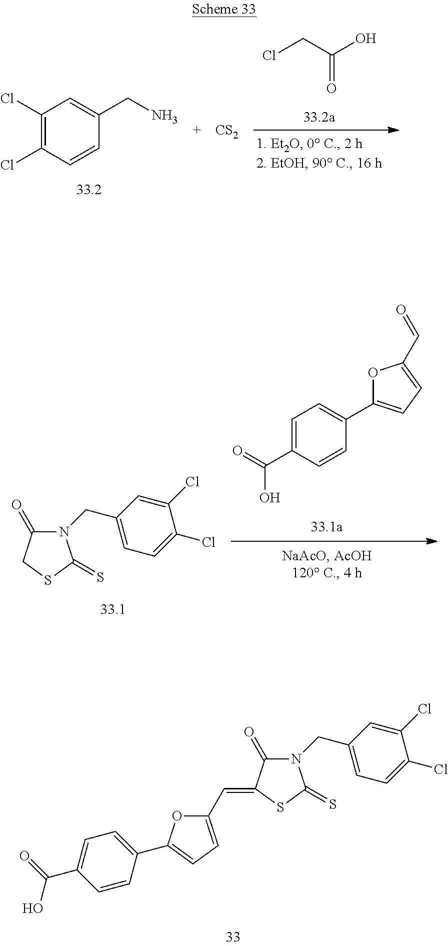

Synthesis of 3-(3,4-dichlorobenzyl)-2-thioxothiazolidin-4-one (33.1). A solution of (3,4-dichlorophenyl) methanamine (33.2, 5.0 g, 28.4 mmol) in diethyl ether (50 mL) under nitrogen was treated with carbon disulfide (1.7 mL, 28.4 mmol) at 0° C. The suspension was stirred at 0° C. for 1 h and the solid formed was collected by filtration, washed with diethyl ether and dried under vacuum. This solid was dissolved in ethanol (50 mL) and chloroacetic acid (33.2a, 2.68 g, 14.0 mmol) was added in one portion. The reaction mixture was heated at 90° C. for 6 h. After completion, the mixture was diluted with ice-cold water (100 mL) and extracted with ethyl acetate (250 mL). The organic layer was dried over anhydrous sodium sulfate and concentrated under reduced pressure to get the crude product. The crude product was purified by silica gel (100-200 mesh) column chromatography eluting with 20% ethyl acetate in hexanes and further washed with pentane to afford (3,4-dichlorobenzyl)-2-thioxothiazolidin-4-one (33.1) as a yellow solid. Yield: 1.50 g, 18%; MS (ESI) m/z=[M−1]⁻.

Synthesis of (Z)-4-(5-((3-(3,4-dichlorobenzyl)-4-oxo-2-thioxothiazolidin-5-ylidene)methyl)furan-2-yl)benzoic acid (33). To a solution of (3,4-dichlorobenzyl)-2-thioxothiazolidin-4-one (33.1, 0.50 g, 1.71 mmol) in acetic acid (5 mL) was added sodium acetate (0.211 g, 2.57 mmol) and 4-(5-formylfuran-2-yl)benzoic acid (33.1a, 0.371 g, 1.71 mmol) at room temperature. The reaction mixture was heated at 120° C. for 4 h. After completion, water (15 mL) was added and the precipitate formed was collected by filtration. The product was purified by washings with water, diethyl ether and methanol to afford (Z)-4-(5-((3-(3,4-dichlorobenzyl)-4-oxo-2-thioxothiazolidin-5-ylidene)methyl)furan-2-yl)benzoic acid (33) as a yellow solid.Yield: 0.32 g, 38%. MS (ES⁻) m/z=454.1 [M−1], ¹H NMR (400 MHz, d6-DMSO) δ 13.11 (bs, 1H), 8.05 (dd, 4H, J=49.2, 8.3 Hz), 7.79 (s, 1H), 7.52 (s, 1H), 7.48 (dd, 2H, J=23.6, 3.7 Hz), 7.31 (dt, 2H, J=9.16, 7.3 Hz), 7.01 (d, 1H, J=7.3 Hz), 5.28 (s, 2H)

### Example 34. Preparation of (Z)-4-(5-((3-(4-Methoxybenzyl)-4-oxo-2-thioxothiazolidin-5-ylidene)methyl)furan-2-yl)benzoic acid (34)

The title compound was prepared according to Scheme 34.

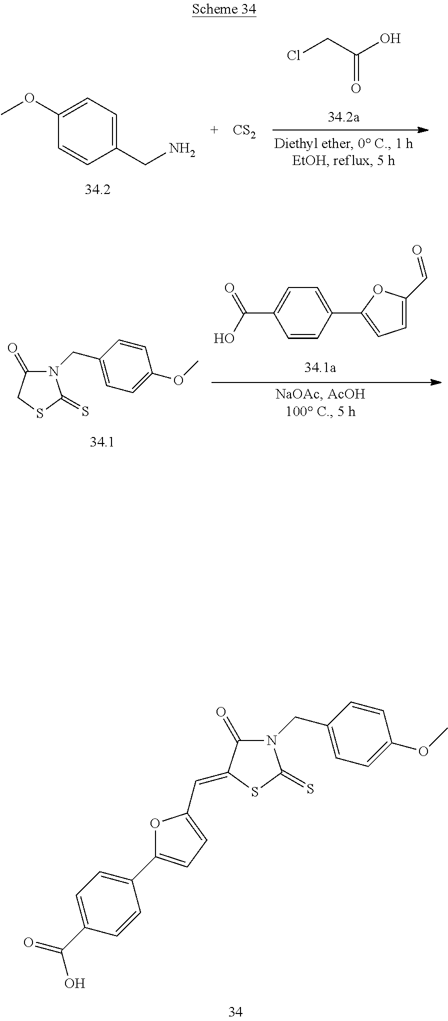

Synthesis of 3-(4-methoxybenzyl)-2-thioxothiazolidin-4-one (34.1). A solution of (4-methoxyphenyl)methanamine (34.2, 1.0 g, 7.3 mmol) in diethyl ether (20 mL) under nitrogen was treated with carbon disulfide (0.56 g, 7.3 mmol) at 0° C. The suspension was stirred at 0° C. for 1 h and the solid formed was collected by filtration, washed with diethyl ether and dried under vacuum. The solid obtained was dissolved in ethanol (20 mL) and chloroacetic acid (34.2a, 0.345 g, 3.60 mmol) was added in one portion at room temperature. The reaction mixture was heated at 100° C. for 2 h. After completion, the reaction mixture was diluted with ice cold water (20 mL) and extracted with ethyl acetate (50 mL). The organic layer was dried over anhydrous sodium sulfate and concentrated under reduced pressure. The crude was purified by silica gel (100-200 mesh) column chromatography eluting with 5-10% ethyl acetate in hexanes to afford 3-(4-methoxybenzyl)-2-thioxothiazolidin-4-one (34.1) as a yellow solid. Yield: 0.250 g, 15.0%.

Synthesis of (Z)-4-(5-((3-(4-methoxybenzyl)-4-oxo-2-thioxothiazolidin-5-ylidene)methyl)furan-2-yl)benzoic acid (34). To a solution of 3-(4-methoxybenzyl)-2-thioxothiazolidin-4-one (34.1, 0.20 g, 0.79 mmol) in acetic acid (10 mL), sodium acetate (0.064 g, 0.79 mmol) and 4-(5-formylfuran-2-yl)benzoic acid (34.1a, 0.17 g, 0.79 mmol) were added at room temperature. The reaction mixture was heated at 120° C. for 5 h. After completion, the reaction mixture was cooled to room temperature, water (20 mL) was added and the precipitate formed was collected by filtration. The product was purified by washings with water, diethyl ether and methanol to afford (Z)-4-(5-((3-(4-methoxybenzyl)-4-oxo-2-thioxothiazolidin-5-ylidene)methyl)furan-2-yl)benzoic acid (34) as brown solid. Yield: 0.045 g, 15.0%. MS (ES⁻) m/z=450.1 [M−1], (ES⁺) m/z=452.0 [M+1], ¹H NMR (400 MHz, d6-DMSO) δ 13.10 (bs, 1H), 8.03 (dd, 4H, J=54.7, 8.3 Hz), 7.73 (s, 1H), 7.45 (dd, 2H, J=30.1, 3.6 Hz), 7.10 (dd, 4H, J=161.4, 8.6 Hz), 5.18 (s, 2H), 3.72 (s, 3H).

### Example 35. Preparation of (Z)-4-(5-((3-(4-Chlorobenzyl)-4-oxo-2-thioxothiazolidin-5-ylidene)methyl)furan-2-yl)benzoic acid (35)

The title compound was prepared according to Scheme 35.

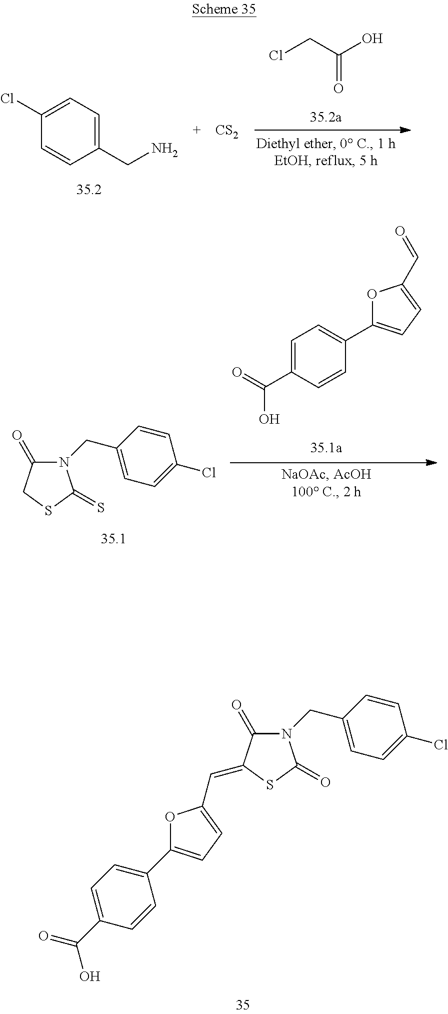

Synthesis of 3-(4-chlorobenzyl)-2-thioxothiazolidin-4-one (35.1). A solution of (4-chlorophenyl)methanamine (35.2, 2.50 g, 17.63 mmol) in diethyl ether (30 mL) under nitrogen was treated with carbon disulfide (1.34 g, 17.63 mmol) at 0° C. The suspension was stirred at 0° C. for 1 h and the solid formed was collected by filtration, washed with diethyl ether and dried under vacuum. The solid obtained was dissolved in ethanol (20 mL) and chloroacetic acid (35.2a, 0.833 g, 8.81 mmol) was added in one portion. The reaction mixture was heated at 100° C. for 2 h. After completion, the mixture was diluted with ice-cold water (20 mL) and extracted with ethyl acetate (50 mL). The organic layer was dried over anhydrous sodium sulfate and concentrated under reduced pressure to get crude. The crude was purified by silica gel (100-200 mesh) column chromatography eluting with 5-10% ethyl acetate in hexanes to afford 3-(4-chlorobenzyl)-2-thioxothiazolidin-4-one (35.1) as a yellow solid. Yield: 1.10 g, 27.5%.

Synthesis of (Z)-4-(5-((3-(4-chlorobenzyl)-4-oxo-2-thioxothiazolidin-5-ylidene)methyl)furan-2-yl)benzoic acid (35). To a solution of 3-(4-chlorobenzyl)-2-thioxothiazolidin-4-one (35.1, 0.150 g, 0.69 mmol) in acetic acid (10 mL), sodium acetate (0.047 g, 0.60 mmol) and 4-(5-formylfuran-2-yl)benzoic acid (35.1a, 0.126 g, 0.69 mmol) were added at room temperature. The reaction mixture was heated at 120° C. for 2 h. After completion, water (20 mL) was added and the precipitate formed was collected by filtration. The product was purified by washings with water, diethyl ether and methanol to afford (Z)-4-(5-((3-(4-chlorobenzyl)-4-oxo-2-thioxothiazolidin-5-ylidene)methyl)furan-2-yl)benzoic acid (35) as brown solid. Yield: 0.127 g, 47.0%. MS (ES⁻) m/z=454.0 [M−1], ¹H NMR (400 MHz, d6-DMSO) δ 13.12 (bs, 1H), 8.03 (dd, 4H, J=53.0, 8.4 Hz), 7.75 (s, 1H), 7.47 (dd, 2H, J=28.7, 3.7 Hz), 7.38 (dd, 4H, J=22.0, 8.5 Hz), 5.24 (s, 2H)

### Example 36. Preparation of (Z)-4-(5-((3-(2-Chlorobenzyl)-4-oxo-2-thioxothiazolidin-5-ylidene)methyl)furan-2-yl)benzoic acid (36)

The title compound was prepared according to Scheme 36.

Synthesis of 3-(2-chlorobenzyl)-2-thioxothiazolidin-4-one (36.1). A solution of (2-chlorophenyl)methanamine (36.2, 1.50 g, 10.5 mmol) in diethyl ether (30 mL) under nitrogen was treated with carbon disulfide (0.80 g, 10.5 mmol) at 0° C. The suspension was stirred at 0° C. for 1 h and the solid formed was collected by filtration, washed with diethyl ether and dried under vacuum. The obtained white solid was dissolved in ethanol (20 mL) and chloroacetic acid (36.2a, 1.0 g, 10.5 mmol) was added in one portion at room temperature. The reaction mixture was then heated at 100° C. for 5 h. After completion of reaction, the mixture was diluted with ice cold water (20 mL) and extracted with ethyl acetate (2×50 mL). The organic layer was dried over anhydrous sodium sulfate and concentrated under reduced pressure. The crude was purified by silica gel (100-200 mesh) column

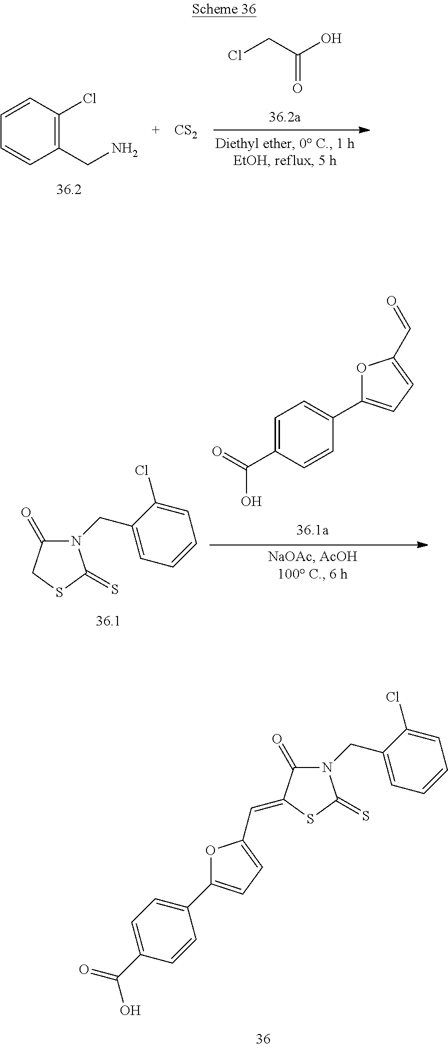

chromatography eluting with 5-10% ethyl acetate in hexanes to afford 3-(2-chlorobenzyl)-2-thioxothiazolidin-4-one (36.1) as a yellow solid. Yield: 0.710 g, 27.0%.

Synthesis of (Z)-4-(5-((3-(2-chlorobenzyl)-4-oxo-2-thioxothiazolidin-5-ylidene)methylguran-2-yl)benzoic acid (36). To a solution of 3-(2-chlorobenzyl)-2-thioxothiazolidin-4-one (36.1, 0.20 g, 0.77 mmol) in acetic acid (10 mL), sodium acetate (0.120 g, 0.77 mmol) and 4-(5-formylfuran-2-yl)benzoic acid (36.1a, 0.168 g, 0.77 mmol) were added at room temperature. The reaction mixture was heated at 120° C. for 6 h. After completion, reaction mixture was cooled, water (20 mL) was added and the precipitate formed was collected by filtration. The product was purified by washings with water, diethyl ether and methanol to afford (Z)-4-(5-((3-(2-chlorobenzyl)-4-oxo-2-thioxothiazolidin-5-ylidene)methyl)furan-2-yl)benzoic acid (36) as brown solid. Yield: 0.151 g, 43.0%. MS (ES⁻) m/z=454.0 [M−1], (ES⁺) m/z=456.1 [M+1], ¹H NMR (400 MHz, d6-DMSO) δ 13.11 (bs, 1H), 8.05 (dd, 4H, J=49.2, 8.3 Hz), 7.79 (s, 1H), 7.51 (d, 1H, J=7.2 Hz), 7.49 (dd, 2H, J=27.4, 3.7 Hz), 7.33 (t, 1H, J=7.3 Hz), 7.28 (t, 1H, J=7.1 Hz), 7.01 (d, 1H, J=7.3 Hz), 5.28 (s, 2H).

### Example 37. Preparation of (Z)-4-(5-((3-(2,4-Dimethylbenzyl)-4-oxo-2-thioxothiazolidin-5-ylidene)methyl)furan-2-yl)benzoic acid (37)

The title compound was prepared according to Scheme 37.

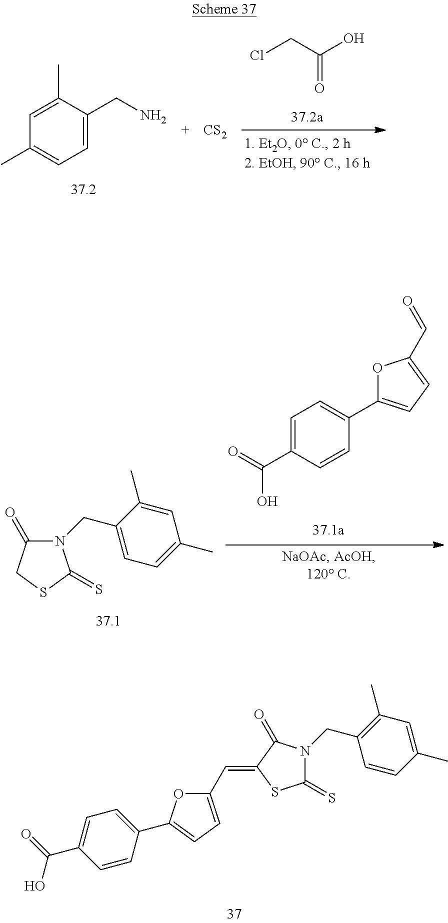

Synthesis of 3-(2,4-dimethylbenzyl)-2-thioxothiazolidin-4-one (37.1). A solution of (2,4-dimethylphenyl)methanamine (37.2, 1.0 g, 7.40 mmol) in diethyl ether (15 mL) under nitrogen was treated with carbon disulfide (0.56 g, 7.40 mmol) at 0° C. The suspension was stirred at 0° C. for 2 h and the precipitate formed was collected by filtration, washed with diethyl ether and dried under vacuum. This solid was dissolved in ethanol (15 mL) and chloroacetic acid (37.2a, 1.04 g, 11.0 mmol) was added in one portion. The reaction mixture was heated at 90° C. for 16 h. The mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(2,4-dimethylbenzyl)-2-thioxothiazolidin-4-one (37.1) as a brown solid. Yield: 0.190 g, 10.60%.

Synthesis of (Z)-4-(5-((3-(2,4-dimethylbenzyl)-4-oxo-2-thioxothiazolidin-5-ylidene)methyl)furan-2-yl)benzoic acid (37). To a solution of 3-(2,4-dimethylbenzyl)-2-thioxothiazolidin-4-one (37.1, 0.170 g, 0.67 mmol) in acetic acid (4 mL) was added sodium acetate (0.056 g, 0.67 mmol) and 4-(5-formylfuran-2-yl) benzoic acid (37.1a, 0.146 g, 0.67 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5-((3-(3,4-dimethylbenzyl)-4-oxo-2-thioxothiazolidin-5-ylidene)methyl)furan-2-yl)benzoic acid (37). Yield: 0.110 g, 36.18%. MS (ES⁻) m/z=448.5 [M-1], (ES⁺) m/z=450.3 [M+1], ¹H NMR (400 MHz, d6-DMSO) δ 13.13 (bs, 1H), 8.04 (dd, 4H, J=49.8, 7.3 Hz), 7.76 (s, 1H), 7.51 (bs, 1H), 7.44 (bs, 1H), 7.03 (s, 1H), 6.81 (dd, 2H, J=80.1, 6.6 Hz) 5.16 (s, 2H), 2.35 (s, 3H), 2.22 (s, 3H)

### Example 38. Preparation of (Z)-4-(5-((3-(3,4-Dimethylbenzyl)-4-oxo-2-thioxothiazolidin-5-ylidene)methyl)furan-2-yl)benzoic acid (38)

The title compound was prepared according to Scheme 38.

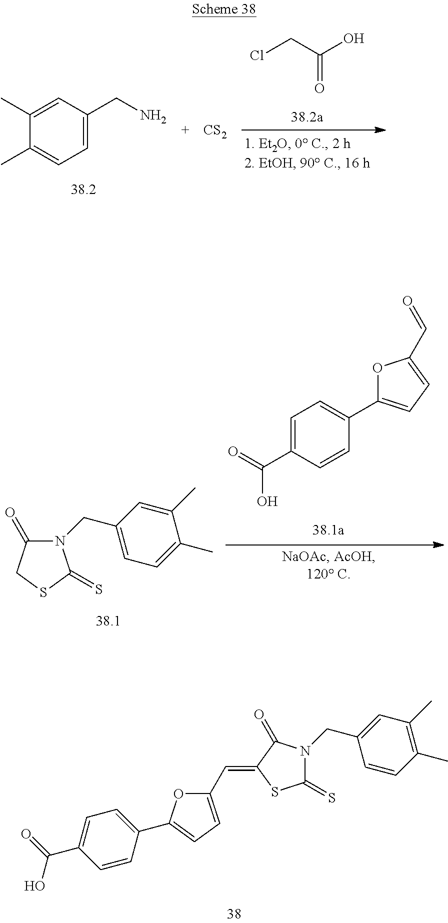

Synthesis of 3-(3,4-dimethylbenzyl)-2-thioxothiazolidin-4-one (38.1). A solution of (3,4-dimethylphenyl)methanamine (38.2, 1.0 g, 7.40 mmol) in diethyl ether (15 mL) under nitrogen was treated with carbon disulfide (0.56 g, 7.40 mmol) at 0° C. The suspension was stirred at 0° C. for 2 h and the precipitate formed was collected by filtration, washed with diethyl ether and dried under vacuum. This solid was dissolved in ethanol (15 mL) and chloroacetic acid (38.2a, 1.04 g, 11.0 mmol) was added in one portion. The reaction mixture was heated at 90° C. for 16 h. The mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(3,4-dimethylbenzyl)-2-thioxothiazolidin-4-one (38.1) as a brown solid. Yield: 0.35 g, 19.40%.

Synthesis of (Z)-4-(5-((3-(3,4-dimethylbenzyl)-4-oxo-2-thioxothiazolidin-5-ylidene)methyl)furan-2-yl)benzoic acid (38). To a solution of 3-(3,4-dimethylbenzyl)-2-thioxothiazolidin-4-one (38.1, 0.170 g, 0.79 mmol) in acetic acid (4 mL) was added sodium acetate (0.083 g, 0.99 mmol) and 4-(5-formylfuran-2-yl)benzoic acid (38.1a, 0.131 g, 0.60 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5-((3-(3,4-dimethylbenzyl)-4-oxo-2-thioxothiazolidin-5-ylidene)methyl)furan-2-yl)benzoic acid (38). Yield: 0.228 g, 75%. MS (ES⁻) m/z=448.4 [M−1], ¹H NMR (400 MHz, d6-DMSO) δ 13.12 (bs, 1H), 8.03 (dd, 4H, J=52.8, 7.6 Hz), 7.74 (s, 1H), 7.50 (bs, 1H), 7.42 (bs, 1H), 7.05 (m, 3H), 5.17 (s, 2H), 2.17 (s, 6H)

### Example 39. Preparation of (Z)-4-(5-((3-(4-Isopropylbenzyl)-4-oxo-2-thioxothiazolidin-5-ylidene)methyl)furan-2-yl)benzoic acid (39)

The title compound was prepared according to Scheme 39.

Synthesis of 3-(4-isopropylbenzyl)-2-thioxothiazolidin-4-one (39.1). A solution of (4-isopropylbenzyl)methanamine (39.2, 1.0 g, 6.80 mmol) in diethyl ether (15 mL) under nitrogen was treated with carbon disulfide (0.56 g, 6.80 mmol) at 0° C. The suspension was stirred at 0° C. for 2 h and the precipitate formed was collected by filtration, washed with diethyl ether and dried under vacuum. This solid was dissolved in ethanol (15 mL) and chloroacetic acid (39.2a, 0.958 g, 10.2 mmol) was added in one portion. The reaction mixture was heated at 90° C. for 16 h. The mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(4-isopropylbenzyl)-2-thioxothiazolidin-4-one (39.1) as a brown solid. Yield: 0.164 g, 10.18%.

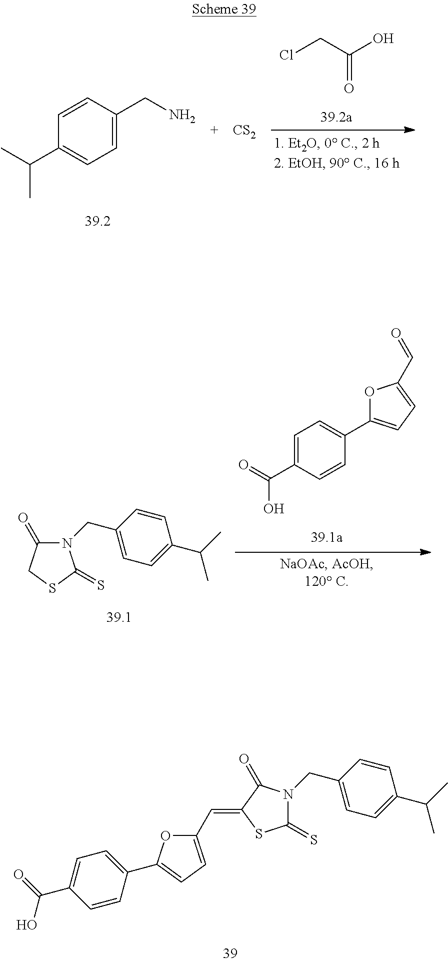

Synthesis of (Z)-4-(5-((3-(4-isopropylbenzyl)-4-oxo-2-thioxothiazolidin-5-ylidene)methyl)furan-2-yl)benzoic acid (39). To a solution of 3-(4-ispropylbenzyl)-2-thioxothiazolidin-4-one (39.1, 0.160 g, 6.0 mmol) in acetic acid (4 mL) was added sodium acetate (0.074 g, 0.90 mmol) and 4-(5-formylfuran-2-yl)benzoic acid (39.1a, 0.117 g, 0.54 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5-((3-(4-isopropylbenzyl)-4-oxo-2-thioxothiazolidin-5-ylidene)methyl)furan-2-yl)benzoic acid (39). Yield: 0.084 g, 30.14%. MS (ES⁻) m/z=462.1 [M−1], (ES⁺) m/z=464.1 [M+1], ¹H NMR (400 MHz, d6-DMSO) δ 13.11 (bs, 1H), 8.04 (dd, 4H, J=32.0, 8.0 Hz), 7.72 (s, 1H), 7.45 (dd, 2H, J=31.1, 3.4 Hz), 7.21 (dd, 4H, J=18.7, 7.4 Hz) 5.19 (s, 2H), 2.83 (septet, 1H, J=6.6 Hz), 1.14 (d, 6H, J=6.6 Hz)

### Example 40. Preparation of (Z)-4-(5-((3-(Cyclohexylmethyl)-4-oxo-2-thioxothiazolidin-5-ylidene)methyl)furan-2-yl)benzoic acid (40)

The title compound was prepared according to Scheme 40.

Synthesis of 3-(cyclohexylmethyl)-2-thioxothiazolidin-4-one (40.1). A solution of cyclohexylmethanamine (40.2, 1.0 g, 8.83 mmol) in diethyl ether (15 mL) under nitrogen was treated with carbon disulfide (0.67 g, 8.83 mmol) at 0° C. The suspension was stirred at

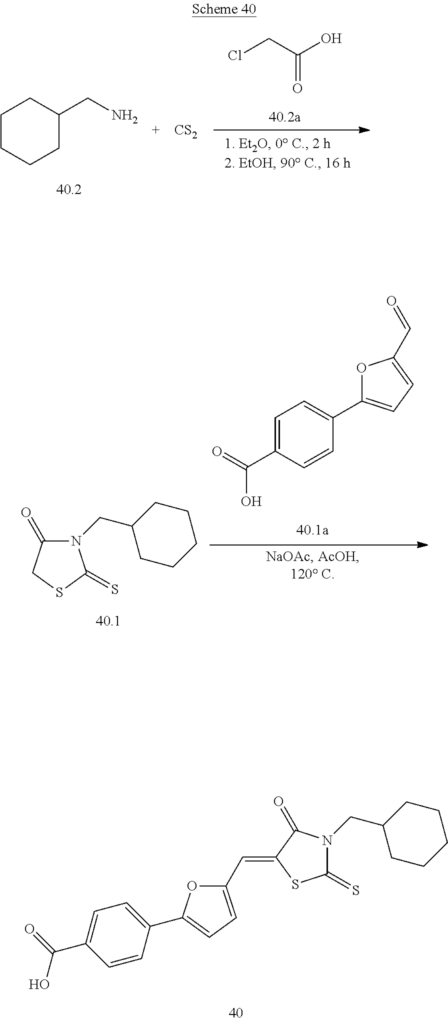

0° C. for 2 h and the precipitate formed was collected by filtration, washed with diethyl ether and dried under vacuum. This solid was dissolved in ethanol (15 mL) and chloroacetic acid (40.2a, 1.25 g, 13.2 mmol) was added in one portion. The reaction mixture was heated at 90° C. for 16 h. After completion, the mixture was diluted with ice-cold water (20 mL) and extracted with ethyl acetate (50 mL). The organic layer was dried over anhydrous sodium sulfate and concentrated under reduced pressure to get the crude product. The crude product was purified by silica gel (100-200 mesh) column chromatography eluting with 20% ethyl acetate in hexanes to afford 3-(cyclohexylmethyl)-2-thioxothiazolidin-4-one (40.1) as a brown solid. Yield: 0.240 g, 11.88%.

Synthesis of (Z)-4-(5-((3-(cyclohexylmethyl)-4-oxo-2-thioxothiazolidin-5-ylidene)methyl)furan-2-yl)benzoic acid (40.1). To a solution of 3-(cyclohexylmethyl)-2-thioxothiazolidin-4-one (40.1, 0.20 g, 0.87 mmol) in acetic acid (4 mL) was added sodium acetate (0.107 g, 1.3 mmol) and 4-(5-formylfuran-2-yl) benzoic acid (40.1a, 0.188 g, 0.87 mmol) at room temperature. The reaction mixture was heated at 120° C. for 16 h. After completion, water (15 mL) was added and a precipitate formed which was collected by filtration. The product was purified by washing with water, diethyl ether and n-pentane to afford (Z)-4-(5-((3-(cyclohexylmethyl)-4-oxo-2-thioxothiazolidin-5-ylidene)methyl)furan-2-yl)benzoic acid (40). Yield: 0.068 g, 18%. MS (ES⁻) m/z=426.1 [M−1], ¹H NMR (400 MHz, d6-DMSO) δ 13.05 (bs, 1H), 8.03 (dd, 4H, J=54.8, 8.2 Hz), 7.07 (s, 1H), 7.45 (dd, 2H, J=34.5, 3.4 Hz), 3.91 (d, 2H, J=7.4 Hz), 1.92 (m, 1H), 1.67 (m, 2H), 1.59 (m, 2H), 1.59 (m, 4H), 1.01 (m, 2H)

### Example 41. Preparation of (Z)-4-(5-((3-(2-hydroxybenzyl)-4-oxo-2-thioxothiazolidin-5-ylidene)methyl)furan-2-yl)benzoic acid (41)

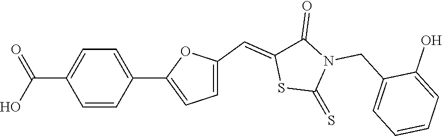 [SMILES: O=C(O)c1ccc(-c2ccc(/C=C3\SC(=S)N(Cc4ccccc4O)C3=O)o2)cc1]

The title compound was prepared as described above, and assayed as described in Example 43.

### Example 42. Preparation of (Z)-5-((5-(4-(1H-tetrazol-5-yl)phenyl)furan-2-yl)methylene)-3-benzyl-2-thioxothiazolidin-4-one (42)

The title compound was prepared according to Scheme 41.

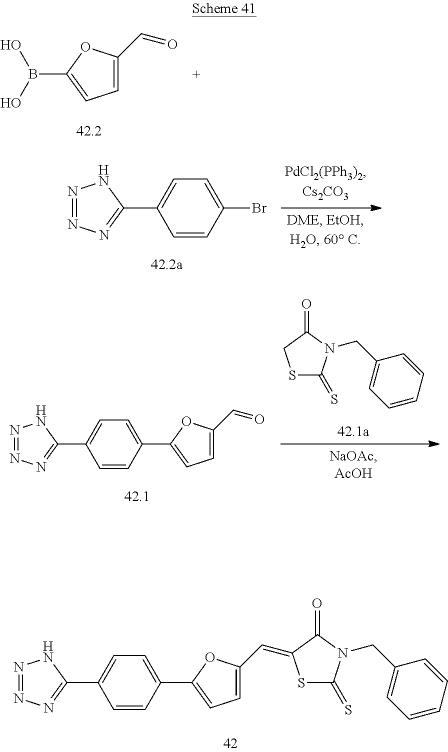 [SMILES: O=Cc1ccc(B(O)O)o1 ; Brc1ccc(-c2nnn[nH]2)cc1 ; O=C1CSC(=S)N1Cc1ccccc1 ; O=Cc1ccc(-c2ccc(-c3nnn[nH]3)cc2)o1 ; O=C1/C(=C/c2ccc(-c3ccc(-c4nnn[nH]4)cc3)o2)SC(=S)N1Cc1ccccc1]

Synthesis of 5-(4-(1H-tetrazol-5-yl)phenyl)furan-2-carbaldehyde (42.1). A solution of 5-(4-bromophenyl)-1H-tetrazole (42.2a, 1.0 g, 4.46 mmol) and (5-formylfuran-2-yl)boronic acid (42.2, 1.89 g 13.5 mmol) in 1,2-dimethoxyethane (10 mL), ethanol (10 mL), and water (2 mL) was prepared at room temperature. The reaction mixture was purged with argon for 15 minutes and PdCl₂(PPh₃)₃ (0.63 g, 0.89 mmol) and cesium carbonate (2.16 g, 11.25 mmol) were added. The reaction mixture was again purged with argon for 10 minutes and reaction was heated at 60° C. for 16 h. After completion, the solvents were removed under reduced pressure and the mixture was diluted with water (40 mL) and filtered through celite. The filtrate was acidified to pH ˜3 with dropwise addition of 50% HCl in water. A precipitate formed which was collected by filtration to give the crude product which was purified by washing with diethyl ether to afford 5-(4-(1H-tetrazol-5-yl)phenyl)furan-2-carbaldehyde (42.1). Yield; 0.86 g, 80.37%.

Synthesis of (Z)-5-((5-(4-(1H-tetrazol-5-yl) phenyl)furan-2-yl)methylene)-3-benzyl-2-thioxothiazolidin-4-one (42). To a solution of 5-(4-(1H-tetrazol-5-yl) phenyl) furan-2-carbaldehyde (42.1, 0.300 g, 1.25 mmol) in acetic acid (10 mL) was added sodium acetate (0.131 g, 1.6 mmol) and 3-benzyl-2-thioxothiazolidin-4-one (42.1a, 0.156 g, 0.72 mmol) at room temperature. The reaction mixture was heated at 120° C. for 48 h. After completion, water (15 mL) was added and a precipitate formed which was collected by filtration. The product was purified by washing with water, diethyl ether and n-pentane to afford (Z)-5-((5-(4-(1H-tetrazol-5-yl) phenyl) furan-2-yl) methylene)-3-benzyl-2-thioxothiazolidin-4-one (42). Yield: 0.284 g, 51.1%. MS (ES⁻) m/z=444.0 [M−1], (ES⁺) m/z=464.1 [M+1], 1H NMR (400 MHz, d6-DMSO) δ 8.14 (dd, 4H, J=55.0, 8.4 Hz), 7.76 (s, 1H), 7.47 (dd, 2H, J=26.4, 3.6 Hz), 7.33 (m, 5H), 5.26 (s, 2H)

### Example 43. Assessment of Integrin Agonist Activity by Cell-Based Assay

Cell Lines. K562 cells (ATCC) stably transfected with wild-type integrin CD11b/CD18 (K562 CD11b/CD18) have been described previously (Park, etal. J Biomol Screen, 2007. 12(3): 406; Gupta, et al., Blood, 2007. 109(8): 3513; incorporated herein by reference in their entirety). Mutant CD11bE320A has been described previously (Alonso, et al. Curr Biol, 2002. 12(10): R340; incorporated herein by reference in its entirety). K562 cells stably transfected with mutant integrin CD11bE320A/CD18 (K562 E320A) were generated according to literature protocols (Park, supra; Gupta, supra). All cell lines were maintained in Iscove's Modified Dulbecco's Medium (IMDM) supplemented with 10% heat-inactivated fetal bovine serum, 50 IU/ml penicillin and streptomycin and 0.5 mg/ml G418.

K562 Cell Adhesion Assay. Cell adhesion assays with immobilized ligands were performed as previously described (Park, supra). Assays with all different K562 cell lines (K562, K562 CD11b/CD18 and K562 E320A) were performed in an identical fashion. Briefly, 384-well Highbind microtiter plates were coated with a 30 μL solution of ligand in Phosphate Buffered Saline, pH 7.4 containing 1 mM each of Ca²⁺ and Mg²⁺ ions (PBS⁺⁺) overnight at 4° C. Ligand Fg was coated at a concentration of 5-15 μg/mL and iC3b at 1-5 μg/mL. Heterodimer specific mAb IB4 was coated at 1-20 μg/mL. Subsequently, the non-specific sites in the wells were blocked by incubation with 1% non-fat milk in Tris Buffered Saline (TBS), pH 7.4, at room temperature for 1 h. Next, the wells were washed three times with TBS. K562 cells were suspended in the assay buffer (TBS containing 1 mM each of Ca²⁺ and Mg²⁺ ions (TBS⁺⁺)) and were transferred to the ligand-coated wells (30,000 cells/well). Stock solutions of the integrin agonists were prepared by dissolving the compounds in DMSO at a concentration of 2-10 mM. Final concentration of DMSO in the assay was approximately 1-2%. K562 cells were incubated in the presence of increasing concentration of integrin agonist for 30 min at 37° C. To dislodge non-adherent cells, the assay plates were gently inverted and kept in the inverted position for 30 min at room temperature. Cells remaining adherent were fixed using formaldehyde and were quantitated using imaging microscopy, as previously described (Park, supra).

The integrin agonists of the invention were found to increase cell adhesion properties. Data from this assay are summarized below in Table 1. Importantly, small structural changes led to unexpected differences in integrin-mediated adhesion. For example, oxazolidine compound 1 was greater than 7-fold more active than imidazolidine compound 4 and greater than 40-fold more active than imidazolidine compound 5 in promoting adhesion. This increase in activity was observed even though the compounds differ only at the position adjacent to the central alkene moiety.

Also advantageously, 4-oxo-2-thioxothiazolidine compounds having a picolinic acid moiety, such as compound 12 and compound 15, exhibited some of the highest activity levels. Compound 12 and compound 15 were more active than most of the other thiazolidine, oxazolidine, and imidazolidine compounds in the study.

TABLE 1
EC50 values for integrin agonists in cell-based adhesion assay.

| Compound # | EC₅₀ |
|---|---|
| 1 | +++ |
| 2 | + |
| 3 | + |
| 4 | ++ |
| 5 | + |
| 6 | + |
| 7 | + |
| 8 | ++++ |
| 9 | + |
| 10 | +++ |
| 11 | + |
| 12 | ++++ |
| 13 | +++ |
| 14 | +++ |
| 15 | ++++ |
| 16 | ++++ |
| 17 | ++ |
| 18 | ++ |
| 19 | ++ |
| 20 | ++ |
| 21 | +++ |
| 22 | ++ |
| 23 | + |
| 24 | + |
| 25 | ++++ |
| 26 | +++ |
| 27 | +++ |
| 28 | +++ |
| 29 | +++ |
| 30 | ++++ |
| 31 | +++ |
| 32 | ++++ |
| 33 | ++++ |
| 34 | ++++ |
| 35 | ++++ |
| 36 | +++ |
| 37 | ++ |
| 38 | ++++ |
| 39 | +++ |
| 40 | ++ |
| 41 | ++ |
| 42 | +++ |

+ = 10 μM < EC₅₀
++ = 1 μM < EC₅₀ ≤ 10 μM
+++ = 250 nM < EC₅₀ ≤ 1 μM
++++ = 100 nM < EC₅₀ ≤ 250 nM

### Example 44. Characterization of Pharmacokinetic Properties for Integrin Agonists in Mice

Integrin agonists as described above were administered to Balb/C mice orally (PO; gavage; 10 mg/kg) and/or intravenously (IV; bolus through tail vein; 1 mg/kg) as a cassette dose with 2 to 3 compounds dosed at a time. A total of twelve mice between age 5-6 weeks with a body weight range of 24-28 g were used. The feeding regimen included 4 h fasting and the feed was provided 2 h after the dosage inoculation and water was provided ad libitum. The blood collection schedule for PO was at 0.25, 0.5, 1, 2, 4, 8,10 and 24 h, and for IV it was at 0.12, 0.25, 0.5, 1, 2, 4, 8 and 24 h post-dose administration under isoflurane anesthesia. For PO dosage, a suspension formulation in 0.2% Tween-80 and 99.8% methyl cellulose (0.5% in water) was prepared in milli-Q water; for IV dosage, a solution formulation in 5% DMSO, 5% Solutol:absolute alcohol (1:1, v/v), and 90% normal saline was prepared.

Dose preparation: For PO dosage, each integrin agonist was wetted with Tween-80 and triturated in a mortar and pestle, and then 0.5% of methyl cellulose was slowly added to make up the final volume for administration. For IV dosage, each integrin agonist was dissolved in DMSO and Solutol:absolute ethanol (1:1, v/v), vortexed, and combined with normal saline.

Methodology for preparation of CC/QC samples: 5 μL of working stock solution was spiked in to 45 μL of blank plasma to obtain the desired concentration in pre-labeled vials. To it was added 250 μL of 100% acetonitrile containing internal standard (tolbutamide: 100 ng/mL) and the sample was vortexed for 2 min followed by centrifugation for 5 min at 20,817×g at 4° C. Supernatant was separated and 5 μL of same was injected on LC-MS/MS.

Data Analysis: Na₂EDTA plasma samples were analyzed using a fit-for-purpose LC-MS/MS method.

The observed pharmacokinetic parameters for selected integrin agonists are shown in Table 2.

TABLE 2
Pharmacokinetic analysis of integrin agonists

| | Compound no. | | | |
|---|---|---|---|---|
| Parameter | 1 | 8 | 12 | 42 |
| Rat Cl (ml/min/kg) | 29.0 | 17.7 | | 19.1 |
| Rat F % (AUC₀₋ₜ | 18.6% | 8.82% | (176) | 4.1% |
| (ng · h/mL)) | (1065) | | | (357) |
| Vdss (L/kg) | 1.5 | 1.37 | | 1.15 |
| t_{1/2} (hr) | 1.97 | 3.09 | 1.39 | 0.77 |
| Tₘₐₓ (hr) | 2.00 | 2.0 | 0.5 | 0.50 |
| Cₘₐₓ (ng/mL) | | | 55.0 | |

### Example 45. Treatment of Colon Cancer with Integrin Agonists in a Mouse Model

Compound 1, compound 8, and compound 12 were dosed at 10 mg/kg BID oral daily for 21 days in a syngeneic colon cancer mouse model using CT-26 cell line. CT26 cells were injected subcutaneously into right flank of female BALB/c mice, aged 7-8 weeks, on Day 1. Tumors were grown for 11 days and dosing was begun and dosed daily for 21 days. Tumor volume/growth (TV=Length×Width 2/2) was measured two times a week. Body weight was measured twice weekly. At the end of study tumor tissue and serum was collected and preserved. Morbidity and Mortality was monitored during the course of study. The results are summarized in Table 3. Each of the compounds reduced tumor volume and/or tumor weight in the colon cancer model.

TABLE 3
Analysis of tumor growth on Day 21 in female Balb/C
dosed with integrin agonists at 10 mg/kg PO BID

| Compound | 1 | 8 | 12 |
|---|---|---|---|
| % Reduction in Tumor Weight | −37.15 | −3.08 | 11.03 |
| % Reduction in Tumor Volume | −38.30 | 5.25 | −4.42 |

### VI. EXEMPLARY EMBODIMENTS

Exemplary embodiments provided in accordance with the presently disclosed subject matter include, but are not limited to, the claims and the following embodiments:
1. A compound according to Formula I:

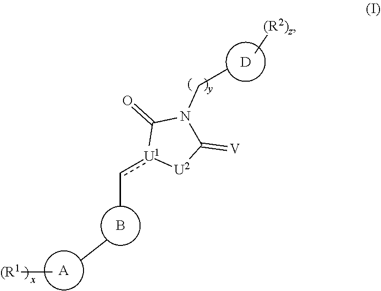

or a pharmaceutically acceptable salt thereof, wherein:
ring A is selected from the group consisting of phenyl and C₆ heteroaryl;
ring B is selected from the group consisting of phenylene and C₅₋₆ heteroarylene, each of which is optionally substituted with one or two C₁₋₄ alkyl;
ring D is selected from the group consisting of cyclohexyl and phenyl;
V is selected from the group consisting of O and S;
U¹ is selected from C, CH, and N;
U² is selected from the group consisting of O, C(R³)(R⁴), and NR^{a};
each R¹ is independently selected from the group consisting of halogen, (CH₂)_{w}COOR^{1a}, and C₅₋₆ heteroaryl, wherein C₅₋₆ heteroaryl is optionally substituted with (CH₂)_{w}COOR^{1a};
each R^{1a} is independently selected from the group consisting of H and C₁₋₆ alkyl;
each subscript w is 0, 1, or 2;
each R² is independently selected from the group consisting of halogen, C₁₋₆ alkyl, C₁₋₆ alkoxy, and R^{2a};
each R^{2a} is independently selected from the group consisting of C₂₋₆ alkyl and C₂₋₆ alkoxy;
each of R³ and R⁴ is independently selected from the group consisting of H and C₁₋₄ alkyl;

R^{a} is selected from the group consisting of H and C₁₋₄ alkyl;
subscript x is 0, 1, 2, 3, 4, or 5;
subscript y is 0, 1, or 2;
subscript z is 0, 1, 2, 3, 4 or 5; and
the dashed line represents a single bond or a double bond;
provided that when V is S, U¹ is C, U² is NH, the dashed line represents a double bond, ring B is furan-2,5-diyl, and (R′)ₓ-(ring A)- is 4-carboxyphenyl,

(R²)_{z}-(ring D)-(CH₂)_{y}— is selected from the group consisting of 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-benzyl, and (R^{2a})_{z}-cyclohexylmethyl, wherein subscript z is 1, 2, or 3;

provided that when V is S, U¹ is C, U² is NH, the dashed line represents a double bond, ring B is furan-2,5-diyl, and (R¹)ₓ-(ring A)- is 4-(COOEt)phenyl,
(R²)_{z}-(ring D)-(CH₂)_{y}— is selected from the group consisting of 4-chlorophenyl, 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxyb enzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-benzyl, and (R^{2a})_{z}-cyclohexylmethyl, wherein subscript z is 1, 2, or 3;
provided that when V is O, U¹ is C, U² is NH, the dashed line represents a double bond, ring B is furan-2,5-diyl, and (R¹)ₓ-(ring A)- is 4-carboxyphenyl,
R²)_{z}-(ring D)-(CH₂)_{y}— is selected from the group consisting of 4-fluorophenyl, 4-methoxyphenyl, 4-methylphenyl, unsubstituted phenethyl, unsubstituted cyclohexymethyl, 3-chlorobenzyl, =2-chlorobenzyl, 3,4-dichlorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{2a})_{z}-phenyl, (R^{2a})_{z}-benzyl, and (R^{2a})_{z}-cyclohexylmethyl, wherein subscript z is 1, 2, or 3; and
provided that when V is O, U¹ is C, U² is S, the dashed line represents a double bond, ring B is furan-2,5-diyl, and (R¹)ₓ-(ring A)- is 4-(COOMe)phenyl or 4-(COOEt)phenyl,
R²)_{z}-(ring D)-(CH₂)_{y}— is selected from the group consisting of 4-chlorophenyl, 4-fluorophenyl, 4-methoxyphenyl, 4-methylphenyl, unsubstituted phenethyl, unsubstituted cyclohexymethyl, 4-chlorobenzyl, 3-chlorobenzyl, 2-chlorobenzyl, 3,4-dichlorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-phenyl, (R^{2a})_{z}-benzyl, and (R^{2a})_{z}-cyclohexylmethyl, wherein subscript z is 1, 2, or 3.
1a. The compound of embodiment 1, wherein subscript x is 1 or 2 and subscript z is 0, 1, 2, or 3.
2. The compound of embodiment 1, having a structure according to Formula II

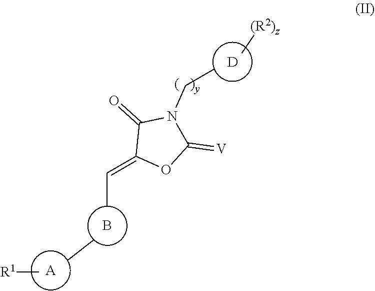

or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.
3. The compound of embodiment 1 or embodiment 2, wherein subscript z is 0, 1, 2, or 3.
4. The compound of any one of embodiments 1-3, wherein ring B is selected from the group consisting of meta-phenylene; thiophene-2,5-diyl; pyridine-2,6-diyl; pyrimidine-2,4-diyl; and furan-2,5-diyl.
5. The compound of any one of embodiments 2-4, wherein ring A is selected from the group consisting of phenyl, pyridin-2-yl, and pyridin-3-yl.
6. The compound of embodiment 5, wherein R^{1a} is selected from the group consisting of COOH and COOMe.
7. The compound of embodiment 2, selected from the group consisting of

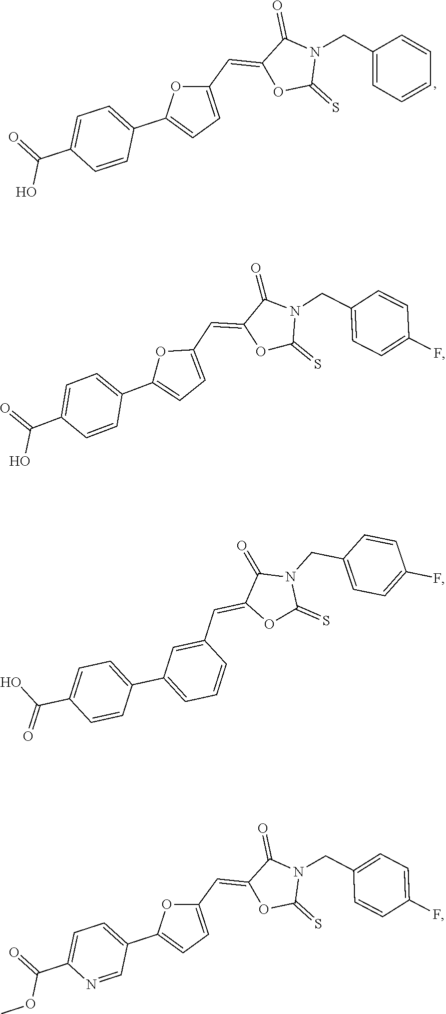 [SMILES: O=C(O)c1ccc(-c2ccc(/C=C3\OC(=S)N(Cc4ccccc4)C3=O)o2)cc1 ; O=C(O)c1ccc(-c2ccc(/C=C3\OC(=S)N(Cc4ccc(F)cc4)C3=O)o2)cc1 ; O=C(O)c1ccc(-c2cccc(/C=C3\OC(=S)N(Cc4ccc(F)cc4)C3=O)c2)cc1 ; COC(=O)c1ccc(-c2ccc(/C=C3\OC(=S)N(Cc4ccc(F)cc4)C3=O)o2)cn1]

and pharmaceutically acceptable salts thereof.
8. The compound of embodiment 1, having a structure according to Formula III

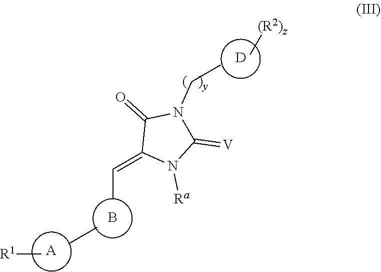

or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.
9. The compound of embodiment 8, wherein subscript z is 0, 1, 2, or 3.
10. The compound of embodiment 8 or embodiment 9, wherein ring B is selected from the group consisting of meta-phenylene; thiophene-2,5-diyl; pyridine-2,6-diyl; pyrimidine-2,4-diyl; and furan-2,5-diyl.
11. The compound of any one of embodiments 8-10, having a structure according to Formula IIIa

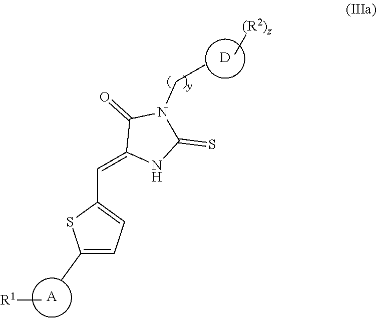

or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.
12. The compound of embodiment 11, wherein subscript z is 0, 1, 2, or 3.
13. The compound of embodiment 8 or embodiment 9, having a structure according to Formula IIIb

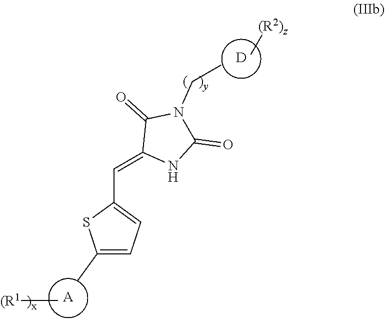

or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.
14. The compound of embodiment 13, wherein subscript xis 1 or 2 and subscript z is 0, 1, 2, or 3.
15. The compound of any one of embodiments 8-14, wherein ring A is selected from the group consisting of phenyl, pyridin-2-yl, and pyridin-3-yl.
16. The compound of embodiment 15, wherein R^{1a} is selected from the group consisting of COOH and COOMe.
17. The compound of embodiment 8, selected from the group consisting of

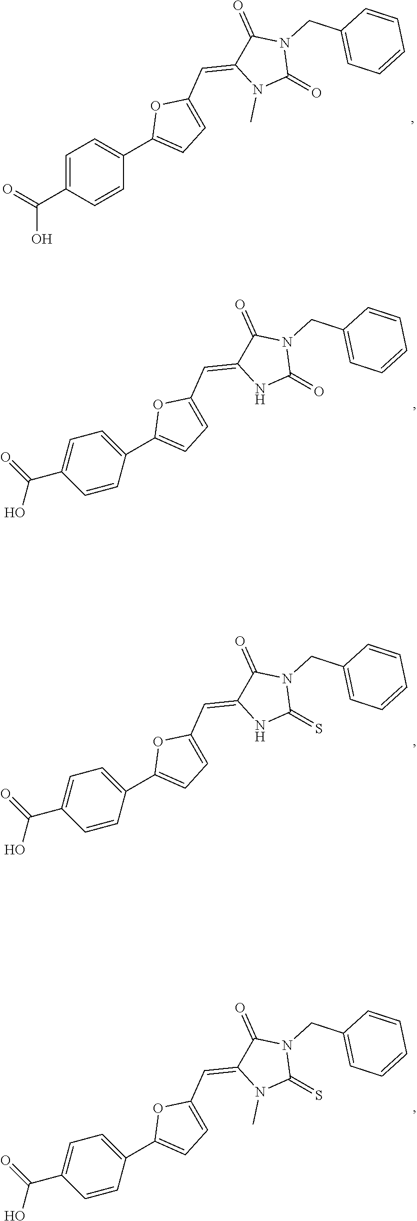

and pharmaceutically acceptable salts thereof.
18. The compound of embodiment 1, having a structure according to Formula IV

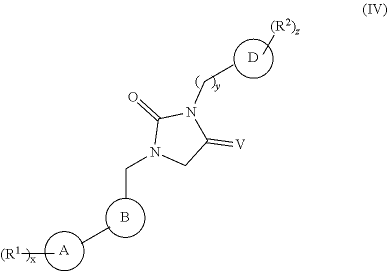

or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.
19. The compound of embodiment 18, wherein subscript xis 1 or 2 and subscript z is 0, 1, 2, or 3.
20. The compound of embodiment 18 or embodiment 19, wherein ring B is selected from the group consisting of meta-phenylene; thiophene-2,5-diyl; pyridine-2,6-diyl; pyrimidine-2,4-diyl; and furan-2,5 -diyl.
21. The compound of any one of embodiments 18-20, wherein ring A is selected from the group consisting of phenyl, pyridin-2-yl, and pyridin-3-yl.
22. The compound of embodiment 21, wherein R^{1a} is selected from the group consisting of COOH and COOMe.
23. The compound of embodiment 18, selected from the group consisting of

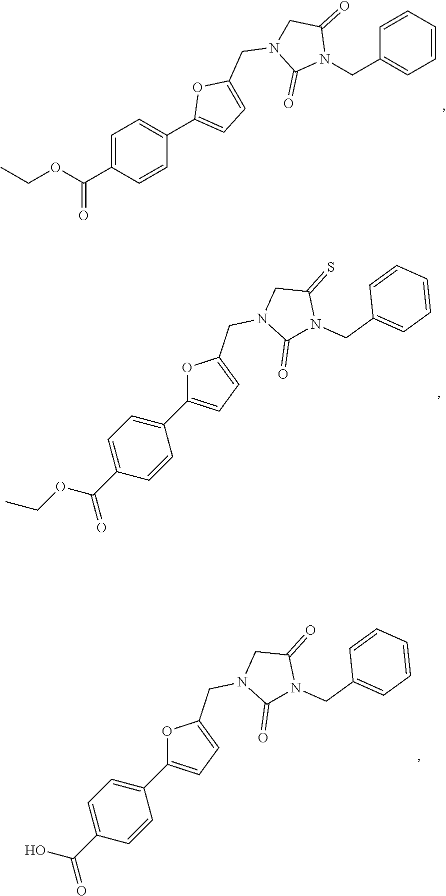 [SMILES: CCOC(=O)c1ccc(-c2ccc(CN3CC(=O)N(Cc4ccccc4)C3=O)o2)cc1 ; "," ; CCOC(=O)c1ccc(-c2ccc(CN3CC(=S)N(Cc4ccccc4)C3=O)o2)cc1 ; "," ; O=C(O)c1ccc(-c2ccc(CN3CC(=O)N(Cc4ccccc4)C3=O)o2)cc1 ; ","]

and pharmaceutically acceptable salts thereof.
24. A compound according to Formula XI:

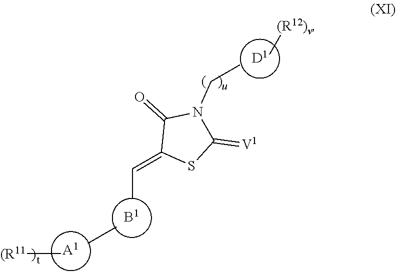

or a pharmaceutically acceptable salt thereof, wherein:
ring A¹ is C₆ heteroaryl;
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen and (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R^{12a};
each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4 or 5.
25. The compound of embodiment 24, wherein subscript t is 1 or 2 and subscript v is 0, 1, 2, or 3.
26. The compound of embodiment 24 or embodiment 25, having a structure according to Formula XIIa

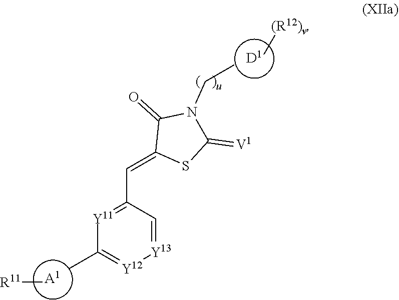

or a pharmaceutically acceptable salt thereof, wherein
R¹¹ is COOR^{11a};
R^{11a} is selected from the group consisting of H and C₁₋₆ alkyl;
each R¹² is independently selected from the group consisting of halogen, C₁₋₆ alkyl, and C₁₋₆ alkoxy; and
Y¹¹, T¹², and Y¹³ are independently selected from the group consisting of CH and N.
27. The compound of embodiment 26, wherein subscript v is 0, 1, 2, or 3.
28. The compound of embodiment 26 or embodiment 27, wherein Y¹¹, Y¹², and Y¹³ are CH.
29. The compound of embodiment 26 or embodiment 27, wherein Y¹¹ is N, and wherein Y¹² and Y¹³ are CH.
30. The compound of embodiment 26 or embodiment 27, wherein Y¹¹ and Y¹² are N, and wherein Y¹³ is CH.
31. The compound of any one of embodiments 26-30, wherein R^{11a} is selected from the group consisting of COOH and COOMe.
32. The compound of embodiment 24 or embodiment 25, having a structure according to Formula XIIb

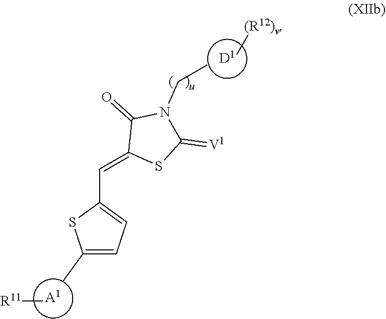

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.
33. The compound of embodiment 32, wherein subscript v is 0, 1, 2, or 3.
34. The compound of embodiment 32 or embodiment 33, wherein ring A¹ is selected from the group consisting of pyridin-2-yl and pyridin-3-yl.
35. The compound of embodiment 24 or embodiment 25, having a structure according to Formula XIIc

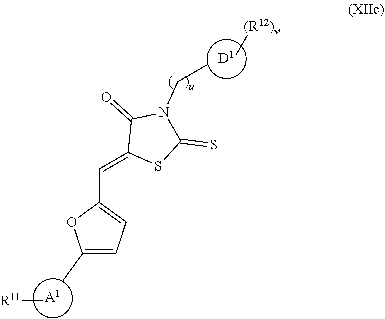

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.
36. The compound of embodiment 35, wherein subscript v is 0, 1, 2, or 3.
37. The compound of embodiment 35 or embodiment 36, wherein ring A¹ is selected from the group consisting of pyridin-2-yl and pyridin-3-yl.
38. The compound of embodiment 35 or embodiment 36, wherein R^{11a} is selected from the group consisting of COOH and COOMe.
39. The compound of embodiment 35, selected from the group consisting of:

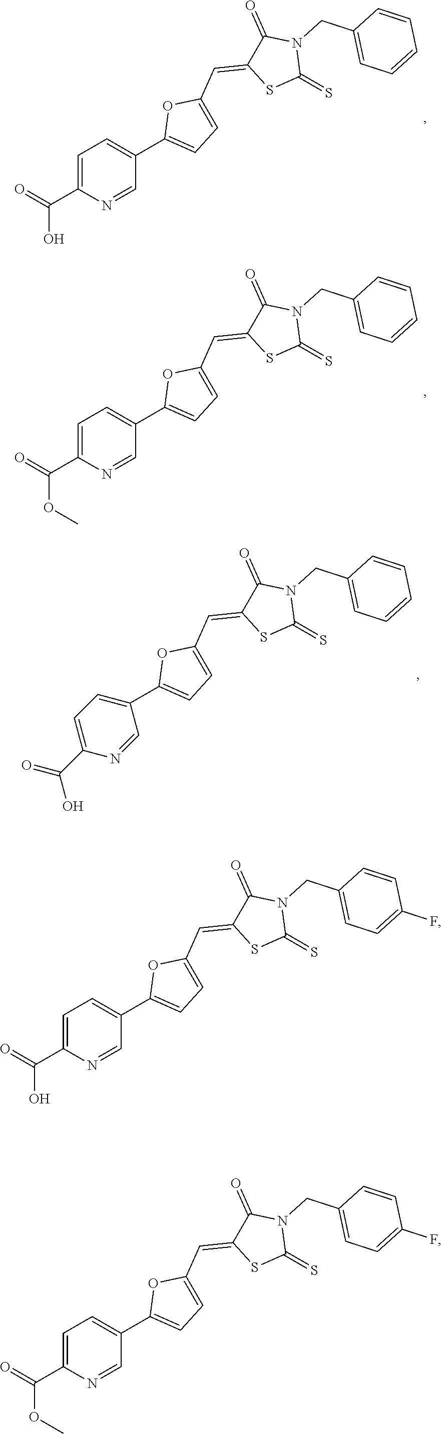

and pharmaceutically acceptable salts thereof.
40. The compound of embodiment 24, having a structure according to Formula XIId

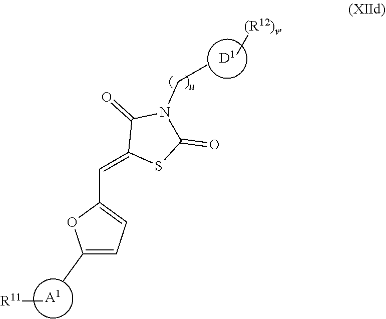

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.
41. The compound of embodiment 40, wherein subscript v is 0, 1, 2, or 3.
42. The compound of embodiment 40 or embodiment 41, wherein ring A¹ is selected from the group consisting of pyridin-2-yl and pyridin-3-yl.
43. A compound according to Formula XIII:

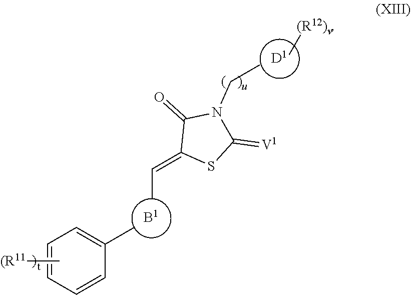

or a pharmaceutically acceptable salt thereof, wherein:
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen, (CH₂)ₛCOOR^{11a}, and C₅₋₆ heteroaryl, wherein C₅₋₆ heteroaryl is optionally substituted with (CH₂)ₛCOOR^{11a}a,
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R^{12a};
each R^{12a} is independently selected from the group consisting of C₂₋₆ alkyl and C₂₋₆ alkoxy;
subscript t is 0,1, 2, 3, 4, or 5;
subscript u is 0, 1, or 2; and
subscript v is 0, 1, 2, 3, 4, or 5;
provided that when V¹ is O, ring B¹ is phen-1,5-diyl, R¹¹ is 4-carboxy, and subscript t is 1,
(R¹²)ᵥ-(ring D¹)-(CH₂)ᵤ— is other than 4-methoxybenzyl;
provided that when V¹ is S, ring B¹ is thiophen-2,5-diyl, is 4-(COOMe), and subscript t is 1,
(R¹²)ᵥ-(ring D¹)-(CH₂)ᵤ— is other than 3,4,5-trimethoxybenzyl;
provided that when V¹ is S, ring B¹ is furan-2,5-diyl, R¹¹ is 4-carboxy, and subscript t is 1,
(R¹²)ᵥ-(ring D¹)-(CH₂)ᵤ— is selected from the group consisting of unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{12a})ᵥ-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOMe), and subscript t is 1,
(R¹²)ᵥ-(ring D¹)-(CH₂)ᵤ— is selected from the group consisting of 4-chlorophenyl, 4-fluorophenyl, 4-methylphenyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{12a})_{z}-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOEt), and subscript t is 1,
(R¹²)ᵥ-(ring D¹)-(CH₂)ᵤ— is selected from the group consisting of unsubstituted cyclohexymethyl, 4-chlorobenzyl, 3-chlorobenzyl, 3,4-dichlorobenzyl, 4-isopropylbenzyl, 3-methoxybenzyl, 2-methoxybenzyl, 2-methylbenzyl, 2,4-dimethylbenzyl, 3,4-dimethylbenzyl, 2,3-dimethylbenzyl, (R^{12a})ᵥ-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOnBu), and subscript t is 1,
(R¹²)ᵥ-(ring D¹)-(CH₂)ᵤ— is selected from the group consisting of unsubstituted phenethyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3 -methylbenzyl, 2-methylbenzyl, 2,4-dimethylbenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, is 4-carboxy, and subscript t is 1,
(R¹²)ᵥ-(ring D¹)-(CH₂)ᵤ— is selected from the group consisting of 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-(COOMe), and subscript t is 1,
(R¹²)ᵥ-(ring D¹)-(CH₂)ᵤ— is selected from the group consisting of 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-(COOEt), and subscript t is 1,
(R¹²)ᵥ-(ring D¹)-(CH₂)ᵤ— is selected from the group consisting of 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, is 4-(COOnBu), and subscript t is 1,
(R¹²)ᵥ-(ring D¹)-(CH₂)ᵤ— is selected from the group consisting of 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3 -dimethylbenzyl, (R^{12a})ᵤ-phenyl, (R^{12a})ᵤ-benzyl, and (R^{12a})ᵤ-cyclohexylmethyl, wherein subscript u is 1, 2, or 3.
43a. The compound of embodiment 43, wherein subscript t is 1 or 2 and subscript v is 0, 1, 2, or 3.
44. The compound of embodiment 43, having a structure according to Formula XIVa

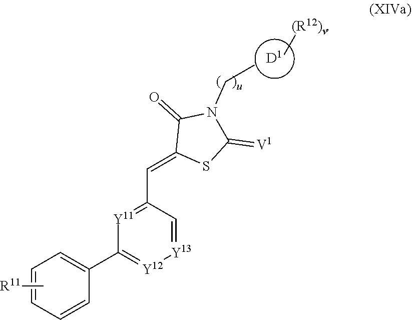

or a pharmaceutically acceptable salt thereof, wherein
R¹¹ is COOR^{11a};
R^{11a} is selected from the group consisting of H and C₁₋₆ alkyl;
each R¹² is independently selected from the group consisting of halogen, C₁₋₆ alkyl, and C₁₋₆ alkoxy; and
Y¹¹, Y¹², and Y¹³ are independently selected from the group consisting of CH and N.
45. The compound of embodiment 43 or embodiment 44, wherein subscript v is 0, 1, 2, or 3.
46. The compound of embodiment 44 or embodiment 45, wherein Y¹¹, Y¹², and Y¹³ are CH.
47. The compound of embodiment 44 or embodiment 45, wherein Y¹¹ is N, and wherein Y¹² and Y¹³ are CH.
48. The compound of embodiment 44 or embodiment 45, wherein Y¹¹ and Y¹² are N, and wherein Y¹³ is CH.
49. The compound of any one of embodiments 44-48, wherein R^{11a} is selected from the group consisting of COOH and COOMe.
50. The compound of embodiment 43, selected from the group consisting of

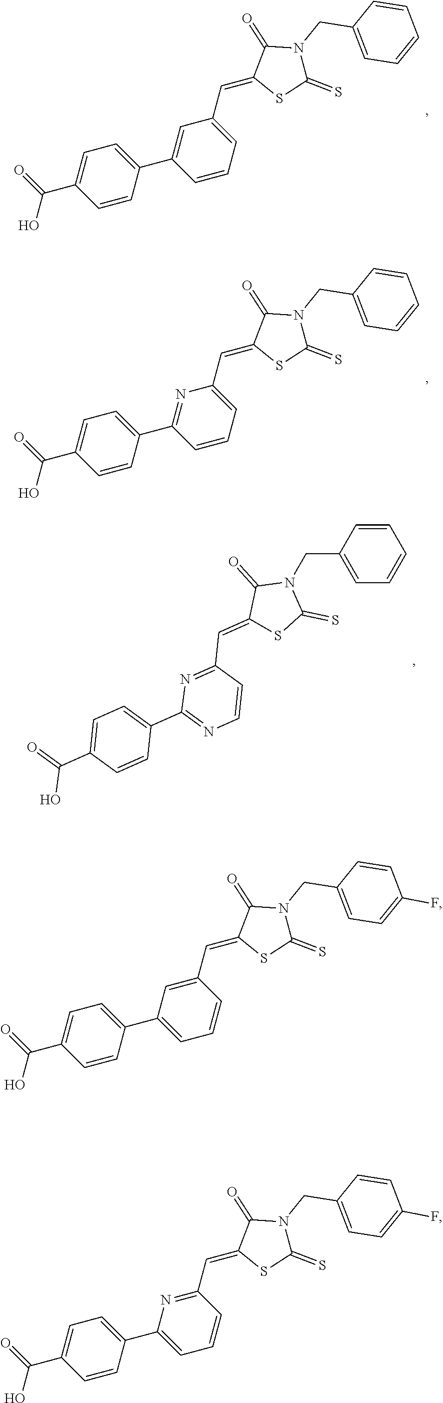

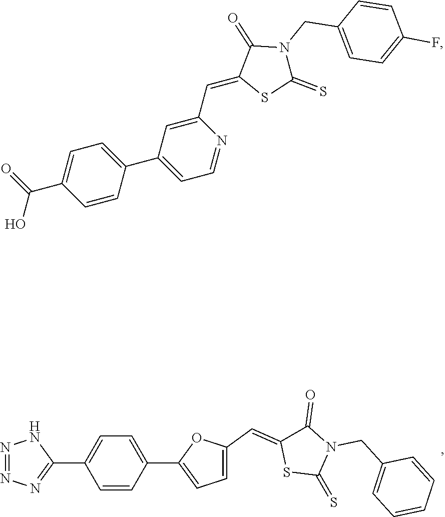 [SMILES: O=C(O)c1ccc(-c2ccnc(/C=C3\SC(=S)N(Cc4ccc(F)cc4)C3=O)c2)cc1 ; O=C1/C(=C/c2ccc(-c3ccc(-c4nnn[nH]4)cc3)o2)SC(=S)N1Cc1ccccc1 ; ","]

and pharmaceutically acceptable salts thereof.
51. The compound of embodiment 43, selected from the group consisting of

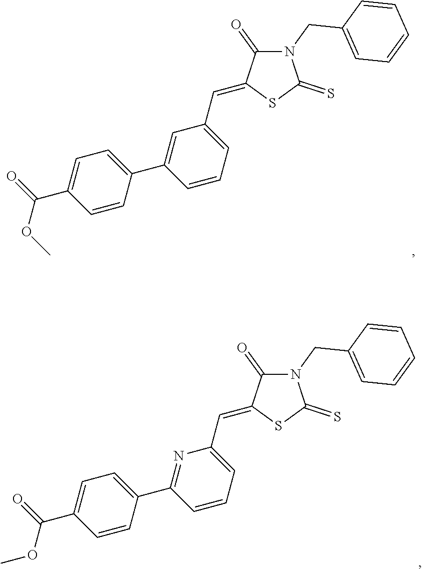 [SMILES: COC(=O)c1ccc(-c2cccc(/C=C3\SC(=S)N(Cc4ccccc4)C3=O)c2)cc1 ; COC(=O)c1ccc(-c2cccc(/C=C3\SC(=S)N(Cc4ccccc4)C3=O)n2)cc1]

and pharmaceutically acceptable salts thereof.
52. The compound of embodiment 43, having a structure according to Formula XIVb

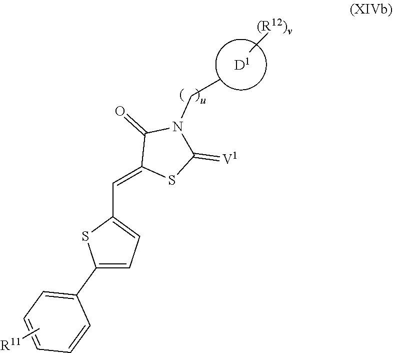

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.
53. The compound of embodiment 52, wherein subscript v is 0, 1, 2, or 3.
54. The compound of embodiment 52 or embodiment 53, which is

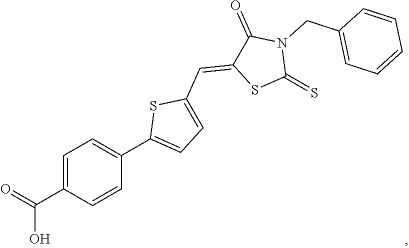 [SMILES: O=C(O)c1ccc(-c2ccc(/C=C3\SC(=S)N(Cc4ccccc4)C3=O)s2)cc1]

or a pharmaceutically acceptable salt thereof.
55. The compound of embodiment 43, having a structure according to Formula XIVc

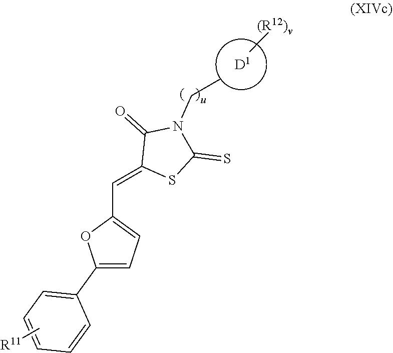

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.
56. The compound of embodiment 55, wherein subscript v is 0, 1, 2, or 3.
57. The compound of embodiment 55 or embodiment 56, wherein R^{11a} is selected from the group consisting of COOH and COOMe.
58. The compound of embodiment 55, selected from the group consisting of:

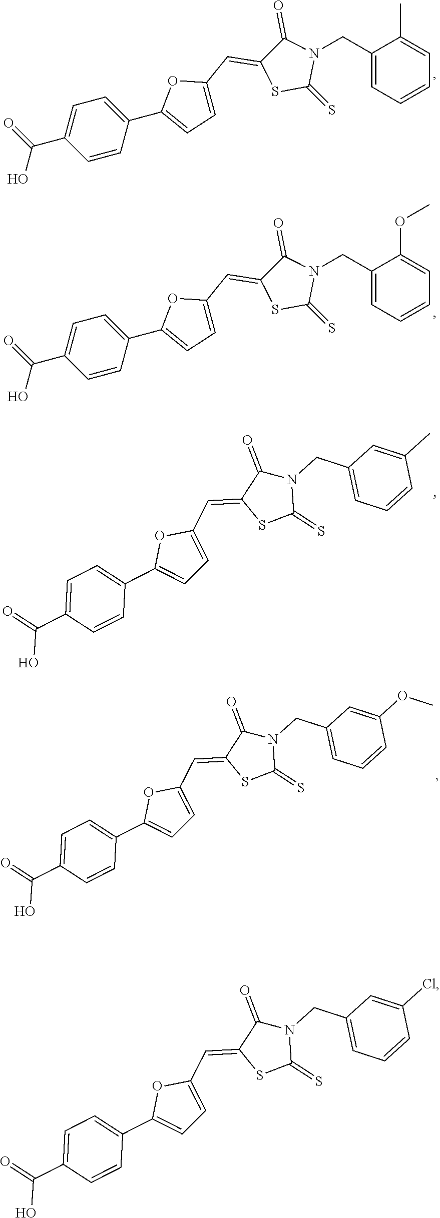 [SMILES: Cc1ccccc1CN1C(=O)/C(=C/c2ccc(-c3ccc(C(=O)O)cc3)o2)SC1=S ; COc1ccccc1CN1C(=O)/C(=C/c2ccc(-c3ccc(C(=O)O)cc3)o2)SC1=S ; Cc1cccc(CN2C(=O)/C(=C/c3ccc(-c4ccc(C(=O)O)cc4)o3)SC2=S)c1 ; COc1cccc(CN2C(=O)/C(=C/c3ccc(-c4ccc(C(=O)O)cc4)o3)SC2=S)c1 ; O=C(O)c1ccc(-c2ccc(/C=C3\SC(=S)N(Cc4cccc(Cl)c4)C3=O)o2)cc1]

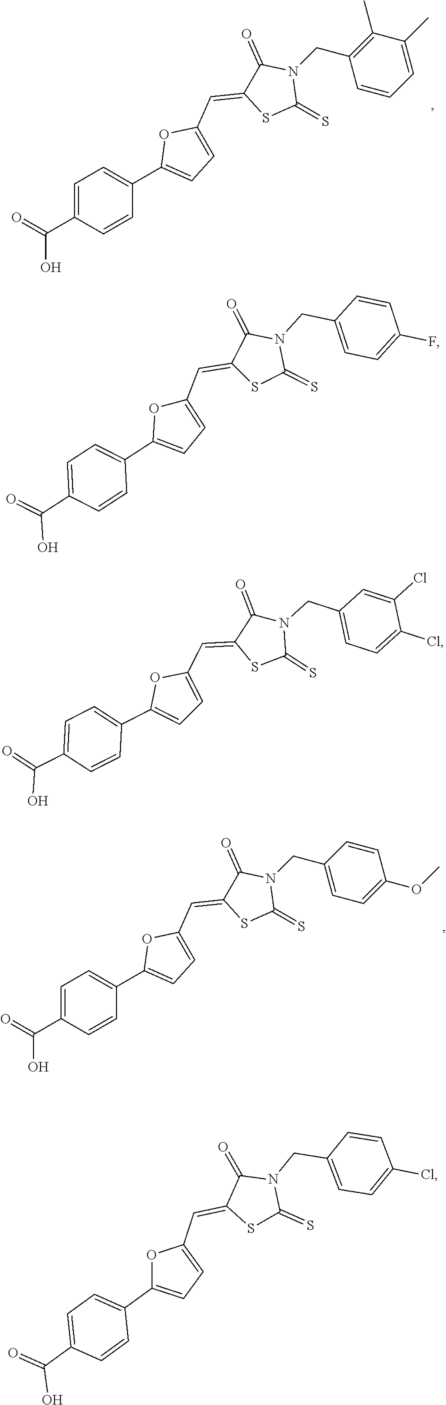 [SMILES: Cc1cccc(CN2C(=O)/C(=C/c3ccc(-c4ccc(C(=O)O)cc4)o3)SC2=S)c1C ; O=C(O)c1ccc(-c2ccc(/C=C3\SC(=S)N(Cc4ccc(F)cc4)C3=O)o2)cc1 ; O=C(O)c1ccc(-c2ccc(/C=C3\SC(=S)N(Cc4ccc(Cl)c(Cl)c4)C3=O)o2)cc1 ; COc1ccc(CN2C(=O)/C(=C/c3ccc(-c4ccc(C(=O)O)cc4)o3)SC2=S)cc1 ; O=C(O)c1ccc(-c2ccc(/C=C3\SC(=S)N(Cc4ccc(Cl)cc4)C3=O)o2)cc1]

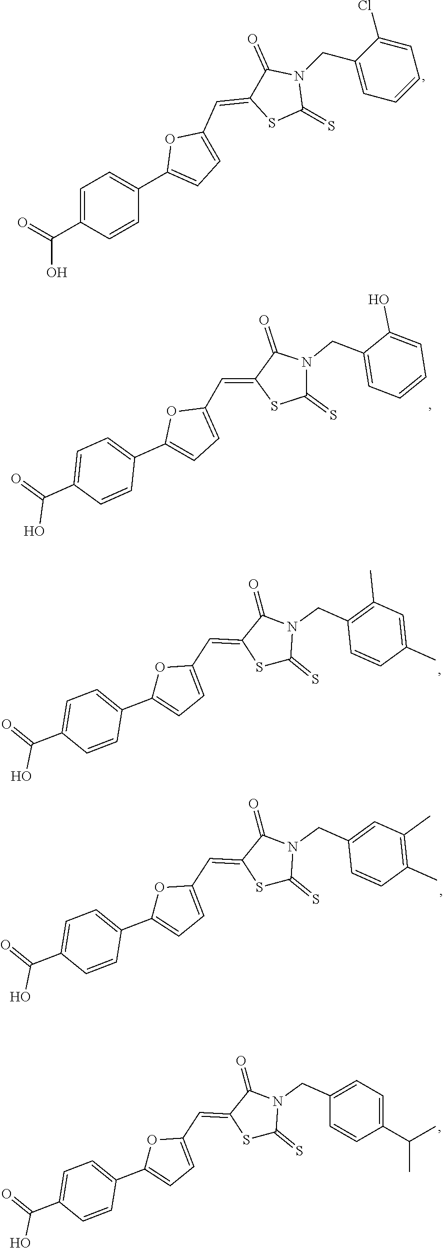 [SMILES: O=C(O)c1ccc(-c2ccc(/C=C3\SC(=S)N(Cc4ccccc4Cl)C3=O)o2)cc1 ; O=C(O)c1ccc(-c2ccc(/C=C3\SC(=S)N(Cc4ccccc4O)C3=O)o2)cc1 ; Cc1ccc(CN2C(=O)/C(=C/c3ccc(-c4ccc(C(=O)O)cc4)o3)SC2=S)c(C)c1 ; Cc1ccc(CN2C(=O)/C(=C/c3ccc(-c4ccc(C(=O)O)cc4)o3)SC2=S)cc1C ; CC(C)c1ccc(CN2C(=O)/C(=C/c3ccc(-c4ccc(C(=O)O)cc4)o3)SC2=S)cc1]

and pharmaceutically acceptable salts thereof.
59. The compound of embodiment 55, having the structure

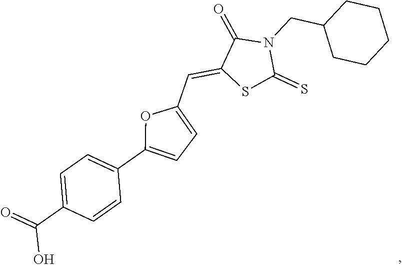 [SMILES: O=C(O)c1ccc(-c2ccc(/C=C3\SC(=S)N(CC4CCCCC4)C3=O)o2)cc1]

and pharmaceutically acceptable salts thereof.
60. The compound of embodiment 43, having a structure according to Formula XIVd

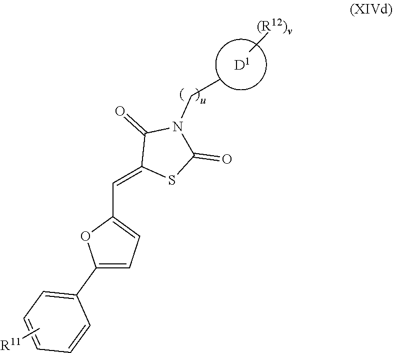

or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.
61. The compound of embodiment 60, wherein subscript v is 0, 1, 2, or 3.
62. A pharmaceutical formulation comprising a pharmaceutically acceptable excipient and a compound according to any one of embodiments 1-61 or a pharmaceutically acceptable salt thereof.
63. A method for treating a β2 integrin-mediated condition comprising administering to a patient in need thereof a compound according to any one of embodiments 1-61 or a pharmaceutically acceptable salt thereof.
64. The method of embodiment 63, wherein the integrin-mediated condition is selected from the group consisting of acute inflammation, chronic inflammation, chronic kidney disease, neointimal thickening associated with vascular injury, atherosclerosis, tissue injury, peritonitis, diabetic nephropathy, an autoimmune disease, a neurological condition, lupus, cancer, pain, glaucoma, graft versus host disease, macular degeneration, and uveitis.
65. The method of embodiment 63, wherein the β2 integrin-mediated condition is cancer.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications, websites, and databases cited herein are hereby incorporated by reference in their entireties for all purposes.

What is claimed is:

1. A compound according to Formula I: 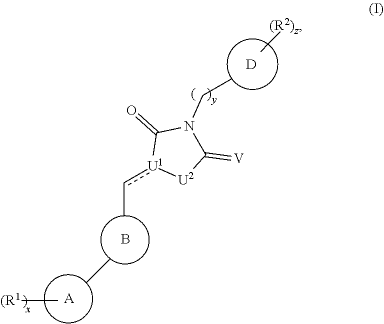
or a pharmaceutically acceptable salt thereof, wherein:
ring A is selected from the group consisting of phenyl and C₆ heteroaryl;
ring B is selected from the group consisting of C₅₋₆ heteroarylene and phenylene, each of which is unsubstituted or optionally substituted with one or two C₁₋₄ alkyl;
ring D is selected from the group consisting of phenyl and cyclohexyl;
V is selected from the group consisting of S and O;
U¹ is selected from C, CH, and N;
U² is selected from the group consisting of O, C(R³)(R⁴), and NR^{a};
each R¹ is independently selected from the group consisting of (CH₂)_{w}COOR^{1a}, halogen, and C₅₋₆ heteroaryl, wherein C₅₋₆ heteroaryl is optionally substituted with (CH₂)_{w}COOR^{1a};
each R^{1a} is independently selected from the group consisting of H and C₁₋₆ alkyl;
each subscript w is 0, 1, or 2;
each R² is independently selected from the group consisting of halogen, C₁₋₆ alkyl, C₁₋₆ alkoxy, and Rea;
each R^{2a} is independently selected from the group consisting of C₂₋₆ alkyl and C₂₋₆ alkoxy;
each of R³ and R⁴ is independently selected from the group consisting of H and C₁₋₄ alkyl;
R^{a} is selected from the group consisting of H and C₁₋₄ alkyl;
subscript x is 1, 0, 2, 3, 4, or 5;
subscript y is 1, 0, or 2;
subscript z is 0, 1, 2, 3, 4, or 5; and
the dashed line represents a double bond or a single bond;
provided that when V is S, U¹ is C, U² is NH, the dashed line represents a double bond, ring B is furan-2,5-diyl, and (R¹)ₓ-(ring A)- is 4-carboxyphenyl,
(R²)_{z}-(ring D)-(CH₂)_{y}— is selected from the group consisting of 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-benzyl, and (R^{2a})_{z}-cyclohexylmethyl, wherein subscript z is 1, 2, or 3;
provided that when V is S, U¹ is C, U² is NH, the dashed line represents a double bond, ring B is furan-2,5-diyl, and (R¹)ₓ-(ring A)- is 4-(COOEt)phenyl,
(R²)_{z}-(ring D)-(CH₂)_{y}— is selected from the group consisting of 4-chlorophenyl, 4-fluorophenyl, 4-methoxyphenyl, 4-methylphenyl, unsubstituted phenethyl, unsubstituted cyclohexymethyl, unsubstituted benzyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-benzyl, and (R^{2a})_{z}-cyclohexylmethyl, wherein subscript z is 1, 2, or 3;
provided that when V is O, U¹ is C, U² is NH, the dashed line represents a double bond, ring B is furan-2,5-diyl, and (R¹)ₓ-(ring A)- is 4-carboxyphenyl,
R²)_{z}-(ring D)-(CH₂)_{y}— is selected from the group consisting of 4-fluorophenyl, 4-methoxyphenyl, 4-methylphenyl, unsubstituted phenethyl, unsubstituted cyclohexymethyl, 3-chlorobenzyl,=2-chlorobenzyl, 3,4-dichlorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{2a})_{z}-phenyl, (R^{2a})_{z}-benzyl, and (R^{2a})_{z}-cyclohexylmethyl, wherein subscript z is 1, 2, or 3; and
provided that when V is O, U¹ is C, U² is S, the dashed line represents a double bond, ring B is furan-2,5-diyl, and (R¹)ₓ-(ring A)- is 4-(COOMe)phenyl or 4-(COOEt)phenyl,
R²)_{z}-(ring D)-(CH₂)_{y}— is selected from the group consisting of 4-chlorophenyl, 4-fluorophenyl, 4-methoxyphenyl, 4-methylphenyl, unsubstituted phenethyl, unsubstituted cyclohexymethyl, 4-chlorobenzyl, 3-chlorobenzyl, 2-chlorobenzyl, 3,4-dichlorobenzyl, 4-isopropylbenzyl, 4-methoxybenzyl, 3 -methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{2a})_{z}-phenyl, (R^{2a})_{z}-benzyl, and (R^{2a})_{z}-cyclohexylmethyl, wherein subscript z is 1, 2, or 3.

2. The compound of claim 1, having a structure according to Formula II 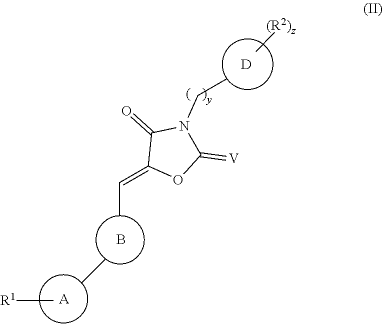
or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.

3. The compound of claim 2, wherein subscript z is 0, 1, 2, or 3.

4. The compound of claim 2, wherein ring B is selected from the group consisting of meta-phenylene; thiophene-2,5-diyl; pyridine-2,6-diyl; pyrimidine-2,4-diyl; and furan-2,5-diyl.

5. The compound of claim 2, wherein ring A is selected from the group consisting of phenyl, pyridin-2-yl, and pyridin-3-yl.

6. The compound of claim 5, wherein R^{1a} is selected from the group consisting of COOH and COOMe.

7. The compound of claim 2, selected from the group consisting of 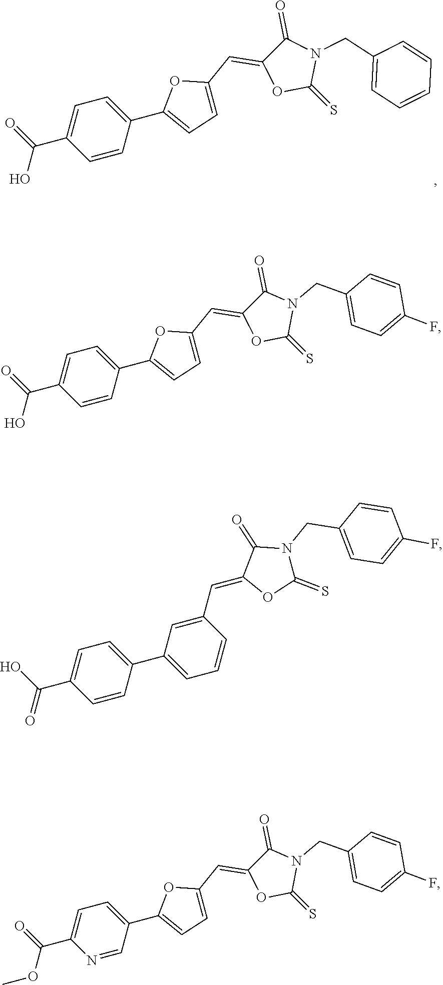 [SMILES: O=C(O)c1ccc(-c2ccc(/C=C3\OC(=S)N(Cc4ccccc4)C3=O)o2)cc1 ; O=C(O)c1ccc(-c2ccc(/C=C3\OC(=S)N(Cc4ccc(F)cc4)C3=O)o2)cc1 ; O=C(O)c1ccc(-c2cccc(/C=C3\OC(=S)N(Cc4ccc(F)cc4)C3=O)c2)cc1 ; COC(=O)c1ccc(-c2ccc(/C=C3\OC(=S)N(Cc4ccc(F)cc4)C3=O)o2)cn1]
and pharmaceutically acceptable salts thereof.

8. The compound of claim 1, having a structure according to Formula III 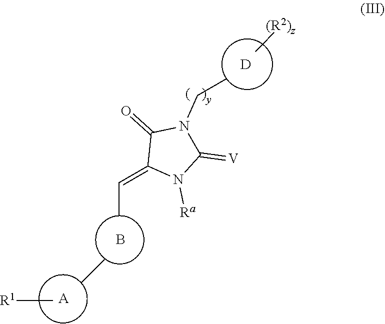
or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.

9. The compound of claim 8, wherein subscript z is 0, 1, 2, or 3.

10. The compound of claim 8, wherein ring B is selected from the group consisting of meta-phenylene; thiophene-2,5-diyl; pyridine-2,6-diyl; pyrimidine-2,4-diyl; and furan-2,5-diyl.

11. The compound of claim 8, having a structure according to Formula IIIa 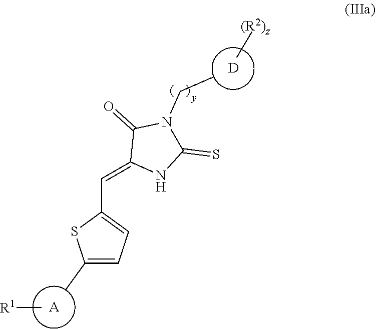
or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.

12. The compound of claim 11, wherein subscript z is 0, 1, 2, or 3.

13. The compound of claim 8, having a structure according to Formula IIIb 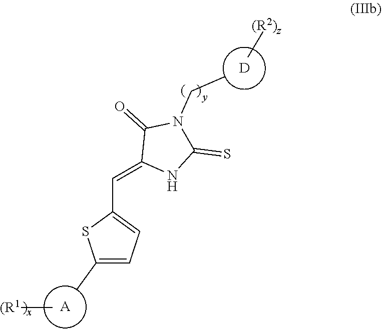
or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.

14. The compound of claim 13, wherein subscript x is 1 or 2 and subscript z is 0, 1, 2, or 3.

15. The compound of claim 8, wherein ring A is selected from the group consisting of phenyl, pyridin-2-yl, and pyridin-3-yl.

16. The compound of claim 15, wherein R^{1a} is selected from the group consisting of COOH and COOMe.

17. The compound of claim 8, selected from the group consisting of 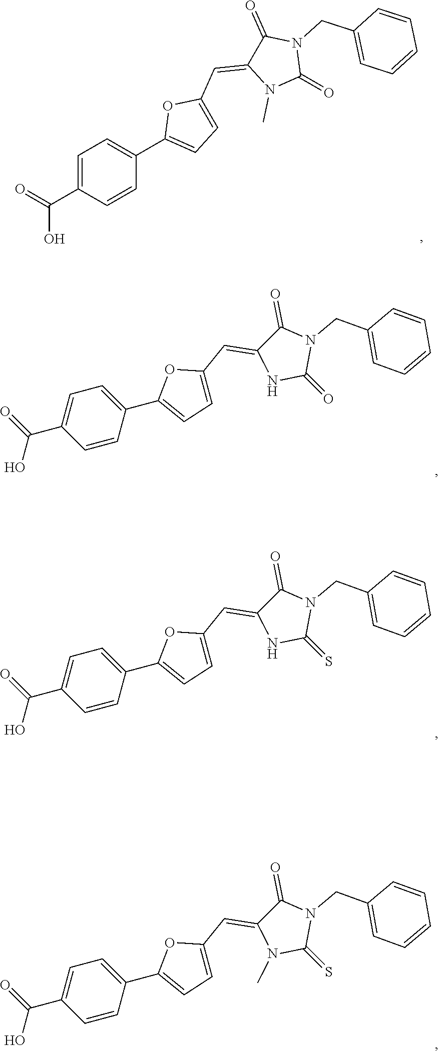 [SMILES: CN1C(=O)N(Cc2ccccc2)C(=O)/C1=C/c1ccc(-c2ccc(C(=O)O)cc2)o1 ; O=C(O)c1ccc(-c2ccc(/C=C3\NC(=O)N(Cc4ccccc4)C3=O)o2)cc1 ; O=C(O)c1ccc(-c2ccc(/C=C3\NC(=S)N(Cc4ccccc4)C3=O)o2)cc1 ; CN1C(=S)N(Cc2ccccc2)C(=O)/C1=C/c1ccc(-c2ccc(C(=O)O)cc2)o1]
and pharmaceutically acceptable salts thereof.

18. The compound of claim 1, having a structure according to Formula IV 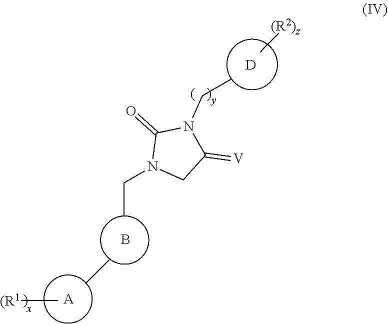
or a pharmaceutically acceptable salt thereof, wherein
R¹ is COOR^{1a}, wherein R^{1a} is selected from the group consisting of H and C₁₋₆ alkyl; and
each R² is independently selected from the group consisting of halogen, C₁₋₆ alkyl, and C₁₋₆ alkoxy.

19. The compound of claim 18, wherein subscript x is 1 or 2 and subscript z is 0, 1, 2, or 3.

20. The compound of claim 18, wherein ring B is selected from the group consisting of meta-phenylene; thiophene-2,5-diyl; pyridine-2,6-diyl; pyrimidine-2,4-diyl; and furan-2,5-diyl.

21. The compound of claim 18, wherein ring A is selected from the group consisting of phenyl, pyridin-2-yl, and pyridin-3-yl.

22. The compound of claim 21, wherein R^{1a} is selected from the group consisting of COOH and COOMe.

23. The compound of claim 18, selected from the group consisting of 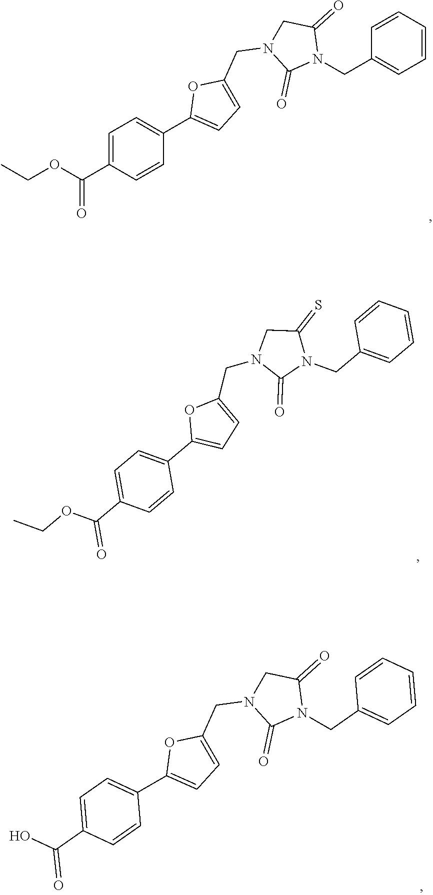 [SMILES: CCOC(=O)c1ccc(-c2ccc(CN3CC(=O)N(Cc4ccccc4)C3=O)o2)cc1 ; CCOC(=O)c1ccc(-c2ccc(CN3CC(=S)N(Cc4ccccc4)C3=O)o2)cc1 ; O=C(O)c1ccc(-c2ccc(CN3CC(=O)N(Cc4ccccc4)C3=O)o2)cc1]
and pharmaceutically acceptable salts thereof.

24. A compound according to Formula XI: 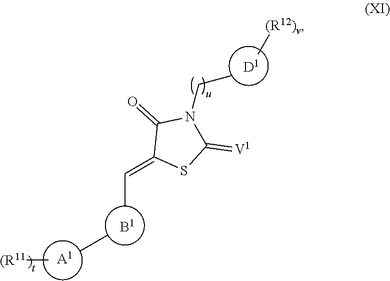
or a pharmaceutically acceptable salt thereof, wherein:
ring A¹ is C₆ heteroaryl;
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R¹¹ is independently selected from the group consisting of halogen and (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R^{12a};
each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4, or 5.

25. The compound of claim 24, wherein subscript t is 1 or 2 and subscript v is 0, 1, 2, or 3.

26. The compound of claim 24, having a structure according to Formula XIIa 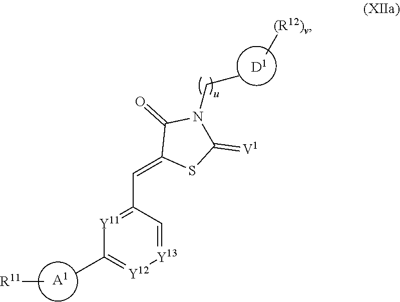
or a pharmaceutically acceptable salt thereof, wherein
R¹¹ is COOR^{11a};
R^{11a} is selected from the group consisting of H and C₁₋₆ alkyl;
each R¹² is independently selected from the group consisting of halogen, C₁₋₆ alkyl, and C₁₋₆ alkoxy; and
Y¹¹, Y¹², and Y¹³ are independently selected from the group consisting of CH and N.

27. The compound of claim 26, wherein subscript v is 0, 1, 2, or 3.

28. The compound of claim 26, wherein Y¹¹, Y¹², and Y¹³ are CH.

29. The compound of claim 26, wherein Y¹¹ is N, and wherein Y¹² and Y¹³ are CH.

30. The compound of claim 26, wherein Y¹¹ and Y¹² are N, and wherein Y¹³ is CH.

31. The compound of claim 26, wherein R^{11a} is selected from the group consisting of COOH and COOMe.

32. The compound of claim 24, having a structure according to Formula XIIb 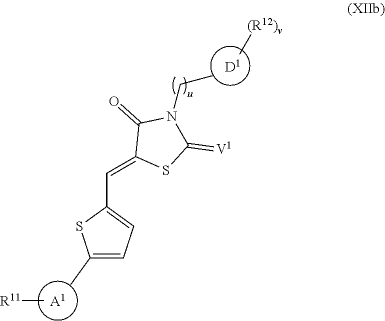
or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

33. The compound of claim 32, wherein subscript v is 0, 1, 2, or 3.

34. The compound of claim 32, wherein ring A¹ is selected from the group consisting of pyridin-2-yl and pyridin-3-yl.

35. The compound of claim 24, having a structure according to Formula XIIc 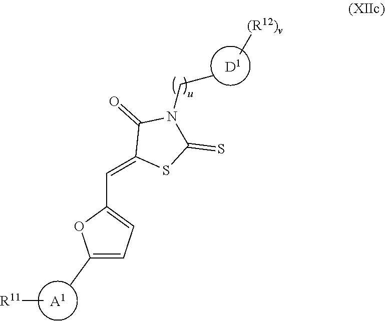
or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

36. The compound of claim 35, wherein subscript v is 0, 1, 2, or 3.

37. The compound of claim 35, wherein ring A¹ is selected from the group consisting of pyridin-2-yl and pyridin-3-yl.

38. The compound of claim 35, wherein R^{11a} is selected from the group consisting of COOH and COOMe.

39. The compound of claim 35, selected from the group consisting of: 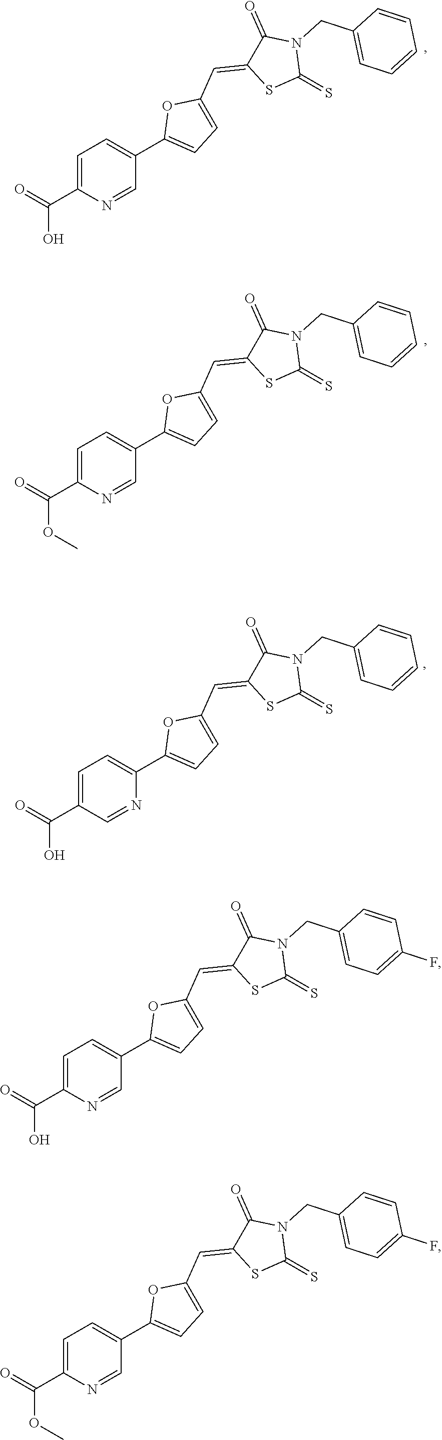 [SMILES: O=C(O)c1ccc(-c2ccc(/C=C3\SC(=S)N(Cc4ccccc4)C3=O)o2)cn1 ; COC(=O)c1ccc(-c2ccc(/C=C3\SC(=S)N(Cc4ccccc4)C3=O)o2)cn1 ; O=C(O)c1ccc(-c2ccc(/C=C3\SC(=S)N(Cc4ccccc4)C3=O)o2)nc1 ; O=C(O)c1ccc(-c2ccc(/C=C3\SC(=S)N(Cc4ccc(F)cc4)C3=O)o2)cn1 ; COC(=O)c1ccc(-c2ccc(/C=C3\SC(=S)N(Cc4ccc(F)cc4)C3=O)o2)cn1]
and pharmaceutically acceptable salts thereof.

40. The compound of claim 24, having a structure according to Formula XIId 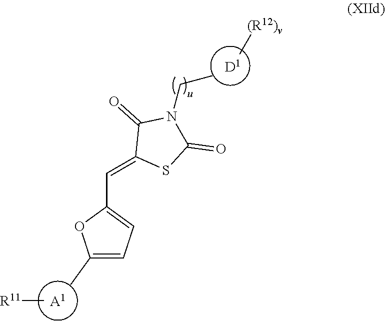
or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

41. The compound of claim 40, wherein subscript v is 0, 1, 2, or 3.

42. The compound of claim 40, wherein ring A¹ is selected from the group consisting of pyridin-2-yl and pyridin-3-yl.

43. A compound according to Formula XIII: 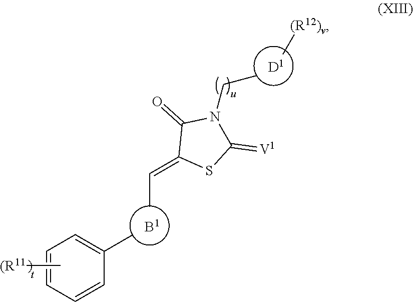
or a pharmaceutically acceptable salt thereof, wherein:
ring B¹ is selected from the group consisting of phenylene and C₅₋₆ heteroarylene, each of which is optionally substituted with one or two C₁₋₄ alkyl;
ring D¹ is selected from the group consisting of cyclohexyl and phenyl;
V¹ is selected from the group consisting of O and S;
each R″ is independently selected from the group consisting of halogen, (CH₂)ₛCOOR^{11a}, and C₅₋₆ heteroaryl, wherein C₅₋₆ heteroaryl is optionally substituted with (CH₂)ₛCOOR^{11a},
provided that at least one R¹¹ is other than halogen;
each R^{11a} is independently selected from the group consisting of H and C₁₋₆ alkyl;
each subscript s is 0, 1, or 2;
each R¹² is independently selected from the group consisting of halogen, C₁₋₆ alkyl, C₁₋₆ alkoxy, and R₁₂ₐ;
each R^{12a} is independently selected from the group consisting of C₂₋₆ alkyl and C₂₋₆ alkoxy;
subscript t is 0, 1, 2, 3, 4, or 5;
subscript u is 0, 1, or 2; and
subscript v is 0, 1, 2, 3, 4, or 5;
provided that when V¹ is O, ring B¹ is phen-1,5-diyl, R¹¹ is 4-carboxy, and subscript t is 1,
(R¹²)ᵥ-(ring D¹)-(CH2)ᵤ— is other than 4-methoxybenzyl;
provided that when V¹ is S, ring B¹ is thiophen-2,5-diyl, R¹¹ is 4-(COOMe), and subscript t is 1,
(R¹²)ᵥ-(ring D¹)-(CH₂)ᵤ— is other than 3,4,5-trimethoxybenzyl;
provided that when V¹ is S, ring Bⁱ is furan-2,5-diyl, R¹¹ is 4-carboxy, and subscript t is 1,
(R¹²)ᵥ-(ring D¹)-(CH₂)ᵤ— is selected from the group consisting of unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{12a})ᵥ-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOMe), and subscript t is 1,
(R¹²)ᵥ-(ring D¹)-(CH₂)ᵤ— is selected from the group consisting of 4-chlorophenyl, 4-fluorophenyl, 4-methylphenyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 3-methylbenzyl, 2-methylbenzyl, 2,4-dimethylbenzyl, 3,4-dimethylbenzyl, 2,3-dimethylbenzyl, (R^{12a})_{z}-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOEt), and subscript t is 1,
(R¹²)ᵥ-(ring D¹)-(CH₂)ᵤ— is selected from the group consisting of unsubstituted cyclohexymethyl, 4-chlorobenzyl, 3-chlorobenzyl, 3,4-dichlorobenzyl, 4-isopropylbenzyl, 3-methoxybenzyl, 2-methoxybenzyl, 2-methylbenzyl, 2,4-dimethylbenzyl, 3,4-dimethylbenzyl, 2,3-dimethylbenzyl, (R^{12a})ᵥ-phenyl, (R^{12a})ᵥ-benzyl, and (R^{12a})ᵥ-cyclohexylmethyl, wherein subscript v is 1, 2, or 3;
provided that when V¹ is S, ring B¹ is furan-2,5-diyl, R¹¹ is 4-(COOnBu), and subscript t is 1,
(R¹²)ᵥ-(ring D¹)-(CH₂)ᵤ— is selected from the group consisting of unsubstituted phenethyl, unsubstituted cyclohexymethyl, 4-chlorobenzyl, 3-chlorobenzyl, 2-chlorobenzyl, 3,4-dichlorobenzyl, 4-fluorobenzyl, 4-isopropylbenzyl, 4-methoxybenzyl, 3-methoxybenzyl, 2-methoxybenzyl, 4-methylbenzyl, 3-methylbenzyl, 2-methylbenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-carboxy, and subscript t is 1,
(R¹²)ᵥ-(ring D¹)-(CH₂)ᵤ— is selected from the group consisting of 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-(COOMe), and subscript t is 1,
(R¹²)ᵥ-(ring D¹)-(CH₂)ᵤ— is selected from the group consisting of 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, is 4-(COOEt), and subscript t is 1,
(R¹²)ᵥ-(ring D¹)-(CH₂)ᵤ— is selected from the group consisting of 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3;
provided that when V¹ is O, ring B¹ is furan-2,5-diyl, R¹¹ is 4-(COOnBu), and subscript t is 1,
(R¹²)ᵥ-(ring D¹)-(CH₂)ᵤ— is selected from the group consisting of 4-chlorophenyl, 4-fluorophenyl, 4-methoxyphenyl, 4-methylphenyl, unsubstituted phenethyl, unsubstituted cyclohexymethyl, 4-isopropylbenzyl, 4-methoxybenzyl, 3-methoxybenzyl, 2-methoxybenzyl, 2,4-dimethylbenzyl, 3,4-dimethylbenzyl, 2,3-dimethylbenzyl, (R^{12a})ᵤ-phenyl, (R^{12a})ᵤ-benzyl, and (R^{12a})ᵤ-cyclohexylmethyl, wherein subscript u is 1, 2, or 3.

44. The compound of claim 43, having a structure according to Formula XIVa 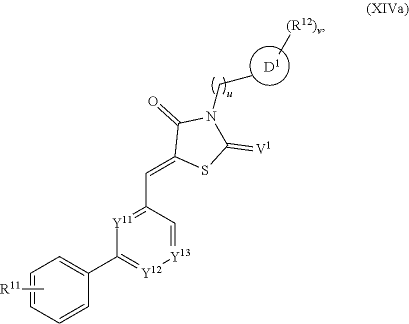
or a pharmaceutically acceptable salt thereof, wherein
R¹¹ is COOR^{11a};
R^{11a} is selected from the group consisting of H and C₁₋₆ alkyl;
each R¹² is independently selected from the group consisting of halogen, C₁₋₆ alkyl, and C₁₋₆ alkoxy; and
Y¹¹, Y¹², and Y¹³ are independently selected from the group consisting of CH and N.

45. The compound of claim 44, wherein subscript v is 0, 1, 2, or 3.

46. The compound of claim 44, wherein Y¹¹, Y¹², and Y¹³ are CH.

47. The compound of claim 44, wherein Y¹¹ is N, and wherein Y¹² and Y¹³ are CH.

48. The compound of claim 44, wherein Y¹¹ and Y¹² are N, and wherein Y¹³ is CH.

49. The compound of claim 44, wherein R^{11a} is selected from the group consisting of COOH and COOMe.

50. The compound of claim 43, selected from the group consisting of 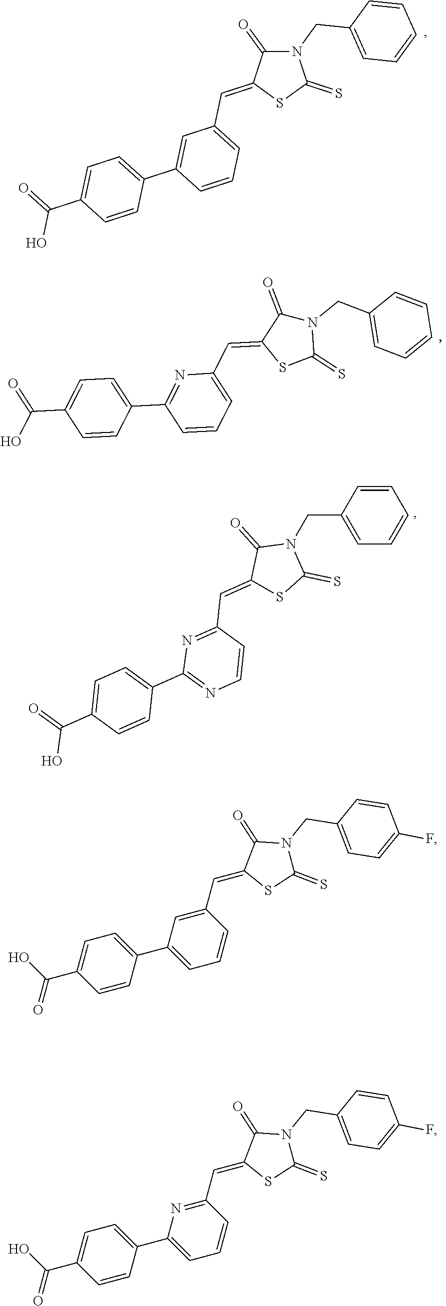 [SMILES: O=C(O)c1ccc(-c2cccc(/C=C3\SC(=S)N(Cc4ccccc4)C3=O)c2)cc1 ; O=C(O)c1ccc(-c2cccc(/C=C3\SC(=S)N(Cc4ccccc4)C3=O)n2)cc1 ; O=C(O)c1ccc(-c2nccc(/C=C3\SC(=S)N(Cc4ccccc4)C3=O)n2)cc1 ; O=C(O)c1ccc(-c2cccc(/C=C3\SC(=S)N(Cc4ccc(F)cc4)C3=O)c2)cc1 ; O=C(O)c1ccc(-c2cccc(/C=C3\SC(=S)N(Cc4ccc(F)cc4)C3=O)n2)cc1] 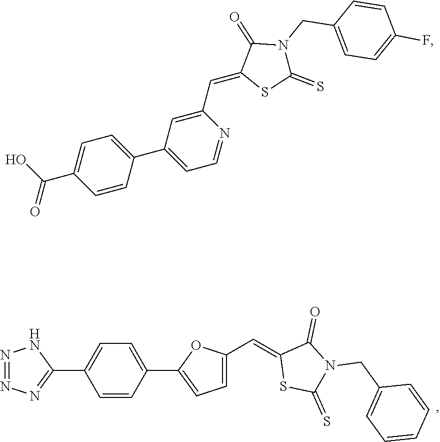 [SMILES: O=C(O)c1ccc(-c2ccnc(/C=C3\SC(=S)N(Cc4ccc(F)cc4)C3=O)c2)cc1 ; O=C1/C(=C/c2ccc(-c3ccc(-c4nnn[nH]4)cc3)o2)SC(=S)N1Cc1ccccc1]
and pharmaceutically acceptable salts thereof.

51. The compound of claim 43, selected from the group consisting of 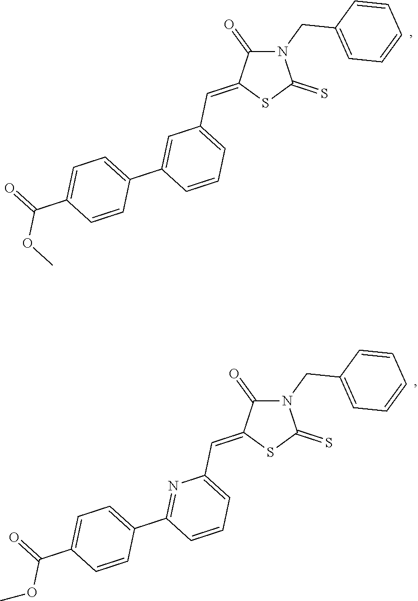 [SMILES: COC(=O)c1ccc(-c2cccc(/C=C3\SC(=S)N(Cc4ccccc4)C3=O)c2)cc1 ; COC(=O)c1ccc(-c2cccc(/C=C3\SC(=S)N(Cc4ccccc4)C3=O)n2)cc1]
and pharmaceutically acceptable salts thereof.

52. The compound of claim 43, having a structure according to Formula XIVb 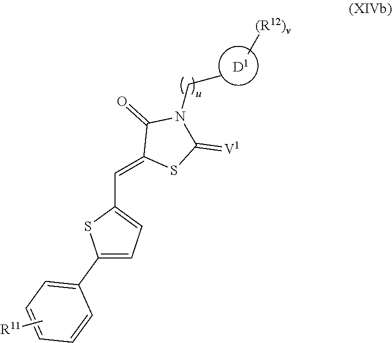
or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

53. The compound of claim 52, wherein subscript v is 0, 1, 2, or 3.

54. The compound of claim 52, which is 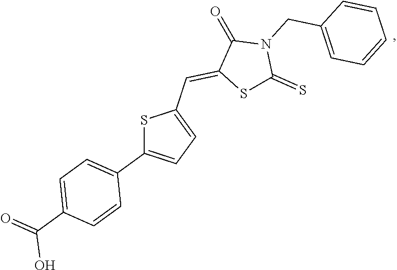 [SMILES: O=C(O)c1ccc(-c2ccc(/C=C3\SC(=S)N(Cc4ccccc4)C3=O)s2)cc1]
or a pharmaceutically acceptable salt thereof.

55. The compound of claim 43, having a structure according to Formula XIVc 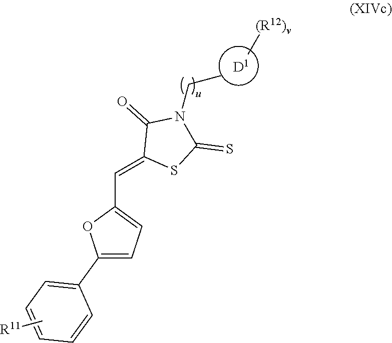
or a pharmaceutically acceptable salt thereof, wherein
R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

56. The compound of claim 55, wherein subscript v is 0, 1, 2, or 3.

57. The compound of claim 55, wherein R^{11a} is selected from the group consisting of COOH and COOMe.

58. The compound of claim 55, selected from the group consisting of: 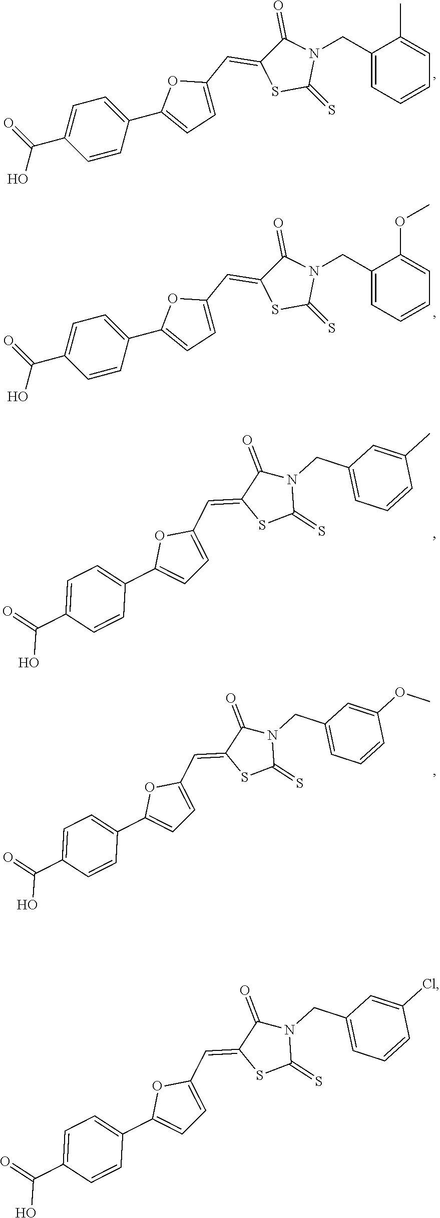 [SMILES: Cc1ccccc1CN1C(=O)/C(=C/c2ccc(-c3ccc(C(=O)O)cc3)o2)SC1=S ; COc1ccccc1CN1C(=O)/C(=C/c2ccc(-c3ccc(C(=O)O)cc3)o2)SC1=S ; Cc1cccc(CN2C(=O)/C(=C/c3ccc(-c4ccc(C(=O)O)cc4)o3)SC2=S)c1 ; COc1cccc(CN2C(=O)/C(=C/c3ccc(-c4ccc(C(=O)O)cc4)o3)SC2=S)c1 ; O=C(O)c1ccc(-c2ccc(/C=C3\SC(=S)N(Cc4cccc(Cl)c4)C3=O)o2)cc1] 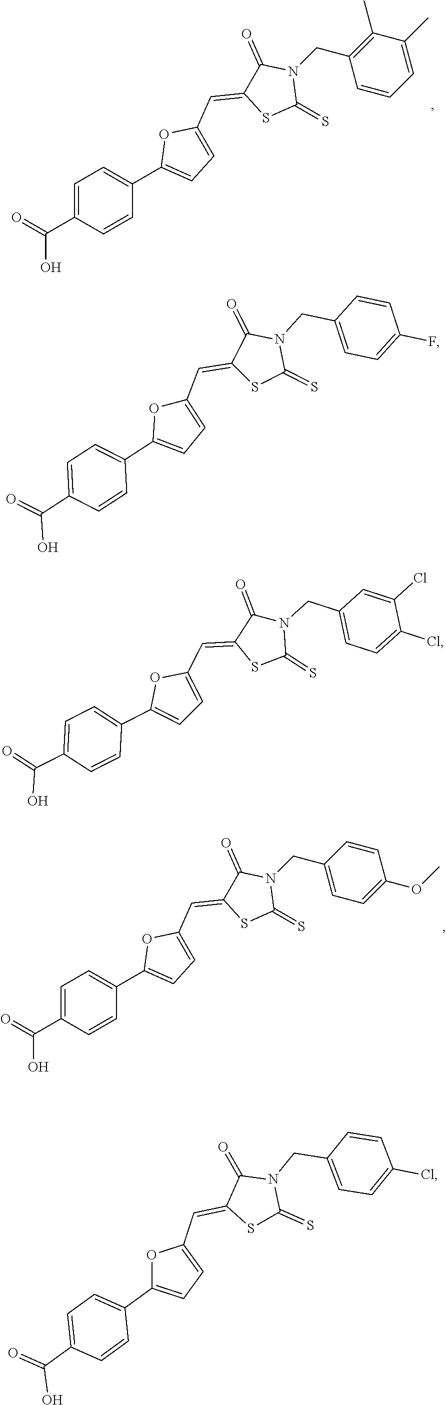 [SMILES: Cc1cccc(CN2C(=O)/C(=C/c3ccc(-c4ccc(C(=O)O)cc4)o3)SC2=S)c1C ; O=C(O)c1ccc(-c2ccc(/C=C3\SC(=S)N(Cc4ccc(F)cc4)C3=O)o2)cc1 ; O=C(O)c1ccc(-c2ccc(/C=C3\SC(=S)N(Cc4ccc(Cl)c(Cl)c4)C3=O)o2)cc1 ; COc1ccc(CN2C(=O)/C(=C/c3ccc(-c4ccc(C(=O)O)cc4)o3)SC2=S)cc1 ; O=C(O)c1ccc(-c2ccc(/C=C3\SC(=S)N(Cc4ccc(Cl)cc4)C3=O)o2)cc1] 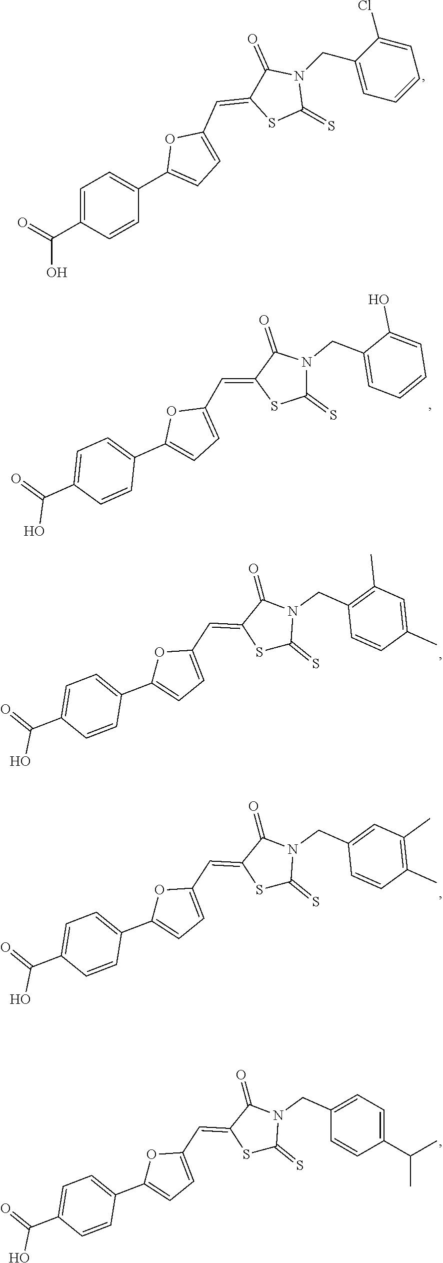 [SMILES: O=C(O)c1ccc(-c2ccc(/C=C3\SC(=S)N(Cc4ccccc4Cl)C3=O)o2)cc1 ; O=C(O)c1ccc(-c2ccc(/C=C3\SC(=S)N(Cc4ccccc4O)C3=O)o2)cc1 ; Cc1ccc(CN2C(=O)/C(=C/c3ccc(-c4ccc(C(=O)O)cc4)o3)SC2=S)c(C)c1 ; Cc1ccc(CN2C(=O)/C(=C/c3ccc(-c4ccc(C(=O)O)cc4)o3)SC2=S)cc1C ; CC(C)c1ccc(CN2C(=O)/C(=C/c3ccc(-c4ccc(C(=O)O)cc4)o3)SC2=S)cc1]
and pharmaceutically acceptable salts thereof.

59. The compound of claim 55, having the structure 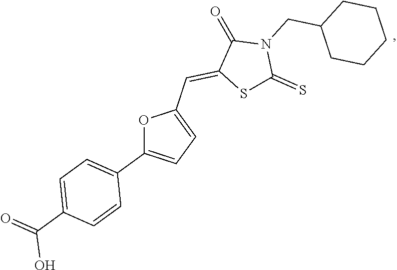 [SMILES: O=C(O)c1ccc(-c2ccc(/C=C3\SC(=S)N(CC4CCCCC4)C3=O)o2)cc1]
and pharmaceutically acceptable salts thereof.

60. The compound of claim 43, having a structure according to Formula XIVd 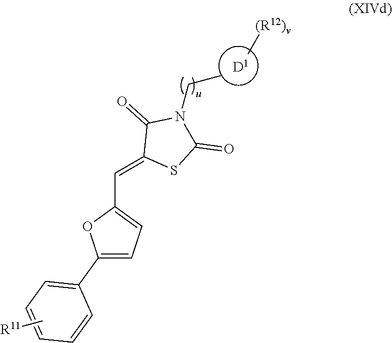
or a pharmaceutically acceptable salt thereof, wherein R¹¹ is COOR^{11a};
R^{11a} is selected from the group consisting of H and C₁₋₆ alkyl; and
each R¹² is independently selected from the group consisting of halogen, C₁₋₆ alkyl, and C₁₋₆ alkoxy.

61. The compound of claim 60, wherein subscript v is 0, 1, 2, or 3.

62. A pharmaceutical formulation comprising a pharmaceutically acceptable excipient and a compound according to any one of claims 1, 24, and 43 or a pharmaceutically acceptable salt thereof.

63. A method for treating a β2 integrin-mediated condition comprising administering to a patient in need thereof a compound according to any one of claims 1, 24, and 43 or a pharmaceutically acceptable salt thereof.

64. The method of claim 63, wherein the integrin-mediated condition is selected from the group consisting of acute inflammation, chronic inflammation, chronic kidney disease, neointimal thickening associated with vascular injury, atherosclerosis, tissue injury, peritonitis, diabetic nephropathy, an autoimmune disease, a neurological condition, lupus, cancer, pain, glaucoma, graft versus host disease, macular degeneration, and uveitis.
